SEQUENCE LISTING (163 sequences)

SEQ ID NO: 1 - protein, 374 aa, Bos taurus
MSPVRPRARAVLSLLGGLGPQLLLLLLRPLAASGDCSLPPDVPNAQAALGGLTSFPEKRTVTYKCNRGFVKVPGKADSVICENNKWSELAVFCNRSCDVPTRLLFASLKKSYSQQNYFPEGTTVEYECRQGYIRDPSLSGNITCLQNFVWSKPDRFCKKKSCPKPREIENGHVNITTDILLGATIYFSCNAGYKLVGATFSFCAPVGNTVGWTNEFPECQEILCPEPPKVDHGMIQEKQNSYAYGQSVTYKCMEGFTLHGESSIYCTVKDDQGEWSGPLPQCRETDVTPTVPKSTEVNVPAAKSTAVPRTTPRFHATGTPQGRGSAPSVASTVASGKCDSLRVHRICHRIRIYNSWKRILFFYCLCCCCSVTKS

SEQ ID NO: 2 - protein, 121 aa, Bos taurus
MASKGGYIVLGLLFLAVLCQLGHSLQCYSCINQVNCTSIVNCSHNQDACLFVKAVPLKFYHQCWKYEECSFEFIAKALGEKELHYDCCQKNLCNKSGGRSVSEKILLLLTLLLEAVCHFYR

SEQ ID NO: 3 - protein, 574 aa, Bos taurus
MNPGAARTPALRILALGALLWPAARPWELTILHTNDVHSRLEQTSEDSSKCVNASRCVGGVARLATKVHQIRRAEPHVLLLDAGDQYQGTIWFTVYKGTEVAHFMNALGYDAMALGNHEFDNGVEGLIDPLLKEVNFPILSANIKAKGPLASKISGLYSPYKILTVGDEVVGIVGYTSKETPFLSNPGTNLVFEDEITALQPEVDKLKTLNVNKIIALGHSGFEVDKLIAQKVKGVDVVVGGHSNTFLYTGNPPSKEVPAGQYPFIVTSDDGRKVPVVQAYAFGKYLGYLKVEFDEKGNVVTSHGNPILLNSSIPEDPNIKADINKWRVKLDNYSTQELGKTIVYLDGTAQSCRFRECNMGNLICDAMINNNLRHPDEMSWNHVSMCILNGGGIRSPIDERNNGTITWENLAAVLPFGGTFDLVQLKGSTLKKAFEHSVHRYGQATGEFLQVGGIHVVYDISRNPGDRVVKLEVLCTQCRVPSYEPLRMDKVYKVILPSFLVSGGDGFQMIKDEKIKHDSGDQDINVVSGYISKMKVLYPAVEGRIQFSAGSHCCGSFSLIFLSVLAVIIILYQ

SEQ ID NO: 4 - protein, 553 aa, Bos taurus
MRMLRRHHISFRSFAGSSGTPQAVFTFLLLPCCLALDFRAPPLISNTSFLWAWNAPVERCVNRRFQLPPDLRLFSVKGSPQKSATGQFITLFYADRLGYYPHIDEKTGKTVFGGIPQLGNLKSHLEKAKNDIAYYIPNDSVGLAVIDWENWRPTWARNWKPKDVYRDESVELVLQKNPQLSFPEASKIAKVDFETAGKSFMQETLKLGKLLRPNHLWGYYLFPDCYNHNHNQPTYNGNCPDVEKRRNDDLEWLWKESTALFPSVYLNIRLKSTQNAALYVRNRVQEAIRLSKIASVESPLPVFVYARPVFTDGSSTYLSQGDLVNSVGEIVSLGASGIIMWGSLNLSLSMQSCMNLGTYLNTTLNPYIINVTLAAKMCSQVLCHNEGVCTRKHWNSSDYLHLNPMNFAIQTGEGGKYTVPGTVTLEDLQKFSDTFYCSCYANIHCKKRVDIKNVHSVNVCMAEDICIDSPVKLQPSDHSSSQEASTTTFSSISPSTTTATVSPCTPEKHSPECLKVRCSEVIPNVTQKACQSVKLKNISYQSPIQNIKNQTTY

SEQ ID NO: 5 - protein, 295 aa, Bos taurus
MPLPLACLLLLLLSTHSALALKICSFNVRSFGESKKANCNAMDVIVKVIKRCDIILLMEIKDSSNRICPTLMEKLNGNSRKGITYNYVISSRLGRNTYKEKYAFLYKEKLVSVKQSYLYHDYQAGDADVFSREPFVVWFQSPYTAVKDFVIVPLHTTPETSVREIDELADVYTDVKRRWNAENFIFMGDFNAGCSYVPKKAWKDIRLRTDPKFVWLIGDQEDTTVKNSTNCAYDRIVLRGQNIVNSVVPQSNLVFDFQKAYRLSESKALDVSDHFPVEFKLQSSRAFTNSKKKKK

SEQ ID NO: 6 - protein, 527 aa, Bos taurus
MADNRDPASDQMKHWKEQRAAQKPDVLTTGGGNPVGDKLNSLTVGPRGPLLVQDVVFTDEMAHFDRERIPERVVHAKGAGAFGYFEVTHDITRYSKAKVFEHIGKRTPIAVRFSTVAGESGSADTVRDPRGFAVKFYTEDGNWDLVGNNTPIFFIRDALLFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLAHEDPDYGLRDLFNAIATGNYPSWTLYIQVMTFSEAEIFPFNPFDLTKVWPHGDYPLIPVGKLVLNRNPVNYFAEVEQLAFDPSNMPPGIEPSPDKMLQGRLFAYPDTHRHRLGPNYLQIPVNCPYRARVANYQRDGPMCMMDNQGGAPNYYPNSFSAPEHQPSALEHRTHFSGDVQRFNSANDDNVTQVRTFYLKVLNEEQRKRLCENIAGHLKDAQLFIQKKAVKNFSDVHPEYGSRIQALLDKYNEEKPKNAVHTYVQHGSHLSAREKANL

SEQ ID NO: 7 - protein, 403 aa, Bos taurus
MALQTAPPRLGSWGTPAHGSLLLLLLCLGWVLPSRAQAADVGLALLSSSVQRTSRDLSWQQPELTVLLPRARRGTEKKACPPGREAQVVDENLFFYEEWELEACVDGALLATQMRQVNLIPFTYQQLHIFKRKLDEFYPQGYPQSLIERLSYFFLYVTPEDIHKWNVTSLDTVKSLLKVSQGRGVDAQVAALIARYVGGGGQLDKAALDTLAIFRPAYLCFLRPEQLDSVQLSVLWMTTPQDLDACSPPQMAVLYHKAHMAFQNVSGSEYFTRIKPFLGGASTEDLRVFTSQNISIDAAAFKKLTTEAVLSLTVAEVQKLLGPNLVGLKAETGNMFLRDWISRQSQEDLDRLGLGLVGGVPNGYLVLDLRGREASSGGPHRLGQGPGPVLTVTASLLLVLVLS

SEQ ID NO: 8 - protein, 652 aa, Bos taurus
MLRGSAGAWAVLGPLLWGCGLSLLQGGMLYPRESRSRERKELDGLWSFRADFSDNRRQGFEQQWYRAPLRESGPTLDMPVPSSFNDVGQDGQLRSFVGWVWYEREITLPQRWTEDLGTRVVLRIGSAHYYAIVWVNGVHVLEHEGGHLPFEADISKLVQSGPLSSCRITIAINNTLSPHTLPPGTILYKTDPSMYPKGYFVQNTKFDFFNYAGLHRSVLLYTTPTTYIDDITVTTDMDQDIGLVNYQIIVQGSDHFQVDVSLLDEEGKVMAKGAGAEGQLQVPSAHLWWPYLMHEHPAYLYSLEVKLTAQTAVGPVSDFYTLPVGIRTVAVTKSQFLINGKPFYFRGVNKHEDADIRGKGFDWPLLVKDFNLLRWLGANAFRTSHYPYSEEVLQLCDRYGIVVIDESPGVGIVLVESFSNVSLQHHLEVMEEMIRRDKNHPAVVMWSLANEPASFLKPAGYYFKTLIAHTKALDPSRPVTFVTNTNYEADLGAPYVDIICVNSYYSWYHDYGHMEVIQLQLATQFENWYKAYQKPMIQSEYGADAIEGFHEDPPLMFSEEYQKGLLQQYHVVLDQKRKEYVVGELIWNFADFMTNQSPVRMIGNRKGIFTRQRQPKSAAFLLRERYWKLANETRYQRSAVTSQCVGSGLFTV

SEQ ID NO: 9 - protein, 342 aa, Bos taurus
KATMRKRTLKVLTLLLLFIFLTSFFLNYSHTMLATTWFPKQMVHELSENFKKLMKYSNRPCTCTRCIGQRRVSSWFDERFNRSMQPLLTAKNALLEEDTYNWWLRLQREKQPNNLNDTIKELFQVVPGDVDPLLEKGSVGCRRCAVVGNSGNLRESWYGPQIDSHDFVLRMNKAPTAGFEADVGRKTTHHLVYPESFRELAENVSMVLVPFKTVDLEWVISATTKGTISHTYVPVPAKIKVKKNKILIYHPAFIKYVFDSWLQGHGRYPSTGILSVIFSLHVCDEVDLYGFGADSKGNWHHYWENNPSAGAFRKTGVHDGDFESNVTATLASINKIRIFKGR

SEQ ID NO: 10 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 11 - protein, 555 aa, Bos taurus
MAPLRLFWIWLLVVGTRGVKDGDMRLADGGSANQGRVEIYYNGQWGTVCENMWDLTDASVVCRALGFQNATEALGGAAFGPGYGPIMLDEVRCTGTEPSLANCSSLGWMRSNCRHDKDASVICTNETRGVYTLDLSGELPAALEQIFESQKGCDLFITVKVREEDEIAMCAHKLILSTNPEAHGLWKEPGSRVTMEVDAECVPVVKDFIRYLYSRRIDVSLSSVKCLHKLASAYQAKQLQSYCGHLFAILIPQDPSFWTPLELYAYALATRDPVLEEICVQFLAWNFGALTQAEAWPSVPPALLQGLLSRTELVVPSELVLLLAVDKWSQERRTSHKEVEALVGQVRFPMMPPQDLFSLQFNLSLYWSHEALFQKKILQALEFHTVPFELLAQYWGLNLTEGTYQPRLYTSPTWSQSVMSSSYNPSRSFQTPQHPSFLFHDSSVSWSFVYLPTLQSCWNYGFSCSSDDPPLLALSKSSYSKSNPTIGYENRALLHCEGSFVVDVIDFKGWKALVPSALATNSSRSTSLFPCPSGVFSRFQVVIRPFYLTNSTDMD

SEQ ID NO: 12 - protein, 726 aa, Bos taurus
MRLLLLLAPLGWLLLAETKGDAKPEDNLLVLTVATKETEGFRRFKRSAQFFNYKIQALGLGEDWPGEAMLAGGGLKVRLLKKALEKHADKENLVILFTDSYDVVFASGPRELLKKFRQARSQVVFSAEELIYPDRRLEAKYPVVSDGKRFLGSGGFIGYAPNLSKLVAEWEGQDSDSDQLFYTKIFLDPEKREQINITLDHRCRIFQNLDGALDEVVLKFEMGQVRARNLAYDTLPVLIHGNGPTKLQLNYLGNYIPRFWTFETGCAVCDEGLRSLKGIGDEALPAVLVGVFIEQPTPFLSLFFQRLLRLHYPQKRLRLFIHNHEQHHKAQVEQFLAEHGDEYQSVKLVGPEVRVANADARNMGADLCRQDRGCTYYFSVDADVALTEPKTLRLLIEQNKNVIAPLMTRHGRLWSNFWGALSADGYYARSEDYVDIVQGRRVGVWNVPYISNIYLIKGSALRAELQETDLFHHSKLDPDMAFCANIRQQDVFMFLTNRHSFGHLLSLDSYQTTHLHNDLWEVFSNPEDWKEKYIHENYTKALAGKMVEMPCPDVYWFPIFTETACDELVEEMEHYGQWSLGDNKDNRIQGGYENVPTIDIHMNQINFEREWHKFLVEYIAPMTEKLYPGYYTRAQFDLAFVVRYKPDEQPSLVPHHDASTFTINIALNRVGVDYEGGGCRFLRYNCSIRAPRKGWTLMHPGRLTHYHEGLPTTKGTRYIAVSFVDP

SEQ ID NO: 13 - protein, 474 aa, Bos taurus
MPPSGPRAALFLLPSLLLLRAVLAVPLERGAPKEENPATESPDTGLYYHRYLQEVINVLETDGHFREKLQAANAEDIKSGKLSRELDFVSHHVRTKLDELKRQEVSRLRMLLKAKMDAQQEPNIQLDHLNLLKQFEHLDPQNQHTFEARDLELLIQTATRDLAQYDAAHHEEFKRYEMLKEHERRRYLESLGEEQRKEAERKLEEQQRRHREHPKVNVPGSQAQLKEVWEELDGLDPNRFNPKTFFILHDINSDGVLDEQELEALFTKELEKVYDPKNEDDDMREMEEERLRMREHVMKNVDTNQDRLVTLEEFLASTQRKEFGDTGEGWEQGKAGVPLPMAPVLTLQTVEMHPAYTEEELRRFEEELAAREAELNAKAQRLSQETEALGRSQGRLEAQKRELQQAVLQMEQRKQQQQSHNNPAPGPEGQLKFHPDTDDVPVPAPAGDQKDVDASEKKVPEQTPEPPQLDSQHL

SEQ ID NO: 14 - protein, 205 aa, Bos taurus
MEFFWASLLGLCCSLAAANRHTVFWNSSNPKFWNEDYTVHVRIDDYLDIICPHYEDNSVPDAAMEQYTLYLVEHEQYQLCQPQPKDHARWFCKSPKAKHGPEKLSEKFHRFTGFTLSKDFKEGHSYYYISKPIHHQEDRCLRLKVMIAGKITHSPQAHPNAQEKRLPADDPEVQVLHSIGHSAAPRLFPLAWAVLLLPFLLLQIP

SEQ ID NO: 15 - protein, 109 aa, Bos taurus
DQDEGVSTEPTQDGPAELPEDEECVFPFVYRNRKHFDCTVHGSLFPWCSLDADYVGRWKYCAQRDYAKCVFPFIYGGKKYETCTKIGSMWMSWCSLSPNYDKDRAWKYC

SEQ ID NO: 16 - protein, 404 aa, Bos taurus
MRLCLFLCLVLLGPRMATLRRSQKKKIQEVPPAVTTAPPGSRDFVFDLYRALAAAAPAQNIFFSPLSITVSLAMLSLGAQSNTKAQILEGLGIGPGEGSEEELHSASQRLLRELQQPQDSLQLSLGNALFTKPRLPIQEAFLGAMRTLYLADTFPTNFEDPEGAKKKINDYVAKQTKGKIVDLIKSLDGTQVMVMVNYIFFKAKWETSFNLKSTHEQDFYVTPETVVRVPMMKQQDQFYYLLDRNLSCKVVGVPYQGNATAFFILPREGEMEQVENGLKEKTLKKWLRMPMKRRLELYLPKFSIEGSYQLEEVLPKLGIRDIFTSDADLTGISNHSSIRVSEMVHKAVVEVDESGTQAAAATGMVITFKSARLGSQRIVFNRPFLVLIVKNSKHILFLGKVTRP

SEQ ID NO: 17 - protein, 241 aa, Bos taurus
MILRKKLSYLWTLGLCLVASKSPPKAPSITNDRFIEECLRLHNEARTNVSPPAADMKYMSWDEALAKTAEAWAKKCKFIHNSCSSKSFKCHPTFQYAGENLWLGPLTISAAKFAINMWYDERKFYDFNTRSCSQVCGHYTQVVWAYSYKVGCAVAVCPNLGSPDSALLVCNYAPAGNYPNMSPYTNGTPCSMCQGDTCENNLCRNKERDKSQRYPNWNPSGTRQLIACNPLYLISVLLTIF

SEQ ID NO: 18 - protein, 278 aa, Bos taurus
MRIAVICFCLLGIASALPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKAKDKNKHSNLIESQENSKLSQEFHSLEDKLDLDHKSEEDKHLKIRISHELDSASSEVN

SEQ ID NO: 19 - protein, 440 aa, Bos taurus
KMKTLLLLMGLLLSWESGWAISDKELQEMSTEGSKYVNKEIKNALKEVKQIKTQIEQTNEERKLLLSSLEEAKKKKEDALNDTRDSENKLKASQGVCNETMTALWEECKPCLKQTCMKFYARVCRSGSGLVGHQLEEFLNQSSPFYFWINGDRIDSLMENDREQSHVMDVMEDSFTRASSIMDELFQDRFFLRRPQDTQYYSPFSSFPRGSLFFNPKSRFARNVMPFPLLEPFNFHDVFQPFYDMIHQAQQAMDAHLQRTPYHFPTMEFTENNDRTVCKEIRHNSTGCLRMKDQCEKCQEILEVDCSASNPTQTLLRQQLNASLQLAEKFSRLYDQLLQSYQQKMLNTSALLKQLNEQFTWVSQLANLTQSDDQHYLQVFTVNSHNSDPSIPSGLTKVIVKLFNSFPITVTVPQEVSSPNFMENVAEKALQQYRRKSQEE

SEQ ID NO: 20 - protein, 998 aa, Bos taurus
MGSVLTQLLLLWLLGTQPEATIRVFVVPHSHLDVGWLHTVQGSTQAYVTTVYTSVVEDLTRNKQHRFIIVDQEFFRLWWDGVASAKQKVQVRQLVDQGRLEFVLGGQVMHDEAVTHVDDQILQLTEGHRFLYETFGIRPQFSWQVDSFGASATTPTLFALAGFNGHIISRIDYDLKETMQQDQEMQFVWRGSRSLLAQQEMFTHVLDENGYCGVSSNISLLLSKGFYWNEKAVFPDPPWDGMYPNQTVPVTLSNIQRYANTLSYNVRKRAPWFLTPHILWPWGCDRQFFNASVQFANMDYLMGYINKHVSQFGVLMVYATLSEYFQTLHSEHEPVQVRDHRDFLPYSSDMFHSWTGFYASRSGLKGLARRASALLYAGESMFTRFMLAPHRFLDRAWGLQQLQKLRWAVSEVQHHEAITGAHTPKVSDMFVGRLNNGMHGVQKLMSAIVQDRFPAHTGLEPGGYVAVIYNPLVWTVTTIVTLTVDFPKVSVTDESGHPVPAQVQNSKEMPSVYDLLVLTTIPGLSYRYYVIKPSRRAKGDTQVTEATMANTKQFGRRPRRHDSSVGRRLVHVENDCYSVFLDKDTNLMHSIWERQSNQTIQVTQQFMEYEANGDEDQGPISDNYVFTSSETAKPAWETVGMEIVEGELVTEIRQYFYRTVNDRDPTYTVYSRLARGPPGADGELLCHRIEQEYRVGPLELNREAIWRTSTNLNTARVLYSDNNGYQVQRRAYKHHENNIARNYYPMTQSAFIQDRQSRLVLLSEQAHGVSSQGSGQMEVMLHRRLLIKQQWALSVNVTLNDTSVVHSVLWLLLGPSTLTRDLGQRSGVALQHRPVVLIRELSETARIHPHPQHQEAVTLPPSIHLQILSIPGWTYNSNHTKHVKNLRKDNKGEAKAEFRRVLLRLHHLYEADEDPVRSQPVTVNLQSVLQGLGSVVSVEERSLTGTWDVSTLHRWRWPTQEPGYLRGNSSHPSLHPGSFTITIHPKEIRTFFIYFKER

SEQ ID NO: 21 - protein, 241 aa, Bos taurus
MSMLFYTLITALLIGIQAAPHTESNVPAGHAIPQAHWIKLQHSLDTVLRRAHSAPAGPIAARVAGQTHNITVDPKLFKKRRLRSPRVLFSTQPPPVAADTQDLDFEAGGASSFNRTHRSKRSSSHPVFHRGEFSVCDSISVWVGDKTTATDIKGKEVMVLGEVNINNSVFKQYFFETKCRDPNPVDSGCRGIDAKHWNSYCTTTHTFVKALTMDGKQAAWRFIRIDTACVCVLSRKTGQRA

SEQ ID NO: 22 - protein, 761 aa, Bos taurus
MGIGHNPRLCLVPLILGLLCGGVGMTPLPEAGPQSPCSLEGVEIKGGSFRLLKAGQVLEYLCPSGFYPYPTQIRTCRSTGSWSTLQTQDRKIVKRAECKAIRCPRPQDFENGEYWPRAAYYNLSDEISFRCYDGYTLRGSANRTCQGNGRWDGETAICDDGATYCPNPGIPLGTRKVGSQYRLEDRVTYYCNRGLTLRGSEQRTCLEGGSWSGTEPSCQDSFMYDTPAEVAEAFLSSLTETIEGVDAEDGHSPGEQQKRKIVLDPSGSMNIYLVLDGSDSVGAHNFTGAKNCLRDFIEKVASYGVKPKYGLVTYATEPKVLIRVSDPKSSEADWVTDQLNQINYADHKLKAGTNTKRALLEVYNMMSREVNQFKETWNRTRHVIIIMTDGLHNMGGDPVTVIHDIRYLLDIGRNRKNPREDYLDIYVFGVGPLVNQENINALASKKDKEKHVFKLQGMENLEDVFVQMLDESRTLGLCGMVWEHKDGTAYHKQPWQAKISVTRPSKGHESCMGAIVSEYFVLTAAHCFTVDDEKHSIKVSLGGQRKEWEVKEILFHPKYDLNAKKAKGIPEFYDYDVALVRLKEKLKYETTIRPICLPCTEGSIQALRLPRSTTCQQQMQELLPAKDIEALFVSESKKTLTRKAVYIKNGDKKASCERDALRAPGYEKVKDVSEVVTPRFLCTGGVDPYADPNTCKGDSGGPLIIHKRSRFIQVGVISWGVVDVCKRPQQVPGYARDFHINLYQVLPWLKEKLQNEDLGFL

SEQ ID NO: 23 - protein, 134 aa, Bos taurus
MKLSSVIPWALLLSTASSDSTDGLLVKDKHLCGDLYGEEYGVIFPYLGLHTECLWIIKMDPGYRILLEVRDVHLNCNKESLEIIEGPPESSNSRKICDTSHAEYTSCTNTMTVKYTRKPNHPAPDFFLIFRRVL

SEQ ID NO: 24 - protein, 956 aa, Bos taurus
MILEERSSWEGSKRYCIKTKHVAIICAVVVAVGLIVGLSVGLTRSCDSTEGMTQGTTQGTTQAPSHLPPVTSPQEDQGVCPASEDESGGWKDFRLPDFIKPVHYDLEVKPLMEQDTYTGSVDISINVSSSTRYLWLHLRETRITRLPVLRRPSGEQVQVRQCFEYKKQEYVVVEAEEELEPNTGEGPYHLILEFAGWLNGSLVGFYRTTYVEKGQTKSIAATDHEPTDARKSFPCFDEPNKKATYTISIVHSKEYKALSNMPVEKEESVDDTWSRTTFQKSVPMSTYLVCFAVHQFDSVTRISNRGIPLTIYVQPEQKHTAEYAANITKSVFDYFEDYFGMSYSLPKLDKIAIPDFGTGAMENWGLITYRETNLLYDPDESASSNKQRVAAVIAHELVHQWFGNIVTMEWWDDLWLNEGFASFFEYLGVAYAEKDWQMRDQMILDDVLPVQEDDSLMSSHPIVVTVATPDEITSVFDGISYSKGASILRMLENWITREKFQIGCQNYLKKHKFENAKTSDFWAALEEASNLPVKEVMDTWTNQMGYPVLNVDNMKNITQKRFLLDPRANASEPHSAFGYTWNIPIKWTEDDEQRITLYNRSETGGITLESTLSGNAFLKINPDHIGFYRVNYEVSTWEWIATNLSVNHTDFSSADRASFIDDAFALARAQLLNYKEALNLTKYLKEEKEYLPWHRVISAVTYIISMFEDDKELYPVIEKYFRDQVKPIADSLGWNDVGDHLTKLLRASVLGLACKMGDSDALNNASQLFQEWLTGTVSLPVNLRLLVYRYGMQNSGNETSWNYTLEQYQKTSLAQEKEKLLYGLASVKNVTLLSRYLDLLKDSNLIKTQDVFTVIQYISYNSYGKTMAWNWIQLNWEYLVNRYTLNNRNLGRIVTIAEPFNTELQLWQIKSFFERYPEAGAGQKPREQVLETVKNNIEWLKQNRDTIRNWFLDLNG

SEQ ID NO: 25 - protein, 399 aa, Bos taurus
MNWHFPFFLVASVTLPSVCSQFNPLSLEELGSDTGIQVFNQIVKSQPHDNIIISPHGIASVLGMLQLGADGRTKKQLTTVMRYGVNGVGKMLKKINKAIVSKKNKDIVMVANAVFVKNDSKMEVPFVTRNKDVFQCEVRNANFEDPASACASINAWVRNETQGMIDSLLSPDLLNGVLTRLVLVNAVYFKGLWRSRFLPENTKKRTFVAADGKSYQVPMLAQLSVFRCGSTSTPSGLWYNFIELPYHGESISMLIALPTESSVPLSAIVPHISTKTIDSWVSTMVPKRVQVILPKFTAVAQTDLKEPLKVLGITDMFDPSKANFAKITKTGSENLHVSHILQKAKIEVSEDGTKASAATTAILIARSSPPWFIVDRPFLFFIRHNPTGAVLFMGQINKP

SEQ ID NO: 26 - protein, 221 aa, Bos taurus
MAGCACGFVRAVLAFLLAPALVTLLVAPARGRGGRGHGDWDAAELLLPPLPPRENAALVARFVTNVCDWGALATISTQEGVRGRPFADVLSLSDGPPGKGSGVPYFYLSPLQQSVGNLQENPYATLTMSLAETNFCRKYGFDPQSPLCAHIILSGTVIKVSETEMDVAKNSLFIRHPEMKTWPSSHNWFFAKLNITNIWVLDYFGGPKIVTPAEYYNATSQ

SEQ ID NO: 27 - protein, 405 aa, Bos taurus
MTRTSLKKKVFSCCVLIFLLFAIICVWKEKKKGNYYEFLKLQNKEYQVLQGLEKLAVSSSSQPVSSSSTHNPQRNIQALGGPKAKLKATFQVWDKDSSSKNLAPRLQTIRKNYLNMNKYKVTYKGPGPGVKFSAEALLCHLRDHVNISMIEATDFPFNTSDWEGYLPQEDIRTKAGPWGRCAVVSSAGSLKSSRLGREIDDHDAVLRFNGAPTVKFQQDVGTKTTIRLVNSQLVTTEAGFLKDSLYNEGILIVWDPSVYHSDIPKWYRNPDYSFFNNFKSYRKLHPDQPFYILKPQMPWELWDIIQEISSELIQPNPPSSGMLGIAIMMSLCDQVDIYEFLPSKRKTDVCYYYQRYFDSACTMGAYHPLLFEKNMVKYLNLGTDEDIYLLGKATLPGFRTIRCGA

SEQ ID NO: 28 - protein, 99 aa, Bos taurus
MKVSAALLCLLLTVAAFSTEVLAQPDAINSQVACCYTFNSKKISMQRLMNYRRVTSSKCPKEAVIFKTILGKELCADPKQKWVQDSINYLNKKNQTPKP

SEQ ID NO: 29 - protein, 134 aa, Bos taurus
MKLSSVIPWALLLSTATVDSMDWLPRNTNCGGILKEESGVIATYYGPKTNCVWTIQMPPEYHVRVSIQYLQLNCNKESLEIIDGLPGSPVLGKICEGSLMDYRSSGSIMTVKYIREPEHPASFYEVLYFQDPQG

SEQ ID NO: 30 - protein, 220 aa, Bos taurus
MGAAARSLPLAFCLLLLGTLLPRADACSCSPVHPQQAFCNADIVIRAKAVNKKEVDSGNDIYGNPIKRIQYEIKQIKMFKGPDQDIEFIYTAPSSAVCGVSLDIGGKKEYLIAGKAEGNGNMHITLCDFIVPWDTLSATQKKSLNHRYQMGCECKITRCPMIPCYISSPDECLWMDWVTEKNINGHQAKFFACIKRSDGSCAWYRGAAPPKQEFLDIEDP

SEQ ID NO: 31 - protein, 422 aa, Bos taurus
GRGLLGAQVVPRAASSGVLCSLLVAGRMLGWSRLTFILLSGIVTCLVAQQVPPWTEDCRKSTYPPSGPTYRGPVPWYTINLDLPPYKRWHELMVVKAPALKVIVNSMKNIVNAFVPSGKIIHLVDQKLPGLLGNFPGPFEEEMKGIAAVTEIPLGEIILFNIFYEFFTICTSIITEDKEGHLLHGRNLDFGVFLGWNINNDTWVITEELKPLTVNLDFQRNNKTLFKATTFAGYVGMLTGFKPGLFSVTLNERFSIDGGFMGVMEWILGKKDAQWVGFIIRSVLENSTSYEETKNILTKTKILAPAYFILGGNQSGEGCVITRDRKQSLDIYELDPKHGRWYVVQTNYDRWKNPFFLDDRRTPAKMCLNQTTQENISFATIYDVLSTKPILNKLTVYTVLIDVTKGQFETYLRDCPDPCIGW

SEQ ID NO: 32 - protein, 596 aa, Bos taurus
PSSYSQSGSHSPALALSPPAAAMNPGAARTPALRILALGALLWPAARPWELTILHTNDVHSRLEQTSEDSSKCVNASRCVGGVARLATKVHQIRRAEPHVLLLDAGDQYQGTIWFTVYKGTEVAHFMNALGYDAMALGNHEFDNGVEGLIDPLLKEVNFPILSANIKAKGPLASKISGLYSPYKILTVGDEVVGIVGYTSKETPFLSNPGTNLVFEDEITALQPEVDKLKTLNVNKIIALGHSGFEVDKLIAQKVKGVDVVVGGHSNTFLYTGNPPSKEVPAGQYPFIVTSDDGRKVPVVQAYAFGKYLGYLKVEFDEKGNVVTSHGNPILLNSSIPEDPNIKADINKWRVKLDNYSTQELGKTIVYLDGTAQSCRFRECNMGNLICDAMINNNLRHPDEMSWNHVSMCILNGGGIRSPIDERNNGTITWENLAAVLPFGGTFDLVQLKGSTLKKAFEHSVYRYGQATGEFLQVGGIHVVYDISRNPGDRVVKLEVLCTQCRVPSYEPLRMDKEYKVILPSFLVSGGDGFQMIKDEKIKHDSGDQDINVVSGYISKMKVLYPAVEGRIQFSAGSHCCGSFSLIFLSVLAVIIILYQ

SEQ ID NO: 33 - protein, 299 aa, Bos taurus
MKTLQSTLLLFLFVPLIKPAPPSQQDSRIIYDYGTDNLEETFFSQDYEDKYLDGKSTKEKETMIIVPDEKSFQLQKDETITPLPPKKENDEMPTCLLCVCLSGSVYCEEVDIDAVPPLPKESAYLYARFNKIKKLTAKDFADIPNLRRLDFTGNLIEDIEDGTFSKLSLLEELTLAENQLLKLPVLPPKLTLFNAKYNKIKSRGIKANTFKKLHNLSFLYLDHNALESVPLNLPESLRVIHLQFNNITSITDDTFCKANDTSYIRDRIEEIRLEGNPVILGKHPNSFICLKRLPIGSYI

SEQ ID NO: 34 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 35 - protein, 471 aa, Bos taurus
MAGEMTILGSAVLTLLLAGYLAQQYLPLPTPKVIGIDLGTTYCSVGVFFPGTGKVKVIPDENGHISIPSMVSFTDDDVYVGYESLELADSNPQNTIYDAKRFIGKVFTPEELEAEIGRYPFKVLNKNGMVEFSVTSNETITVSPEYVGSRLLLKLKEMAEEYLGMPVANAVISVPAEFDLKQRNSTIQAANLAGLKILRVINEPTAAAMAYGLHKAEVFHVLVIDLGGGTLDVSLLNKQGGMFLTRAMSGNNKLGGQDFNQRLLQYLYKQIYQTYGFLPSRKEEIHRLRQAVEMVKLNLTLHETAQMSVLLTVEENDRKGPPTSDSELPKDKFSQANDPHVDSMFGANLSEKKNGEGQVLFETEISRKLFDTLNEDLFQKILVPIQQVLKEGHLEKTEIDEVVLVGGSTRIPRIRQVIQEFFGKDPNTSVDPDLAVVTGVAIQAGIDGGSWPLQVSALEIPNKHLQKTNFN

SEQ ID NO: 36 - protein, 244 aa, Bos taurus
MALFTVVLFLAAVWLPFFPAKGQDRRFADLSNTLKNVQTEIVNKHNDLRRGVSPPPSNMLKMQWNTTAAANAQNWANKCLFKHSKKEDRRVGTRNCGENLFMSSYPSTWSNAIQSWYDEVHDFVFEVGPKSPQAVIGHFTQIVWYSSFLIGCGVAYCPKQSLKYLYVCQYCPAGNIVGRQHVPYQKGTPCGSCPNHCDNGLCTNSCEYEDTYSNCASLKETWTCASDFVKTNCKAACNCQGKIY

SEQ ID NO: 37 - protein, 123 aa, Bos taurus
MPACRLGPLLAALLLGLLLGLPPVTGSIAVKPGECPELEGDANCTKACVLDEDCDDNLKCCQAGCATVCQMPNDKPGSCPNVDIAFPQLGLCRDQCQVDSQCPDALKCCVNGCGRVSCVTPVF

SEQ ID NO: 38 - protein, 149 aa, Bos taurus
MARPSLLLPMALALLALCLLALPRDARARPGDRKVGELQELSPNDPQVQKAAQVAVANYNMGSNSDYYYRDITILRAHSQLVAGIKYYLTVDMGSTACRKSAVAGDHVDLTTCPLAAEAQQEKLRCDFEILVVPWKNSSQLLKHDCVSL

SEQ ID NO: 39 - protein, 191 aa, Bos taurus
MATPNRLWMALLLLGVLGVLQTPAPAQAALQPNFEEDKFLGRWFTSGLASNSSWFLEKKKVLSMCKSVVAPAADGGLNLTSTFLRKDQCETRTLLLRPAGPPGCYSYTSPHWSSTHEVSVAETDYETYALLYTEGVRGPGQDFRMATLYSRSQNPRAEVKEHFTTFAKSLGFTEEGIVFLPKTDKCMEEHP

SEQ ID NO: 40 - protein, 510 aa, Bos taurus
MIMSQLLNYVAVLFFCVSRASSLDTFIAAVYEHAVILPNATLVPVSPEEALAVMNRNLDLLEGAVTSASKQGAHIIVTPEDGIYGFNFTRESIYPYLEDIPDPQVNWIPCNNPDRFGHTPVQQRLSCLAKDNSIYIVANIGDKKSCNASDPQCPPDGRYQYNTDVVFDTKGKLVARYHKQNLFLNEDQFNAPKEPEVVTFNTTFGKFGIFTCFDILFHDPAVTLVRDSRVDTILFPTAWMNVLPHLSAIEFHSAWAMGMRVNFLASNLHYPLKKMTGSGIYAPDSPRAFHYDMKTEEGKLLLAQLDSHPHPTPVVNWTSYASGVEAHSVGNQEFTGIIFFDEFTFLELKEIGGNYTVCQRDLCCHLSYKMSEKRSDEVYALGAFDGLHTVEGSYYLQICTLLKCKTTDLHTCGDSVETASTRFEMFSLSGTFGTQYVFPEVLLSEIQLAPGEFQVSNDGRLFSLKPTSGPVLTVTLFGRLYEKDSAPNTLSDLTTQALRLNPKTDAWKSK

SEQ ID NO: 41 - protein, 563 aa, Bos taurus
  UNSURE 563: Xaa is any amino acid
MGPRSGLLGLFALFVAGKCSYSPEPDQQRRLPPGWVSLGRADPEEELSLTFALRQQNVKRLSELVQAVSDPGSPRYGKYLTLEDVAELVRPSPLTLHTVQKWLLAAGARNCHSVTTQDFLTCWLSVRQAELLLSGAEFHHYVGGPAETHAVRSLHPYRLPKALAPHVDFVGGLHRFPPTSTLRQHPEPQVPGTVGLHLGVTPSVIRKRYNLTAQDVGSGTTNNSQACAQFLEQYFHDSDLAEFMRLFGGDFAHQASVARVVGQQGRGRAGIEASLDVEYLMSAGANISTWVYSSPGRHESQEPFLQWLLLLSNESALPYVHTVSYGDDEDSLSSTYIQRVNTELMKAAARGLTLLFASGDSGAGCWSVSGRHQFRPSFPASSPYVTTVGGTSFQNPFRVTDEVVDYISGGGFSNVFPRPSYQEEAVTRYLSSSPHLPPSSYFNASGRAYPDVAALSDGYWVVSNHVPIPWVSGTSASTPVFGGLLSLINEHRILRGLPPLGFLNPRLYQKHGAGLFDVTRGCHESCLNEEVEGQGFCSGPGWDPVTGWGTPNFPALLKTLMNX

SEQ ID NO: 42 - protein, 254 aa, Bos taurus
VPGSAVCKQIETMAILRSLLLPLGLLLCLWLLCSPASCTNSTTNCKPFLSVTTKKSSLIHISPDIYKSNTLYTVSTQVSHGIISVVLEARDQNKSVIGSWENSSDHCEGRAEYYLKGNFSTLFKAKWMSPNSTDITTVKINIYTVNSLRNAELDSITLPEVGTVTVKHVSTKQVITTPTHKPTPAPPKPTTNPQKTTTNHSIPTTSLPKPTTSLYTSHPKLTTTHKSSANRAFLCPVREAIQILFIFLIGTLLF

SEQ ID NO: 43 - protein, 234 aa, Bos taurus
MDSVRPLWLMLLSLLLVGTALGDASQAPPGNNAEICLLPPDDGPCRARIPSYYYDRYTQSCREFMYGGCEGNANNFETLEACNEACWKIEKVPKICRLKVNKKQCGELREQYFFNLSSMTCKKFISGGCHSNENRFPDEATCMDFCAPKRAPVFCYSPKDEGLCSANVTRYYFNPRHKACEAFNYTGCGGNDNNFVNLKDCKRTCVKALKKEKNKKMPRLLLANRRLKIKKKQF

SEQ ID NO: 44 - protein, 92 aa, Bos taurus
MKLVTVLMLVALPLYCYAGSSGCSLLDNVVEKSVDPTVSKDEYRAYLKDFAQTDVEKKAADELKQCFLQQSNETLANFKQMLQIIYDSIYCK

SEQ ID NO: 45 - protein, 938 aa, Bos taurus
PMMRWPPCSRPLLGVCTLLSLALLGHILLHDLEVVPRELRGFSQDEIHQACQPGASSPECRGSPRAAPTQCDLPPNSRFDCAPDKGITPQQCEARGCCYMPAEWPPDAQMGQPWCFFPPSYPSYRLENLTTTETGYTATLTRAVPTFFPKDIMTLRLDMLMETESRLHFTIKDPANRRYEVPLETPRVYSQAPFTLYSVEFSEEPFGVVVRRKLDGRVLLNTTVAPLFFADQFLQLSTSLPSQHITGLAEHLGSLMLSTNWTKITLWNRDIAPEPNVNLYGSHPFYLVLEDGGLAHGVFLLNSNAMDVVLQPSPALSWRSTGGILDVYIFLGPEPKSVVQQYLDVVGYPFMPPYWGLGFHLCRWGYSTSAITRQVVENMTRAYFPLDVQWNDLDYMDARRDFTFNKDHFGDFPAMVQELHQGGRRYIMIVDPAISSSGPAGTYRPYDEGLRRGVFITNETGQPLIGQVWPGLTAFPDFTNPETLDWWQDMVTEFHAQVPFDGMWIDMNEPSNFVRGSVDGCPDNSLENPPYLPGVVGGTLRAATICASSHQFLSTHYDLHNLYGLTEALASHRALVKARGMRPFVISRSTFAGHGRYSGHWTGDVWSNWEQLSYSVPEILLFNLLGVPLVGADICGFLGNTSEELCVRWTQLGAFYPFMRNHNALNSQPQEPYRFSETAQQAMRKAFTLRYVLLPYLYTLFHRAHVRGETVARPLFLEFPEDPSTWTVDRQLLWGEALLITPVLEAEKVEVTGYFPQGTWYDLQTVPMEAFGSLPPPAPLTSVIHSKGQWVTLSAPLDTINVHLRAGHIIPMQGPALTTTESRKQHMALAVALTASGEAQGELFWDDGESLGVLDGGDYTQLIFLAKNNTFVNKLVHVSSEGASLQLRNVTVLGVATAPQQVLCNSVPVSNFTFSPDTETLAIPVSLTMGEQFVISWS

SEQ ID NO: 46 - protein, 508 aa, Bos taurus
AMEALWTLTLALAAGLAAASPPNILLIFADDLGYGDLGSYGHPSSTTPNLDQLAAGGLRFTDFYVPVSLCTPSRAALLTGRLPVRMGLYPGVLEPSSRGGLPLDEVTLAEVLAAQGYLTGIAGKWHLGVGPEGAFLPPHHGFHRFLGIPYSHDQGPCQNLTCFPPATPCEGICDQGLVPIPLLANLSVEAQPPWLPGLEARYVAFARDLMTDAQHQGRPFFLYYASHHTHYPQFSGQSFPGHSGRGPFGDSLMELDAAVGALMTAVGDLGLLGETLVFFTADNGPETMRMSHGGCSGLLRCGKGTTFEGGVREPALAFWPGHIAPGVTHELASSLDLLPTLAALAGAQLPNITLDGVDLSPLLLGTGKSPRHTLFFYSAYPDEVRGVFAVRSGKYKAHFFTQGSVHSDTTADPACHASNPLTAHEPPLLFDLSEDPGENYNLLDSVDEVAPEALQAVKQLELLKAQFDAAMTFGPSQMAQGEDPTLQVCCQPSCTPRPSCCHCPEFQP

SEQ ID NO: 47 - protein, 519 aa, Bos taurus
MITSSLQTSVAVFALVILHVSTLDTFKAAVYEHAVILPNDTETPVSPDEALFLMNKNIDILEKAIKQAAEQQGAQIIVTPEDALYGWKFTRETIFPYLENIPDPQVNWIPCQEPHRFGHTPVQARLKRLAKNNSIYVVANMGDKKPCNSHSTCPSNGHYQYTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 48 - protein, 253 aa, Bos taurus
MGACHFQGLLLLFLVGAPTLIMAQKLFCQKGTFMGIQEDATNMFNWTSEKVEACDNGTLCQETILLIKTAGTKTAILATKSCSLDGTPAITFIQHTAAPSLAAISYSNYCEDPFCNNREGLYDIWNIQETEEETKGTTSLHCPTCLALGSCLNAPSVACPNNTDRCYQGKLQVSEGNVNSLLEIKGCTSIIGCKLMSGVFKIGPLWVKETCPSMSISTRKIDNGATWLHTSVWKLKLLLMLLLLILGGSASGP

SEQ ID NO: 49 - protein, 216 aa, Bos taurus
MLRLSERNMKILFVAALVVGSVFFLLLPGPSAADEKKKGPKVTVKVYFDLRIGDEDIGRVVIGLFGKTVPKTVDNFVALATGEKGFGYKDSKFHRVIKDFMIQGGDFTRGDGTGGKSIYGERFPDENFKLKHYGPGWGSMANAGKDTNGSQVFITTVKTAWLDGKYVVFGKVIEGMDVVRKVESTKTDGRDKPLKDVTIADCGKIEVEKPFAIAKE

SEQ ID NO: 50 - protein, 334 aa, Bos taurus
MNPSFFLTVLCLGVASAAPKLDPNLDAHWHQWKATHRRLYGMNEEEWRRAVWEKNKKIIDLHNQEYSEGKHGFRMAMNAFGDMTNEEFRQVMNGFQNQKHKKGKLFHEPLLVDVPKSVDWTKKGYVTPVKNQGQCGSCWAFSATGALEGQMFRKTGKLVSLSEQNLVDCSRAQGNQGCNGGLMDNAFQYIKDNGGLDSEESYPYLATDTNSCNYKPECSAANDTGFVDIPQREKALMKAVATVGPISVAIDAGHTSFQFYKSGIYYDPDCSSKDLDHGVLVVGYGFEGTDSNNNKFWIVKNSWGPEWGWNGYVKMAKDQNNHCGIATAASYPTV

SEQ ID NO: 51 - protein, 609 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTCSALVELLKHKPKATEEQLKTVMENFVAFVDKCCAADDKEACFAVEGPKLVVSTQTALA

SEQ ID NO: 52 - protein, 342 aa, Bos taurus
LSLGGPACSITMNWLVWALLLCSSAMAHVHRDPTLDHHWDLWKKTYGKQYKEKNEEVARRLIWEKNLKTVTLHNLEHSMGMHSYELGMNHLGDMTSEEVISLMSSLRVPSQWPRNVTYKSDPNQKLPDSMDWREKGCVTEVKYQGACGSCWAFSAVGALEAQVKLKTGKLVSLSAQNLVDCSTAKYGNKGCNGGFMTEAFQYIIDNNGIDSEASYPYKAMDGKCQYDVKNRAATCSRYIELPFGSEEALKEAVANKGPVSVGIDASHSSFFLYKTGVYYDPSCTQNVNHGVLVVGYGNLDGKDYWLVKNSWGLHFGDQGYIRMARNSGNHCGIANYPSYPEI

SEQ ID NO: 53 - protein, 1661 aa, Bos taurus
MKPTSGPSLLLLLLASLPMALGNPMYSMITPNILRLESEETVVLEAHGGQGTIQVSVTVHDFPAKKQVLSNENTQLNSNNGYLSTVTIKIPASKELKSDKGHKFVTVVATFGNVQVEKVVLISLQSGYLFIQTDKTIYTPGSTVLYRVFTVDHKLLPVGQTVFITIETPDGIPVKRDSKSSQNQFGILTLSWNIPELVNMGVWKIKAYYEDSPQQVFSAEFEVKEYVLPSFEVQLEPEEKFYYIDDPDGLKVNIIARFLYGEQVDGTAFVIFGVQDGDRRISLTHSLTRVPINDGNGEAILKRQVLLNGVQPSRADALVGKSIYVSATVILQSGSDMVEAERTGIPIVTSPYQIHFTKTPKFFKPAMPFDLMVYVTNPDGSPARHIPVVTQGSNVQSLTQDDGVAKLSINTQNKRDPLTITVRTKKDNIPEGRQATRTMQALPYNTQGNSNNYLHLSVPRVELKPGETLNVNFHLRTDPGEQAKIRYYTYMIMNKGKLLKVGRQYREPGQDLVVLPLTITSDFIPSFRLVAYYTLINAKGQREVVADSVWVDVKDSCMGTLVVKNGGKEEKHHRPGQQITLKIEADQGARVGLVAVDKGVFVLNKKNKLTQRKIWDVVEKADIGCTPGSGRNYAGVFTDAGLTLKTSQGLETQQRADPQCPQPATRRRRSVQLMEKRMDKAGQYSSDLRKCCEDGMRDNPMKFPCQRRAQFILQGDACVKAFLDCCEYITQLRQQHSRDGALELARSDLDDDIIPEEDIISRSQFPESWLWTVIEDLKQADKNGISTKLMNVFLKDSITTWEILAVSLSDKKGICVADPYEVTVMQDFFIDLRLPYSVVRNEQVEIRAILYNYREAENLKVRVELLYNPAFCSLATAKKRHQQTITIPARSSVAVPYVIVPLKIGLHEVEVKAAVYNHFISDGVKKTLKVVPEGVRVNKTVAVRTLNPEHLGQGGVQREEVPAADLSDQVPDTESETKILLQGTPVAQMTEDAIDGERLKHLIQTPSGCGEQNMIGMTPTVIAVHYLDSTDQWEKFGLEKRQESLELIRKGYTQQLAFRQKSSAYAAFQYRPPSTWLTAYVVKVFALAANLIAIDSKDLCETVKWLILEKQKPDGIFQEDGPVIHQEMIGGFRDTREKDVSLTAFVLIALHEAKDICEAQVNSLGRSIAKAGDFLENHYRELRRPYTVAIAAYALALLGKLEGDRLTKFLNTAKEKNRWEEPNQKLYNVEATSYALLALLARKDYDTTPPVVRWLNEQRYYGGGYGSTQATFMVFQALAQYQKDVPDHKELNLDVSIQLPSRNSAVRHRILWESASLLRSEETKENERFTVKAEGKGQGTLSVVTVYHAKLKGKVSCKKFDLRVSIRPAPETVKKPQDAKGSMILDICTKYLGDQDATMSILDISMMTGFSPDVEDLKTLSTGVDRYISKYEMNRDSNKNTLIIYLDKVSHTVEDCLSFKVHQYFNVGLIQPGAVKVYSYYNLDETCIRFYHPDKEDGMLSKLCHKDTCRCAEENCFMHHTEKEVTLEDRLDKACEPGVDYVYKTRLIQKKLEDDFDEYIMVIENIIKSGSDEVQVKQERKFISHIKCREALKLKEGAHYLVWGVSSDLWGEKPKISYIIGKDTWVELWPEAEECQDEENQKQCEDLANFTENMVVFGCPN

SEQ ID NO: 54 - protein, 415 aa, Bos taurus
MKWRTILPQCCLLLVTCALPALESVPIDIDKTKVKTTQPVDSAKIEPPDTGLYYDEYLKQVIDVLETDSHFREKLQKADIEEIKSGRLSKELDLVSHHVRTKLDELKRQEVARLRMLIKAKLDSFQDTGMDHHALLKQFDHLNHLNPDKFESTDLDMLIKAATSDLEHYDKARHEEFKKYEMMKEHERREYLKTLSEEKRKEEESKFAEMKKKHENHPKVNHPGSKDQLKEVWEEADGLDPNDFDPKTFFKLHDVNSDGFLDEQELEALFTKELEKVYDPKNEEDDMVEMEEERLRMREHVMNEVDTNKDRLVTLDEFLKATEKKEFLEPDSWETLDHQQFFTEEELKEYENLISVQESKLKKKADELQKQKEDLQRQHDQLEAQKLEYHQVVQQMEQKKLQQISPSAPGGESKI

SEQ ID NO: 55 - protein, 295 aa, Bos taurus
MPLPLACLLLLLLSTHSALALKICSFNVRSFGESKKANCNAMDVIVKVIKRCDIILLMEIKDSSNRICPTLMEKLNGNSRKGITYNYVISSRLGRNTYKEQYAFLYKEKLVSVKQSYLYHDYQAGDADVFSREPFVVWFQSPYTAVKDFVIVPLHTTPETSVREIDELADVYTDVKRRWNAENFIFMGDFNAGCSYVPKKAWKDIRLRTDPKFVWLIGDQEDTTVKKSTNCAYDRIVLRGQNIVNSVVPQSNLVFDFQKAYRLSESKALDVSDHFPVEFKLQSSRAFTNSKKKKK

SEQ ID NO: 56 - protein, 183 aa, Bos taurus
MAPLVGLFLIWAGASVFQQLHPVNGGDIPDPGSKPTPPGMADELPTETYDLPPEIYTTTFLPRTIYPQEEMPYDDKPFPSLLSKANDLNAVFEGPACAFPFTYKGKKYYMCTRKNSVLLWCSLDTEYQGNWKFCTERDEPECVFPFIYRKKSYESCTRVHSFFWRRWCSLTSNYDRDKAWKYC

SEQ ID NO: 57 - protein, 591 aa, Bos taurus
AVMVAPMYGSPGGRLARAVTRALALALVLALLVGLFLSGLTGAIPTPRGQRGRGMPVPPASRCRSLLLDPETGQLRLVDGRHPDAVAWANLTNAIRETGWAFLELHTNGRFNDSLQAYAAGVVEAAVSEELIYMYWMNTVVNYCGPFEYEVGYCERLKNFLEANLEWMQKEMELNNGSAYWHQVRLTLLQLKGLEDSYEGSVAFPTGKFTVKPLGFLLLQISGDLEDLEVALNKTKTNHAMGSGSCSALIKLLPGQRDLLVAHNTWHSYQYMLRIMKKYWFQFREGPQAESTRAPGNKVIFSSYPGTIFSCDDFYILGSGLVTLETTIGNKNPALWKYVQPTGCVLEWMRNVVANRLALDGDSWADIFKRFNSGTYNNQWMIVDYKAFVPGGPSPGRRVLTVLEQIPGMVVVADRTSELYQKTYWASYNIPSFESVFNASGLPALVARYGPWFSYDGSPRAQIFRRNHSLVHDLDSMMRLMRYNDFLHDPLSLCKACTPKPNGENAISARSDLNPANGSYPFQALHQRSHGGIDVKVTSTALAKALRLLAVSGPTWDQLPPFQWSTSPFSGMLHMGQPDLWKFSPIEVSWD

SEQ ID NO: 58 - protein, 444 aa, Bos taurus
MLPSKLHALFCLCTCLALVYPFDWQDLNPVAYIESPAWVSKIQALMAAANIGQSKIPRGNGSYSVGCTDLMFDYTNKGTFLRLYYPSQDDDHSDTLWIPNKEYFLGLSKFLGTHWLVGKIMGLFFGSMTTPAAWNAHLRTGEKYPLIIFSHGLGAFRTIYSAIGIDLASHGFIVAAVEHRDGSASSTYYFKDQSAVEIGNKSWLYLRTLKRGEEEFPLRNEQLRQRAKECSQALSLILDIDHGRPVMNVLDLEFDVEQLKDSIDRDKIAIIGHSFGGATVIQTLSEDQRFRCGIALDAWMFPVGDEVYSRIPQPLFFINSERFQYPSNIIRMKKCFLPDRERKMITIRGSVHQNFVDFTFATSKIIGYLFTLKGDIDSNVAISLSNKASLAFLQKHLGLQKDFDQWDSLVEGEDHNLIPGTNINTTNHQATLQNSTGIERPNLD

SEQ ID NO: 59 - protein, 148 aa, Bos taurus
MVMVLSPLFLVFILGLGLTPVAPAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRH

SEQ ID NO: 60 - protein, 757 aa, Bos taurus
MSRLFLACLLAIFPVVSMKSPIFGPEEVSSVEGRSVSIKCYYPPTSVNRHTRKYWCRQGAQGRCTTLISSEGYVSDDYVGRANLTNFPESGTFVVDISRLTHKDSGRYKCGLGISSRGLNFDVSLEVSQDPAQASHAHVYTVDLGRTVTINCPFTRANSQKIKSLCKKTNQDCFQVIDSNNYVSDKYKDRIHLSIPGTNTLVFSVVINRVKLSDAGMYVCQAGDDAKADKINIDLQVLEPEPELVYGDLRSSVTFDCSLGPEVAHVPKFLCQKKNGGACNVVINTLGKKAQDFQGRIVSVPKDNGVFSVHITSLRKEDAGRYVCGAQPEGEPQDGWPVQAWQLFVNEETAIPASPSVVKGVRGGSVTVSCPYNPKDANSAKYWCHWEEAQNGRCPRLVESRGLMKEQYEGRLVLLTEPGNGTYTVILNQLTDQDAGFYWCVTDGDTRWISTVELKVVQGEPSLKVPKNVTAWLGEPLKLSCHFPCKFYSFEKYWCKWSNRGCSALPTQNDGPSQAFVSCDQNSQVVSLNLDTVTKEDEGWYWCGVKEGPRYGETAAVYVAVESRVKGSQGAKQVKAAPAGAAIQSRAGEIQNKALLDPSFFAKESVKDAAGGPGAPADPGRPTGYSGSSKALVSTLVPLALVLVAGVVAIGVVRARHRKNVDRISIRSYRTDISMSDFENSRDFEGRDNMGASPEAQETSLGGKDEFATTTEDTVESKEPKKAKRSSKEEADEAFTTFLLQAKNLASAATQNGPTEA

SEQ ID NO: 61 - protein, 781 aa, Bos taurus
MAAHRCALLGALVLALGALSQPARLATPARPGATQARAPQGRVTEARPGSMVVEHPEFLKAGKEPGLQIWRVEKFDLVPVPPNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNDIYQWCGSSSNRFERLKATQVSKGIRDNERSGRARVHVSEEGAEPEAMLEVLGPKPALPAGTEDTAKEDAANRKLAKLYKVSNGAGTMSVSLVADENPFAQGALRSEDCFILDHGKDGKIFVWKGRQANTEERKAALKTASDFISKMDYPRQTQVSVLPEGGETPLFKQFFKNWRDPDQTDGPGLSYLSSHIANVERVPFDAATLHTSTAMAAQHGMDDDGRGQKQIWRIEGSDKVPVDPATYGQFYGGDSYIILYNYRHGGRQGQIIYNWQGAQSTQDEVAASAILTAQLDEELGGTPVQSRVVQGKEPAHLMSLFGGKPMIIYRGGTSREGGQTAPASTRLFQVRASSSGATRAVEVMPKAGALNSNDAFVLKTPSAAYLWVGAGASEAEKTGALELLRVLRAQPVQVAEGSEPDSFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDNYWSVDPLDRALAELAA

SEQ ID NO: 62 - protein, 874 aa, Bos taurus
MQSTLNLSTEEPVKRNTVKKYKIICIVLLILLVAVSLALGLVAGLRQQEEQGSCRKKCFDASHRGLEGCRCDVGCKGRGDCCWDFEDTCVQSTQIWTCNKFRCGETRLESSLCSCSDDCLQRKDCCADYKSVCQGETSWVDEDCSTAQQPQCPEGFDLPPVILFSMDGFRAEYLQTWSTLVPNINKLKTCGVHSQYLRPAYPTKTFPNHYTIVTGLYPESHGIIDNNMYDINLNKNFSLSSKEKDNPAWWQGQPIWLTAMYQGLKVGTYFWPGSDVAINGTFPSIYKIYNRSVTYEERIFTLLKWLDLPKAERPDFYTIYVEEPDSQGHNYGPVSAGVIQALQLVDKTFGLLMEGLKQRNLVNCVNIILLADHGMDQTYCDKLEYMADYFSSINFYMFEGPAPRIRTRNIPQDFFTFNSEEIVRNLSCRKPDQHFKPYLSPDLPKRLHFAKNVRIDKVNLLVDRQWQAVRNRAYSYCGGGNHGYDNEFKSMEAIFLAHGPSFKQKTEVEPFDNIEVYNLLCDLLHIQPAPNNGTHGSLNHLLKVPFYEPSHAEELSKFSVCGFTVPLPTDTLGCSCSRLQTNSDLERVNQMLDLTQDEITATEKLNLPFGRPRLIQKNKEPCLLYHREYVSGFDKTLRMPLWSSYTVPKPGDTSPLPPTVPDCLRADVRVAPSESQNCSFSLADKNITHGFLYPPANNRTSNSQYDALITSNLVPMYEAFKTMWNYFHSVLLVKYAMERNGVNVVSGPVFDYDYDGHFDAPDEIADYAVNTSVPIPTHYFVVLTSCKNQSQTPDACTGWLDVLPFVIPHRPTNVESCPENKSESLWVEERFNVHTARVRDVELLTGLDFYQEKAQPVSEILQLKTYLPVFETVI

SEQ ID NO: 63 - protein, 151 aa, Bos taurus
AAMRFLTVAFLFLALSASALAEPVKFKDCGSWVGVIKEVNVSPCPTQPCKLHRGQSYSVNVTFTSNTQSQSSKAVVHGIVMGIPVPFPIPESDGCKSGIRCPIEKDKTYNYVNKLPVKNEYPSIKVVVEWELTDDKNQRFFCWQIPIEVEA

SEQ ID NO: 64 - protein, 562 aa, Bos taurus
MDFLLALLLGSSLYLQAAAEFDGRWPRQIVSSIGLCRYGGRIDCCWGWARRPWGQCQPVCQPQCKHGECVGPNKCKCHPGFAGKTCNQDLNECGLKPRPCKHRCMNTFGSYKCYCLSGYMLLPDGSCSSALTCSMANCQYGCDVVKGQIRCKCPSPGLQLAPDGRTCVDIDECATGRASCPRHRQCVNTFGSYICKCHKGFDLMYIGGKYQCHDIDECSLGQYQCSSFARCYNIHGSYKCKCKDGYQGDGLNCVYIPRVMIEPSGPIHVPKGNGTISKGDGGHGNWIPDVRSTRWPLKTPYMPPVMIDRPTSKPTARPTPKPIPEPTPPPPPPTELRTPPPATAPERPTTRLTTVAPVATTSPREITVDNKIQTDTQKPRGDVFIPRQPSNDLFEIFEIERGVSADDEAKDDPGVLIHSCNFDHGLCGWIREKDSDVHWEPVRDPAGGQYLTVSAAKGPLGRAARLLLPLGHLLHAGDLCLSFRHKVTGLHSGTLQVLVRKHGAHGAALWGRNGGHGWRQTHITLRGADIKSVIFKGEKRHGHTGEIGLDDVSLKKGHCSEE

SEQ ID NO: 65 - protein, 227 aa, Bos taurus
MRVAGAAKLVVAVAVFLLTFYVISQVFEIKMDASLGNLFARSALDAVVRSTKPPRYKCGISKACPEKHFAFKMASGAANVVGPKICLEDNVLMSGVKNNVGRGINVALVNGKTGELIDTRFFDMWGGNVAPFIEFLKAIQDGTIVLMGTYDDGATKLNDEARRLIAELGSTSITHLGFRDNWVFCGGKGIKTKSPFEQHIKNNKDTNKYEGWPEVVEMEGCIPQKQD

SEQ ID NO: 66 - protein, 426 aa, Bos taurus
MRLVICSLLCAGALGLCLAVPERTVRWCTVSNLEASKCYSFHDNMKKVLSVESPHVTCVKRTSYLDCIRAIAAHEADAVMVDGGLVYEAGLKPYNLKPVVAEFYGSKDDPQTHHYVVAVVKKGSNFQLNQLQGKKSCHTGLGWSAGWNIPMRMLLPSDWSQKAAAKFFAGSCVPCADQSNFPKLCQLCAGKGMDKCACSHHEPYFGYSGAFKCLQDGVGDVSFVRHLTVFENLANQADRDQYELLCRENTRRPVHEYKGCHLARIPSHAVVARSVDGKEDLIWELLSQAQEHFGKDKSAEFQLFYSPHGKDLLFTDAAIGFLRVPPKMDAKLYLGYEYFSVIQHLGRVSQDGKEQLGSKCVNTPMKGYYVVAVVKKSDANLTWNSLRGKKSCHTAVGTSAGWNIPMGFLYNQTGSCKLGKFFSQSC

SEQ ID NO: 67 - protein, 150 aa, Bos taurus
MALKSLVVLPLLVLVLLLVRVQPSLGKESAAAKFERQHMDSGNSPSSSSNYCNLMMCCRKMTQGKCKPVNTFVHESLADVKAVCSQKKVTCKNGQTNCYQSKSTMRITDCRETGSSKYPNCAYKTTQVEKHIIVACGGKPSVPVHFDASV

SEQ ID NO: 68 - protein, 302 aa, Bos taurus
VSTMVPVLLALLLLLGPAVSEETQAGNYSLSFLYTGLSKPREGFPSFQAVAYLNDQPFFHYNSEGRRAEPLAPWSQVEGMEDWEKESALQRAREDIFMETLSDIMDYYKDREGSHTFQGAFGCELRNNESSGAFWGYAYDGQDFIKFDKEIPAWVPLDPAAQNTKRKWEAEAVYVQRAKAYLEEECPGMLRRYLPYSRTHLDRQESPSVSVTGHAAPGHKRTLKCLAYDFYPRSIGLHWTRAGDAQEAESGGDVLPSGNGTYQSWVVVGVPPEDQAPYSCHVEHRSLTRPLTVPWDPRQQAE

SEQ ID NO: 69 - protein, 479 aa, Bos taurus
MFRAALWPPVLLLLQLLLLACAPGGEGAHDQDEICFLPGLAKQPSFRQYSGYLKGSGSKRLHYWFVESQKDPKSSPVVLWLNGGPGCSSLDGLLTEHSPFLIQPDGVTLEYNPYSWNLIANVLYLESPAGVGFSYSDDKSYATNDTEVAQSNFEALKDFFCLFPEYKGNELFLTGESYAGIYIPTLAVLVMQDPSMNLQGLAVGNGLSSYEQNDNSLVYFAYYHGLLGNRLWSSLQTHCCSQNQCNFHDNKEPECVANLQEVSHIVASSGLNIYNLYAPCAGGVPSHVRHEKDTVVVQDLGNIFTRLPLKRVWHQTLLRSGEKVHLDPPCTNTTAASNYLNDPHVRKALHIPEQLPRWDLCNFLVNIQYRRLYQSMCSQYLKLLSAQKYRILLYNGDVDMACNFMGDEWFVDSLNQKMEVQRRPWLVDYGESGEQIAGFVKEFSHIAFLTIKGAGHMVPTDKPQAALTMFSRFLNRQPY

SEQ ID NO: 70 - protein, 243 aa, Bos taurus
MRPLLALLLLGLATGSAPLDDNKIPSLCPGHPGLPGTPGHHGSQGLPGRDGRDGRDGAPGAPGEKGEGGRPGLPGPRGEPGPRGEAGPMGATGPAGECSVPPRSAFSAKRSESRVPPPSDAPLPFDRVLVNEQGHYDAVTGKFTCQVPGVYYFAVHATVYRASLQFDLVKNGESIASFFQFFGGWPKPASLSGGAMVRLEPEDQVWVQVGVGDYIGIYASIKTDSTFSGFLVYSDWHNSPVFA

SEQ ID NO: 71 - protein, 473 aa, Bos taurus
LPHPVMRSWVVGARLLLLLQLVLVLGAVRLPPCTDPRHCTDPPRYTPDWPSLDSRPLPAWFDEAKFGVFVHWGVFSVPAWGSEWFWWHWQGEKLPQYESFMKENYPPDFSYADFGPRFTARFFNPDSWADLFKAAGAKYVVLTTKHHEGYTNWPSPVSWNWNSKDVGPHRDLVGELGTAIRKRNIRYGLYHSLLEWFHPLYLRDKKNGFKTQYFVNAKTMPELYDLVNRYKPDLIWSDGEWECPDTYWNSTDFLAWLYNDSPVKDEVVVNDRWGQNCSCHHGGYYNCKDKFQPETLPDHKWEMCTSIDQRSWGYRRDMEMADITNESTIISELVQTVSLGGNYLLNVGPTKDGLIVPIFQERLLAVGKWLSINGEAIYASKPWRVQSEKNSVWYTSKGLAVYAILLHWPEYGILSLISPIATSTTKVTMLGIQKDLKWSLNPSGKGLLVFLPQLPPAALPTEFAWTIKLTGVK

SEQ ID NO: 72 - protein, 126 aa, Bos taurus
MHLSQLLACALLLALLSLRPSEAKPGAPPKVPRTPSGEEVAEPQAAGGGQKKGDKTPGGGGANLKDDRSRLLRDLRVDTKSRAAWTRLLHEHPNARKYKGGNKKGLSKGCFGLKLDRIGSMSGLGC

SEQ ID NO: 73 - protein, 209 aa, Bos taurus
MTIQLRASCLFLFFLAGFVQTNSNLEKMDCYKDVKGTIYDYDAFTLNGKEHIQFKQYAGKHVLFVNVATYCGLTAQYPELNALQEELKPFGLVVLGFPCNQFGKQEPGENSEILPGLKYVRPGGGYVPNFQLFEKGDVNGETEQKVFTFLKQSCPHPSWEPIMVRDIRWNFEKFLVGPDGIPVMRWFHRTPVSTVKTDILAYMKQFKTK

SEQ ID NO: 74 - protein, 102 aa, Bos taurus
MRLSVTALLVTLALCYYEANAIVCPTFAADLTEFFYFPDLLYRLSLAKYNAPPEAVAAKMEVKQCTDRFSVKNRLIITNILGKILLNCTVTDVKAVLNPSSA

SEQ ID NO: 75 - protein, 341 aa, Bos taurus
MNFNVTFHLSYKFRLLLLFTLCLTVVGWATSNYFVGAIQEIPKAKDLVASLSKGVILGKKGTVTDEAFVEKARLDNCPSVSPHLKGQTKLVFKPDLTLEEVQAKNPKVHRGRYHPEECRALQRVAILIPHRHREKHLLYLLEHLHPFLQRQQLDYGIYVIHQAGSKKFNRAKLLNVGYIEALKDEIWDCFIFHDVDLVPENDLNLYRCEDQPRHLVVGRNSTGYRLRYSGYFGGVTALSREQFFKVNGFSNNYWGWGGEDDDLRLRVELHRMKITRPLPEVGKYTMIFHKRDQGNEVNIGRMNLLHQVSRVWRTDGLSSCGYKLLSVNYNPLYVNISGFLV

SEQ ID NO: 76 - protein, 542 aa, Bos taurus
  UNSURE 184: Xaa is any amino acid
MRHLAAGSFLSLLRVLLLPLAPAPAQDSASTSTPGSPLSPTEYERFFALLTPTWKAETTCRLRATHGCRNPTLVQLDQYENHGLVPDGAVCSDLPYASWFESFCQFTQYRCSNHVYYAKRVRCSQPVSILSPNTLKEVDSSPEVPPPTTATSPKSSHVTESPNPGLKPWAAVCIRGEPTFLTQXLSLGGQEQGQEPKQEQGQEHKQERGQEHKQEEGQEQEEQEEEQEEEKQEGQGTEEALESVSRLQADPEPKFRSELVSSNPFSFTPRVREVESTPMLMENLQELIRSAQEMNEMNDVYDGETIWRSQSPGSLLQLPHVEALLTLCYSIVENTCVITPTAKAWQYLENEILGFGISVCDSLGRRHLAACTLCDFCSLKLEQCHSEANLQRQQCDSSHKTPFVSPLLASQSMTIGTQIGSLKSGRFYGLDLYGGLRMDFWCARLATKGCEDNRVASWLQTEFLSFQDGDFPTRICDTEYVQYPNYCAFKSQQCMMRNRDRKVSRMRCLQNETYTVLTPDKSEDLVLRWSQEFSTLTLGQAG

SEQ ID NO: 77 - protein, 883 aa, Bos taurus
MGPRCRNLSALLLLLQVSSWLCQEPEPCIPGFGAESYTFTVPRRNLERGRVLGRVSFEGCAGLPRTVYVSDDTRFKVHTDGVLTVRRPVHLHRPELSFLVHAWDSTHRKLSTKVTLEVSAHHHHHHSHHDSPSGTQTEVLTFPGPHHGLRRQKRDWVIPPISCPENEKGPFPKSLVQIKSNKEKETQVFYSITGQGADTPPVGVFIIERETGWLKVTQPLDREQIAKYILFSHAVSSNGQAIEEPMEIVITVTDQNDNKPQFTQEVFKGSVLEGALPGTSVMQVTATDIDDEVNTYNAAIGYKILAQDPMLPHNKMFTINKETGVISVLTTGLDRESFPTYTLMVQAADLNGEGLSTTATAVITVLDTNDNAPRFNPTTYVGSVPENEANVAITTLTVTDADDPNTPAWEAVYTVLNDNEKQFIVVTDPVTNEGTLKTAKGLDFEAKQQYILYVAVTNVAPFEVTLPTSTATVTVDVIDVNEAPIFVPPQKRVEVPEDFGVGLEITSYTAREPDTFMEQKITYRIWRDTANWLEINPETGAISTRAELDREDVDHVKNSTYTALIIATDNGSPPATGTGTLLLFLDDVNDNGPVPEPRTMDFCQRNPEPHIININDPDLPPNTSPFTAELTHGASVNWTIEYNDQERESLILKPKKTLELGDHKINLKLIDNQNKDQVTTLDVHVCDCDGIVSNCRKATAFAEAGLQVPAILGILGGILAFLILILLLLLLVRRRRVVKEPLLPPEDDTRDNVYYYDEEGGGEEDQDFDLSQLHRGLDARPEVTRNDVAPTLMSVPQYRPRPANPDEIGNFIDENLKAADSDPTAPPYDSLLVFDYEGSGSEAATLSSLNSSESDQDQDYDYLNEWGNRFKKLADMYGGGEDD

SEQ ID NO: 78 - protein, 119 aa, Bos taurus
MGWTMRLVTAALLLGLAVAVTGEEEDADLCVYEALPDNDAVLCKGLKVFYPELGNIGCMIVPECNNYRQKITTWPEPIVKFPQALDDAAYILVMVDPDAPSRSSPKARFWRHWLVSDIK

SEQ ID NO: 79 - protein, 1303 aa, Bos taurus
MGAASGRRSPPLLLPLLLLLLPPPPVILELDPALQPGNFPADEAGAQIFAASFNSSAEQVLFQSTAASWAHDTNITEENARLQEEAALLSQEFSEAWGQKAKDLFDPVWQNFTDPTLLRIIGAVRTLGPANLDLEKRQKYNSLLSNMSRIYSTAKVCFPNKTAPCWSLDPELTNILASSRSYTLLLYAWEGWHNAAGIPLKPLYQDFTALSNEAYKQDGFSDTGAYWRSWYDSPTFTEDLERLYQQLEPLYLNLHAYVRRALHRRYGDRYINLRGPIPAHLLGNMWAQSWENIYDTVVPFPDKPNLDVTDVMVQKGWNATHMFRVAEEFFTSLGLLPMPPEFWAESMLEKPSDGREVVCHASAWDFYNRKDFRIKQCTRVTMDQLSTVHHEMGHVQYYLQYKGQHVSLRRGANPGFHEAIGDVLALSVSTPAHLHKIGLLDQVTNDTESDINYLLKMALEKIAFLPFGYLVDQWRWGVFSGRTPPSRYNYDWWYLRTKYQGICPPVVRNETHFDAGAKFHVPNVTPYIRYFVSFVLQFQFHEALCKEAGHQGPLHQCDIYQSTQAGAKLRALLQAGSSRPWQEVLKDMVGSDNLDARPLLSYFQPVTQWLEEQNQQNGEVLGWPEYQWRPPMPDNYPEGIDLVSDEDEARKFVEEYDRRSQVVNEYAEANWNYSTDISTDNSKLLMEKNLQMANHTVKYGTWARKFDVTNFQNATMKRMIKKIQDLERAALPTKELEEYNQILLDMETVYSVASVCHENGTCLRLEPDLTNLMATSRNYDLAWAWKSWRDKVGRSILPYPQYVELTNKAARLNGYQDGGDSWRSMYEMPFLEEELEQLFQELQPLYLNLHAYVRRALHRHYGPDVINLEGPIPAHLLGNMWAQSWSNIYDLVAPFPSAPKMDATEAMIKQGWTPLRMFKEADNFFTSLGLLPMPPEFWNKSMLEKPTDGREVVCHASAWDFFNGKDFRIKQCTSVNMEDLVVAHHEMGHIQYFMQYKDLPVTFREGANPGFHEAIGDVLALSVSTPTHLHKINLLSSGDGGYEEDINFLMKMALEKIAFIPFSFLVDQWRWRVFDGSVTRENYNQEWWSLRLKYQGVCPPLARSQDDFDPGAKFHIPASVPYVRYFVSFVIQFQFHQALCQAAGHQGPLHKCDIYQSKEAGKLLADAMKLGFSQPWPEAMRLITGQSNMSAAAMMTYFKPLLDWLVTENGRHGEKLGWPQYNWTPNSARLEGPFVGSGRVNFLGLNLEEQQARVGQWVLLFLGVALLVATLGLTQRLFSIRHHSLRRPHRGPQFGSEVELRHS

SEQ ID NO: 80 - protein, 319 aa, Bos taurus
MVGVLLLLLLLPFLLYIAAPQIRKMLSSGVCTSTIQLPGKVAVVTGANAGIGKETAKELARRGARVYLACRDVQNGELVAREIQMMTGNQQVLVRKLDLADTKSIRAFAKRFLEEEKHLHILINNAGVMMCPYSKTADGFEMHMGVNHLGHFLLTHLLLEKLEESAPSRVVNVSSLAHLLGRIHFHNLQGEKFYQSGLAYCHSKLANILFTQELARRLKASGSGVTVYSVHPGTVNSELVRHSALMRWIWWIFSFFIKTPQQGAQTSLYCALTEGLEVLSGNHFSDCHVAWVSAQARNETVARRLWDVSCDLLGIPVDW

SEQ ID NO: 81 - protein, 322 aa, Bos taurus
AASAMARLGRLLSVLGLVLCGATGLGLSAPPAPTPKKPIIGILMQKCHNKNMRALGKYYIAASYVKFLESAGARVVPVRLDLKNEEYEKLFKSINGVLFPGGSVNLMRSGYARVAKMFYNLSIKSFGEGDYFPVWGTCLGFEELIYLVSGESLLTLTDTVGIKLPLNFSRGTLQSRMFQNFPADLLLSLAVEPLTAHFHKWSLSVMNFTKNEKLKAFFSILTTNTDGNIDFISTMEGYRYPIYGVQWHPEKAPYEWGQLRGISHAPNAVKAAFYLAEFFVAEARKSNHHFESDVEETKALIYQYRPTYTGNVSSFQQSYIFD

SEQ ID NO: 82 - protein, 545 aa, Bos taurus
MSRHSQDERLGLPQPPALLPLLLLLLAVAVPLSQAGVYYATAYWMPTEKTIQVKNVLDRKGDAYGFYNNSVKTTGWGILEIKAGYGSQSLSNEIIMFAAGFLEGYLTAPHMDDHFTNLYPQLIKKRSMLNKVQDFLTKQDQWTRENIKYYKSDPFWRHADYVMAQMDGLFAGATKRAVLEGKKPMTLFQIQFLNAIGDLLDLIPSLSPTKNSSLKFFKRWDMGHCSALIKVLPGFENIFFAHSSWYTYAAMLRIYKHWDFNIVDKDTSSSRLSFSSYPGFLESLDDFYLLSSGLVLLQTTNSVYNKTLLQHVVPQSLLAWQRVRVASMMANNGKQWAEVFSKYNSGTYNNQYMVLDLKKVNLNHSLDEGTLYIVEQIPTYVEYSEQTAVLRRGYWPSYNIPFHEKVYNWSGYPILVKKLGLDYSYDLASRAKIFRRDQGKVTDMESMKYIMRYNNYKQDPYSKGDPCNTVCCREDLNSHSPSPGGCYDTKVADIYLASKYKAYAISGPTVQGGLPVFHWSRFNKTLHEGMPEAYNFDFITMKPIL

SEQ ID NO: 83 - protein, 102 aa, Bos taurus; identical to SEQ ID NO: 74

SEQ ID NO: 84 - protein, 431 aa, Bos taurus
MSRHSQDERLGLPQPPALLPLLLLLLAVAVPLSQAGVYYATAYWMPTEKTIQVKNVLDRKGDAYGFYNNSVKTTGWGILEIKAGYGSQSLSNEIIMFAAGFLEGYLTAPHMDDHFTNLYPQLIKKRSMLNKVQDFLTKQDQWTRENIKYYKSDPFWRHADYVMAQMDGLFAGATKRAVLEGKKPMTLFQIQFLNAIGDLLDLIPSLSPTKNSSLKFFKRWDMGHCSALIKVLPGFENIFFAHSSWYTYAAMLRIYKHWDFNIVDKDTSSSRLSFSSYPGYWPSYNIPFHEKVYNWSGYPILVKKLGLDYSYDLASRAKIFRRDQGKVTDMESMKYIMRYNNYKQDPYSKGDPCNTVCCREDLNSHSPSPGGCYDTKVADIYLASKYKAYAISGPTVQGGLPVFHWSRFNKTLHEGMPEAYNFDFITMKPIL

SEQ ID NO: 85 - protein, 574 aa, Bos taurus
MAVLVLAVILTLLGFAILAILLTRWTRRKQNEIDISRYSSEQSAGLLDYEDDREDFSSPRSKRGRGFWNLYSTESDISYDNREGYNRNYIPSTISLVLPQESVSDTNDLKATPELLPGTVGPITGTIGPIMQFTAPIPGATGPIKLSQKTIVQTPGPIVQYTGPSAETSGTTSEATPHAVNGPAPQALTCPPSMLRGLPLAPIMISQRTSTRPEKSKVRQEPPTHVAVPIITPVPIVIGPVAAVDSSGKVTLSPMVIFPGYMDGELIKKSDSKAQILKSGSTTKFQSSREENKEELQKKISFTDSDEVVIAEHKRELSNFQDAELQKGKIEMEVKVKDSNAEIMKRQESQEKVRDMKMPREAAAQVEKSEAEIQQGQEAKVEKNEAATAQGQEAQVKKSEAKIPKGQEAQVEKSEAQVPQGQEAQVEEHKVQIPQGQDTQAEKSEAKIPKGQDVQVEKSEAQVPQGQEAQVEERKVEIPQGQDAQAEKSEAKIQKGQDVEVEKSETKASQGQEVQVEKSESKIPKGQEAQVEKSEAEVPQGQEAQVEKSKAEIPQGQDAQTKESEAKIPKGQGP

SEQ ID NO: 86 - protein, 83 aa, Bos taurus
MGFFQLLMKKKELIPLVFFMTVAAAGASSFAVYSLRKSDVILDRKRNPEPWETVDPTVPTKLVTINQEWKPIEELQKVRRATR

SEQ ID NO: 87 - protein, 1831 aa, Bos taurus
LFCVTLQGLLFLLLVLLGTLANKLNVPQVLLPFGREPGRVPFLLEAQRGCYTWHSTHHDAVTVEPLYENGTLCSQKAVLIAESTQPIRLSSVILAREIVTDHELRCDVKVDVINSIEIISRTRELYVDDSPLELMVRALDAEGNTFSSLAGMVFEWSIARDNESAIEELSSKIRILRYSEAEYSPPAYIAEMEKEEKQGDMILVSGIKTGAAIVKVRISEPFYKKVAAALIRLLVLENIFLIPSQDIYLLVGAYIKYRVAKMVQGRMTEVKFPLEHYTLELQDHRVSCNISVSGEVASLDEKTAMVTAVQLGHTNLIFVHKNVHMRSVSGLPNCTIYVVEPGFLEMSKIVGLGWVVSVYRCENVSFILCIFKIHYRKMASSDNLRIMYQFLREYFEEQLTTVNGSYHVVKALKDGVVLINASLASIVYQNKNIQPIKFPIKHQQEVKIYFPIKLTPNFLAFPHHPMGMLYRYKVQVKGGSGNFTWASSNETVAMVTTKGVVTAGQVRGNSTVLARDVQNPFRYGEIKIYVLRLNKMELLPFHADVEIGQIIEIPIAMYHVNKETKEAIAFTDCSHLSLDLNMDKQGVFTLLKEGIQRPGPAHCSSTHIAAKSLGHTLVTVSVTDYEEYLESSATFAAYEPLKALNPVEVALVTWHSVKEMIFEGGPQPWILEPSRFFLELSMEKTEKIRITQVRLPAKRKQNQYIYRVLCLDLGEQILTFQIGNHPGVLNPSPAVEVVQVRFLCAHPASMSITPVYKVPAGAQPCPLPQHNKQLIPVSSLRDTVLELAVFDQHRRKFDNFSSLMLEWKSLNETLAHFENYNSVEMVAKDDGSGQTRLHGHQILKVHQIKGTVLIGVNFVGYSEKKSPKVSGDCYELSNLPRSVAVELLLVDDVTVLPENATIYNHPDVKEIFSLVEGSGYFLVNSSEQDIVTITYLEAESSVQLVPLHPGILTLEVYDLCLAFLGPAMAHLTVSDIQELELDLIDKVEIGKTVLVTVRVLGSSKRPFQNKYFRNMELKLQLASAIVTLALMEEQDEYSENYILRAITVGQTTLVAIARDKMGRKFTSTPRQIEVFPPFRLVPEKMTLIPTNMMQVMSEGGPQPQSIIHFSISNQTVAVVNRRGQVTGKVVGRAVVHGTIQTVNEDTGKVIVFSQDEVQIEVVQLRAVRILAAATRLITATEMPVYVMGVTSTQTPFSFSSASPGLTFHWSMSKRDVLDLVPRHSEVFLQLPVENNFAMVVHTKAAGRTNIKVTVHCMNSSSGQFEGNLLELSDEVQILVFEKLQLFYPECQPEQILMSMNSQLKLHTNREGAAFVSSRVLKCFPNSSVIEEDGEGLLKAGSIAGTAVLEVTSTEPFGVNQTTITGVQVAPVTYLRMSSHPKLYMAQGRTLSAFPLGMSLTFIVQFYNSIGEKFHTHNTQLYLALNRDDLLLIGPGNRNYTYMAQAVNRGVTLVGLWDRRHPGMADYIPVSVEHAIEPDTKLTFVGDVICFHTYLLNQNGEPGIWMISADNILQTDIVTGVGVARSPGTATIFHDIPGLVKTYREVVVNASSRLTLSYDLKTYLTNTPNSAVFKLFITAGRNGINLKGSCTPNQALAITTLLPETLMLCHVQFSNTLLDIPASKVFHIHSDFSMEKGVYVCLIKVRPQSEELLQVLSTADTSVYGWATLVSERSKNGMQRILIPFIPAFYINQSELVLSHKKDVGELRVLGVDRVLEKLEVFPSSPVLMVSGHRHSSLTPGLAIYHVRVVNFTSLQQMASPIFINISCVLTSQSEAVRVRAMKAKSGAVCQRVDQCDGSGVLQKFAGSYQSLLFTLFVMLASTAFLFLGKES

SEQ ID NO: 88 - protein, 325 aa, Bos taurus
MEASAPGRARRGWRRARAAGSPLSRAAMVLVVSALVLWAPPSVGYLDRLPRSAQLPQDSVKIVGSPNIPVKVYVMLHQKSPHVLCVTQRLRNSELVDPSFQWHGPKGKIISENSTAQITSTGSLVFQNFEESMSGVYTCFLEYKPTVEEVIKNLQLKYVVYAYREPHYYYQFTARYHAAPCNSIYNISFEKKLLQILSKLVLDLSCEVSLLKSECHRVKMQRAGLQNELFFTFSVSSLDTEKGPKPCADHSCESSKRLSKAKNLIERFFNQQVEVLGRRAEPLPEIYYIEGTLQMVWINRCFPGYGMNILKHPRCPECCGLKKYK

SEQ ID NO: 89 - protein, 1242 aa, Bos taurus
MEYLLFSPKSTFDTWLNSSYTAWTIEIILAVLCGLGLFFLFLPCHQNNPSFPRPRKRGTSRKHRMDLRGRSRSRKRSGALKVCRDCLEELEGIRNLVLLLQSHVGSLLDKKSFHQLSWQDPPGVGQEAAPAGAHQPSMELLEEAVSTISPAPLTQDPVPLASTLSAEPQDRSNLEKIPFDTVAKSSPPGNSFSASTISAMANLGLFTSYPISFLSCWWDTTKALFFPTSSQSKSWKEDLSPHPTKAPSWGRNIDKHVEIGSPSFINPDVQKLLEILITKRAELRICKEGKKDWSFSEQMSPDYHLNTSGNMWKSLGTEQDITTPQSFWNVKDKPEQLPEPQELLYPESLRDHIEQKYSQLFWGLPSLHSESLVAAALVPGSSSQSQAPFVLFNGISTVFPVSIQPEIPLSLSQDPLLPCAGTQPQPFTQNLTVYQPLPMVQIQGQSYLPSPLLFRPTYSSPQMSTCGLYYLPFQNNPLYFIPTETQSLEYPLLQKQLKTEMLLPSVNKRSQEVFSQLIPNCPQDTGEPQAQRLVPGPHGDLIKSDLQKQHLQKKPIEDQLKGSRLRSIHLSLQLSSSQCQFLVTYQAQGKQGSWQLSAFRGKRSQNAQKMKFQLETDFSEGLRPCLKGISKDPSRSSFPKKFLQTNSEKESERYLLRPSKSDLLRPSKSDSRDYLLSGPDKKHLEKVLKAHLDKKLRQIDQGLFPVTVRRSWLAATHASKTHLHKETRTLASLKHWKPRINISHELSFLNPSIQQMLEAHIIRFRVRHRWDTSKQGSEPVNLKPSEAQPSSLPTSTFSLSAIWQPGPQSKTNVSKFLGKPQPRQGKQMITKASVPSLGSLLPAPIPVRWDIQRALGKISPGDSCGPSEAPLTGQKGRPPCQMPAPNLKGRTRQNMGLKSQSSRAKQKANEDPAWEVFLGPTVPVNSQTSNMNLRRSDEPVTKKTLPPPTNSVGQYSVEPHFKTQVASKFELGSKLESENQLQDFATNLLLQDPHTDVLLAADILASHRSSSQMEYASGDTPASQVPYDLKVSAQSTQEQQKFQKPKLKDPFKSQREMLAPNEEWKGNWRPKLGKYEERNSGRRASHTRVMSQSPQARETESLGFSPEKEQLLPESYLKKRMRHFLQCLGANKKGRPASAAAQCQEQIKDKFIVDGRIIDPEMIGTVVGQVLAEKLGFQQGTHASEVNHHREQLQALVGGHSHHHRTLCSSEQIKMLKTSAHSHQANPKDRSHLSKSRHTRDR

SEQ ID NO: 90 - protein, 342 aa, Bos taurus
MGPRGLPLLVATLAGCLFPARGCVICDPKVREALNSLEMDYLPGHLEANHHKKVMEKIKQALEDFKDLPIDEDSYMGVVDEATLEKSSWSLLKDMKRITDSDAKGELFVKEMLWMLHLAKNTFASYAAQFQKEVSSEAAFCPNKCGLMLQPLIWCSTCQKQVHACRKSKNCGEREVKVHHMEDMILDCELNWHKISQGLTDYSFYRVWKNNSETLVSKGKEPILTKTMVRPKDAGTYRCELGSIQSSPATIIYFHVTVLPKRIQEEIPSPNTETQDETALGEVALDRPHTTTDFEPQTPKPEQLLRRRLLGVLIWGLVVLTVGVLTAILFSRSGKVVDFIKS

SEQ ID NO: 91 - protein, 280 aa, Bos taurus
MKSRGAGCSARLLLTVGWLLLAGLQSTCGINVTAIQDPSLAREGEGEPEGDEEPENDSETENEPQAEAEDDSEGIGIREVILTSGCPGGESKCIVRVEECRGPVDCGWGRPISESLEHVRLACVHTSPENRFKYIWRLLRPDQQAIILANDSAILEVHRDTHPKAFECETLDNNEIVASIRFTIYTTTELQMKRASRPDTDAVLVFVLTIGVIICIFVIFVLIFIIINWTAVKDFWGAKASTTEIQSELSSMRYKDSTSLDQSPTDIPGHEDDALSEWNE

SEQ ID NO: 92 - protein, 305 aa, Bos taurus
MGLQPSPVLLASLGVGLLTLLGVALGAYLVRRSRRPPVTLLDPNEKYQLRLLDKTTVNHNTKRFRFALPTAHHVLGLPVGKHVYLSARIDGSLVIRPYTPVTSDEDQGYVDLVIKVYLKGVHPKFPEGGKMSQYLDSLKIGDVVEFRGPSGLLTYAGKGKFNIQPNKKAPPEARVARNLGMIAGGTGITPMLQLIRAILKDPEDPTQCFLLFANQTEKDIILREDLEELQARHPGRFKLWFTLDHPPEGWAYSKGFVSVDMIREHLPAPGEDVLLLLCGPPPMVQLACHPSLDKLGYSPKMRFTY

SEQ ID NO: 93 - protein, 180 aa, Bos taurus
MVGSGIPALGLLLLMQGSADGNGIQGFFYPWSCEGDVWDRESCGGQAAIENPNLCLRLRCCYRDGVCYHQRPDENMRRKHMWALGWTCGGLLFLITSICLFWWARRHDMLRLPWFLKGKCDLSRTVSLLSKDRTPSEKKTPSVGSIPPAAPTEGALDVSGGTEGEGTEGGEETEGGDEDD

SEQ ID NO: 94 - protein, 569 aa, Bos taurus
MESWGPGMPGPTLFLLLVLPPPPLSLGSFQHSGPGWKDLHHLSLNSGSFQHRQGPGTWHRKSRSREDKNSCHSSFDLYFILDMSGSVNNNWMNIYSFVEDLVKKFENPNLRMSFITFSTEGHTVMQLTSDRNEVHDGLIRLQNIVPTGDTYMQEGFKKAIEQIEQAHSGENKVHSMIICLTDGTLEEKTLKETVKEADKARQMGATVYCVGVKNFEEHQLIEIADSPYHVFAVNQGFKALKDIVDPLETKSCIEITSVEPSDICVGDEYELMISGKGFNNAKNEDEVICRFKFKGDCVSDEKANSVQDTSMICPGIKIENPDEAFSLEISLNNGISFINSDVSLTSKDCASTRINPLYFFALIPALLLLSLMFCCIWWLCHRRTLKEPPPVLSPERHPENTIPQPPTWSDPGPLARDQHEPEETCQMSCPTVIVPCGCQGGGMKRMEGKLDSLCDFVQRCNQMSVMWCPTRDMPSHCSVPHLGFGRVMAACCPMLNNPHPLPTPLSHTHAGSASPSTTAAHGSSTPHPCAPGPPPECSRSSLPLPGQPTEPLCHSSPHSPSSKAPKPQD

SEQ ID NO: 95 - protein, 574 aa, Bos taurus
MAVLVLAVILTLLGFAILAILLTRWTRRKQNEIDISRYSSEQSAGLLDYEDDREDFSSPRSKRGRGFWNLYSTESDISYDNREGYNRNYSPSTNSLVLPQESVSDTNDLKATPELLPGTVGPITGTIGPIMQFTAPIPGATGPIKLSQKTIVQTPGPIVQYTGPSAETSGTTSEATPHAVNGPAPQALTCPPSMLRGLPLAPIMISQRTSTRPEKSKVRQEPPTHVAVPIITPVPIVIGPVAAVDSSGKVTLSPMVIFPGYMDGELIKKSDSKAQILKSGSTTKFQSSREENKEELQKKISFTDSDEVVIAEHKRELSNFQDAELQKGKIEMEVKVKDSNAEIMKRQESQEKVRDMKMPREAAAQVEKSEAEIQQGQEAKVEKNEAATAQGQEAQVKKSEAKIPKGQEAQVEKSEAQVPQGQEAQVEEHKVQIPQGQDTQAEKSEAKIPKGQDVQVEKSEAQVPQGQEAQVEERKVEIPQGQDAQAEKSEAKIQKGQDVEVEKSETKASQGQEVQVEKSESKIPKGQEAQVEKSEAEVPQGQEAQVEKSKAEIPQGQDAQTKESEAKIPKGQGP

SEQ ID NO: 96 - protein, 271 aa, Bos taurus
MAAGQIAVLGLVLLSAQGGFGAGSRIWTNIDNDGSKTRLTCALNHSATEIVGHRWVKGGKVLKEDALPDLKTEYEVDSEDRSGQYSCIFLPEHAGRTDLEVKGPPSIKAVKKSEHATEGETVILVCKSDSFPPVTNWLWYKESESGDQVITNSTQSKFFVVSSESRTELHIPNVDLKEDPGKYVCNGTNLEGTSQAAITLRVRNRFAALWPFLGIVAEVLVLVTIIFIYEKRRKPDEVLDDEDIGSAPLKSSGNPLNDKDKDKNVRQRNSS

SEQ ID NO: 97 - protein, 2670 aa, Bos taurus
MAPPVWTLVLLLGAAWARKEKPPDWKPLARSSRENSILTQCDFEDDSRPLCDWSQATKDDGDWTRESRPFLASGTAPPGGYPSGEGYYLHMDSSAFHPGGVARLRSPPIWEQGPLCVRFAYFMFGLSWGAQLKLWLASGAKGKHPNLLWKHVNTQSPSWIPTAVTVPLGLVLPSRLTFEGVWGSTTYLDIALDAISIHRGSCNRVCMMQTCNFDTLKDLCGWSWISTASGAKWVQKKGPTGVQDVGPEGDFSSPGDGYYMLLDPKNAKASQKSVLLSPLIQSSGCLSLSFHYLLRGRSPGAGFTAYASVLGSIRKHTIFSGQPGPNWQPVSVNYTGQGQIQFTLMGVFAKIPEPAVAVDAISIAPCGESFPQCDFEDKDHPFCDWVQVLQDGGRWTQGSANTLIHGTGPFGIALSGESHFVYLEANKFSQEGQSYKLVSRPFCAPGAICVAFTYHMDGRGKGTKLRLLLGSPAGSPPSSLWERVGSQGPDWLNDSMTIPSGHQQPMQLMFEVERGSHAAFVVALGFIFIGQGTCQGPASTVLPPKPLGPPTGPSETPVTPEETSTTSSEISPVPTEKPVAATEKPTVPSETPTVPTEKPVVTAEKLTVPTKRPTVPTEKPTILPEEPTFLPEGPTIPTEKPTVPTEKTTIPSKKTTIPTKKTTIPTEKPTVPTEKTTIPAKKTTILTEKPTILTEEPTFLPEWPLFPSEWPPVPTEKPIVSTERTTIPTEKTTIPTEKPMVPTEKTTIPTEKATVPTEKTTIPTEKTTIPTEKVTVPTEKTTIPTEKATVPTKTTTISTEETTISTEKTTVPTEKTTIPTEKTTIPIEKVTVPTEKTTIPTEKTTISTEKPMVPTEKTTIPTEKTTIPTEKVTVPTEKTTIPTEKTTISTEKATVPTEKTTIPTEKTTISTEKATVPTEKTTIPTEKATVPTKTTTIPTEETTISTEKTTVPTKKTTIPTKKTTIPTEKPTVPTERPTPPMTPQPSPALVPTQPTVFTKPTTSVTPVTPATTTTPRPTPATCPPNAHYERCACPESCQSPKPTCGLLCKPGCVCNSGFLFHDSRCINASSCNCFYNENYYKSGTEWFSPNCTERCRCQPGSQIECQPYKCGTNTVCQLKHGQYRCHPYAGTATCFVYGDPHYFTFDGRYFNFMGKCTYLLAQPCGNSTGPEPFFRVTVKNEQRGLEGVSCLSKVSVTLSETTITLLKGRHTLVGGQRVTLPAIPSGGVFLTPSGRFVQLQTAFGLRVRWDGDQQLYVRVPSTYSSKLCGFCGNYDGDSSNDNQKPDGRPARDEKELGHSWQISEDEDQACQENQASLPSCDTDLKNTLSSPEHCGRLVVANGAFQACLPHLKVSSFFHNCKLDMCNFQGLQQILCAHMSALTETCQEAGYAVKPWRGPQFCPLACPPNSRYTLCAKLCPDTCHSTFLGMACQNRCVEGCECNQGFVLSGLQCVPRSECGCLDPTAGYFKIGERWFKPGCRQLCICEGTNRTRCVPWQCQAQEMCGQQDGTYGCHPQGSATCTVWGDPHYLTFDGALHHFMGTCTYILTRPCLLKSLENYFFVSATNEFRGGNLEASYVKAVQVQAFGIRVWMLKGRKVMLDGRQVALPLWFAHGRMTVRSSGSFILLYTDFGLQVRYDGYHLVEVTAPSSYAGRLCGMCGNYNNNSLDDNLQPDKRPAVNSVRLGASWKLNELSESGCFAADARPPRCLEKNATDPWSKNCDILVNPQGPFSTCHRVVSPQASFASCVYGQCGTKGDAFTLCRSLQAYASLCARAGQVLTWRSSTFCPLKCPSGSRYSPCADPCPGTCLSLSTPSYCPSSLPCAEGCECQRGHILSGTTCVPLSECGCTSPGGAYHPVGERWYTDNTCSRLCTCSIHNNISCLQTSCKPGQMCWPRDGLMRCRGAGMGVCQIQDRSQYISFDGSYHAVRGACTYVLAKTCHSTMDLPFFKISGKNGKQQDQAHTFYLRKVYVHVFNTLVTLKKDHVLINGTWVPLPATSQIRGVKVISRDGYMVLTISIGVEVKFDGSGFLEIEIPKAYYGKTCGMCGNFNGEEEDELLMPNDELAPDDITYVDSWQDKEIDPSCRDDDKAEEESEEPETTCQPADLERAKEQCQAVFQTPGWARCASRVILSPFLVRCTDSLCEFGGLSSTLCQSLQAFATACQARGIRPPIWRNSSFCPLDCPPNYVYTNCFPHCPPTCDNPEGRCKGSSGPSVCQEGCVCEPGYVLKERRCVPRSQCGCRDARGRFLPEGNAWFSNSCSQRCTWRGGIQCRPFACPEGSRCEINEDDKEICKPYKSERCTVYGDPTYRTFDGLGYRFQGRMTYYLVKTVDVLPTGVEPFIVEGRNKMYASHNPVFLHEIIVMVYGYTVQLQHELELVVNGQKVTAPYEPVDQLRVSLRSDRLFVITDFELVVSFNGKNNAVISLPLTYRKLVLGLCGNCDQNKRNDFMLPNGAVTQNLLAFGNSWEVKMTEGSLPRVSRAVQEEEVKEEAALGFLDVSGCHPEELQLINRTQACGVLADPEGPFAVCHQTVAPEPFLEHCVSDLCGTRDPKEQEGLRCQVLSGYASICQEAGATPASWRDHTHCDSPCLQNPCQNDGRCWEQGTHFTCECEPGYGGDRCMELQDVPPPEKPEASNFASILLPLLVPMVVIVPAAVTRGCISRRKGRR

SEQ ID NO: 98 - protein, 1305 aa, Bos taurus
MENFLFSLKSTFDIWLNSCSTYWVTDTILAFLFGLGLFFLFLPYLENNPSFPPPRKHGDIKPQTEPRRRRRSRKKNAAVKACRDCQKELEEVRDLTSLLQTHLGRFPEKGNFHQLSSQDAPGKVSKAAPAGAHQPHREETAPTVSPAPLTEQPLPQASTLSSDSVPSSFSVHSDSSLSASQIPEPFLPLDSLSPQPLAVSPTPPCSPDAVACPAPPTASSVPQPPAAMLNLSQGESVALPLGTVSHRSSLPSPWRPAISGLLHSSCPLSALSWWQGAEKTWSSSTVTHFESQQEHLSSHPAEASFWGDLTNRQVEPGSLSFVNPDVQKLLEILITKRVELKIWKKKGKEVEASFWIIKGKPEQLLGPERAPYAGTLGDHLQKTCSHLFWGLPFLHSESLVATVTVPGSPLELHPILFNKCSKTKPLQIPAKATSQLSLAQPLTSTVAQVQPLPLTPIMPQCQPSPRFQLETAAHISPSLPIQPSSSKPQIRYGEVSHATVQSKGQTSVPNAIQNLEHHFLIKQLESRKIIPSLVKRYQHVFSQVTHNHSQESRPSQAHISVATPFGDLIRPEVWEKLEHHLSKRFMQQQSDLPCRIQASQKVMQLQGEFPKLCEVQAKKGPSIPSAVLDKSSQDTQKMRSRSSARIPPEKDLCQDIEQSVGKIVKDLYVISANTTVTVPRVKPESQRELSPDRKHPEDLGVLMGMNTKQIQESPIPVGVHCLRLAANRIVDLSGESKAYQETENLDSSQNGEASMNTFHESLVLSPCVRQELEAYILKFRVTHMWGLLLKVFKFILSLNLRKAHRIPLPGSTLKATCESGDHSKAQSTKIFGKPPQPQAGKRMITAEAASPMVTAFSTPSWTSEETQGVMGGPLPGGILKPSEVPLTRQGDKPPSQTPMYTFVGRIWHNECIMSQSPMEQEAIEGDSGWKSTFEPCLLIKSQSNNMDLRRSQSQKFNKCFSFNTKSVASSLDLHFDAQFRKLECQGFTDGQKQAQGQAVGMLLQDCETGATDMFIAANNLASQESLSCSQTLSSGGTTNSQMLYDASSTGGSSQGQQEVLRWQPQRTCQSKKFVPTDGRGNYRRSKLGQRKKRLAEWRAYQARRMNHSGQKKESAESLSKSRQFTLKKGQVPLESHFRIWIKQLLQWIFPNKSKRPEEHLQQAQEPVQSRLTTEGRAAEAQVLMTAVGKILKEKMVLHYRPHASELHWCQGELWAPFGPRYCYHRFSSYKEQRRVVGDTSCHHQATPMGHSCSKNSEWTSARGIKWAVLPREPGPPLGRACQHRSGVTSVSSHSLHCPRHCLFQK

SEQ ID NO: 99 - protein, 299 aa, Bos taurus
MKSRGAGCSARLLLTVGWLLLAGLQSTCGINVTAIQDPSLAREGEGEPEGDEEPENDSETENEPQAEAEDDSEVSNKTIVKEVEFGMCTVTCGIGIREVILTSGCPGGESKCIVRVEECRGPVDCGWGRPISESLEHVRLACVHTSPENRFKYIWRLLRPDQQAIILANDSAILEVHRDTHPKAFECETLDNNEIVASIRFTIYTTTELQMKRASRPDTDAVLVFVLTIGVIICIFVIFVLIFIIINWTAVKDFWGAKASTTEIQSELSSMRYKDSTSLDQSPTDIPGHEDDALSEWNE

SEQ ID NO: 100 - protein, 293 aa, Bos taurus
MNFILFIFLSGVFTPEISGIDPPYEAEHPVPEHPVPEHPMPEQPVPEQPHTMEEENKEHTPEKTGDHYKDVKQYVFTTQNPNGTQSEISVRATTDLNFSLRNYKLFNETTTTAPPEIVSHETEGTQSPNEPAFWTMLAKAINATTSEDNQRDQFFHPIPNSDVNTTWEDNTARIQEAKLKLMLGISLMTLFLFVVLLAICSAMLYKMKMMKYKQACQTGEYSVNPELATLSYFHPSEGVSDTSFSKSAESSTFWGTTSSELKKSDTRSKSRMTDMTSTASEENEESDMIQSEE

SEQ ID NO: 101 - protein, 355 aa, Bos taurus
MEASAPGRARRGWRRARAAGSPLSRAAMVLVVSALVLWAPPSVVGYLDRLPRSAQLPQDSVKIVGSPNIPVKVYVMLHQKSPHVLCVTQRLRNSELVDPSFQWHGPKGKIISENSTAQITSTGSLVFQNFEESMSGVYTCFLEYKPTVEEVIKNLQLKYVVYAYREPHYYYQFTARYHAAPCNSIYNISFEKKLLQILSKLVLDLSCEVSLLKSECHRVKMQRAGLQNELFFTFSVSSLDTEKGPKPCADHSCESSKRLSKAKNLIERFFNQQVEVLGRRAEPLPEIYYIEGTLQMVWINRCFPGYGMNILKHPRCPECCDILILPCNYNIQDYYHCSVYLSTNDLLYFNLCHSM

SEQ ID NO: 102 - protein, 972 aa, Bos taurus
MAGFRSLLVLLLVFPSGCVGFRSPLSVFKRFKETTRSFSNECLGTTRPVIPIDSSDFALDIRMPGVTPKQSDTYFCMSVRLPMDEEAFVIDFKPRASMDTVHHMLLFGCNMPASTGNYWFCDEGTCTDKANILYAWARNAPPTRLPKGVGFRVGGETGSKYFVLQVHYGDISAFRDNHKDCSGVSLHLTRLPQPLIAGMYLMMSVDTVIPPGGKVVNSDISCHYKKYPMHVFAYRVHTHHLGKVVSGYRVRNGQWTLIGRQSPQLPQAFYPVEHPVDVSFGDILAARCVFTGEGRTEVTHIGGTSSDEMCNLYIMYYMEAKHAVSFMTCTQNVAPDIFRTIPPEANIPIPVKSDMVMMHGHHKETENKDKTSLLQQPKREEEGVLEQGDFYSLLSKLLGEREDVVHVHKYNPTVKAESESDLVAEIANVVQKKDLGRSDTRESAEQERGNAILVRDRIHKFHRLVSTLRPAESRVLSLQQPLPGEGTWEPEHTGDFHVEEALDWPGVYLLPGQVSGVALDPQNNLVIFHRGDHVWDGNSFDSKFVYQQRGLGPIEEDTILVIDPNNAAVLQSSGKNLFYLPHGLSIDKDGNYWVTDVALHQVFKLDPKSKEGPLLTLGRSMQPGSDQNHFCQPTDVAVDPDTGTIYVSDGYCNSRLVQFSPSGKFITQWGEASLESSPKPGQFRVPHSLALVPPLGQLCVADRENGRIQCFKTDTKEFVREIKHPSFGRNVFAISYIPGLLFAVNGKPYFEDQEPVQGFVMNFSSGEIIDVFKPVRKHFDMPHDIAASEDGTVYVGDAHTNTVWKFTSTEKMEHRSVKKAGIEVQEIKESEAVVETKMENKPASSELQKIQEKQKLVKEPGSGVPAVLITTLLVIPVVVLLAIALFIRWKKSRAFGDSERKLEASSGRVLGRLRGKGGGGLNLGNFFASRKGYSRKGFDRLSTEGSDQEKDEDASESEEEYSAPPPAPAPSS

SEQ ID NO: 103 - protein, 279 aa, Bos taurus
MSRCAALSAAVAAGVAALVAALLLLQPEDVGQGASLSMAETEALPKLREDFRMQNKSVFILGASGETGRVLLKEILEQGLFSKVTLIGRRKLTLDEEAYKNVNQEVVDFEKLDDYASAFQGHDVGFCCLGTTRKKAGAEGFVRVDRDYVLKSAELAKSGGCKHFNLLSSKGADKSSNFLYLQVKGEVEARVEELKFDRYSIFRPGVLLCDRQESRPGEWLVRKFFGSLPESWASGHSVPVITVVRAMLNNVVRPSDKKKELLDNKAIHDLGKADDSLKP

SEQ ID NO: 104 - protein, 747 aa, Bos taurus
MIRLMILLAGLRGLLTSRPDFQNSFLQIILPEKVQANTSESENSEVENKQVSYSISIDEKPYTVHLKQRFFLANNFMVYMYNQGSVNSQSSNIQGQCYYQGYIEGYPNSFVTLSTCSGLRGILQFENVSYGIEPLESAFEFQHLLYKLGNGKNEFTVLTENKEDTEQNPMDYNIFISEKPESTVSTDLIPLYLEMHIVVDKALFDYLGSDSMIVTNKIIEIIGLVNSMFAQLKISIVLSSLELWSDKNKISTAGEAEELLHRFLEWKNSYLTLRPHDIAYLFIFREYPNYVGAAFPGKMCVTGYSAGVALYPKEMTLEAVSVIVTQMLGLSLGISYDDPKKCRCSGAICIMSTKAVQSSGMKTFSDCSLRDFEHFISNVGAQCLQNKPQMQDNPTEICGNGRVEGNEACDCGSEEDCGPDSCCDPLTCLMKPETECSEGSCCDDCQFMEAGAVCRPTAHPECDIPEFCNGSSGMCPADITILNGQECEGGKAYCFDGGCQDIDARCESIFGKGSRNAPFACYEEIQSQTDRFGNCGKSGGKYTFCPWRSLICGRLICTYPLQTPSFVKSGSICDKFKISQLCVKVLYFHSSCMDANVVKSVNLCSDGEICVNRRCSEVGPYINRVNQCSAQCGGNGVCTSRMICNCTEGYAPPNCTTRARISPGAQDLLTKRTSEKGGEKQWLLAIYIALPVLVVATIIVASWKLSKKWFSKKEESQSSGSTSESSSQSDTVRSKSESSPQTNRSRN

SEQ ID NO: 105 - protein, 240 aa, Bos taurus
MGDLWLLLLLPLSLAAFHGVKGCLECDPKFIEEVKSLLGKLVPPEVPGRTHMLERQMKEMIRLSFKVSHRDKMLRVLAVQKVVDLRTWLKIELDKLSKEKWKGVFILQGRLLDIRKNLDSKLEKLLKKFSEVACSEDCVVTEGPILDCWTCLRITSRCFRGEYCEEDDPKKAESREIGLFLILLAEGVILGGVLLLFHFCISHQRKMKAIRRSLKTYLEKKLEELMGIKDEKEKDFRGRE

SEQ ID NO: 106 - protein, 397 aa, Bos taurus
MLLAALVLPLPIFSVRCIDPPVLDSMKLKGVIKRHYYPGDRIDYECYRGYFYVWPYLLSATCEPNGSWSRIEEACLRKSCPDPQVKHGKVVAPNDTFEWESEAHISCDEGYFLRGKPIIICNLIGENVSWSDTIPHCEKIYCGQPPKIKHGKHTNSYRNIFEYNELITYSCDPSNGPDEYSLVGQNKLICIEPNKWSSDPPLCKVVKCQRPFLEHGIMVSGSRDKFSYQSVVVFECLQGFYLNGSNLVFCGGNDTWEPEMPTCIKGYKPTHPTKTPLSKYPGYPNPSDEIPSLEDFEDLDAGIIALAILTAIVAVVVVCTCLYRCLFSGKKEEKDMKEKVKEVKEKEVRDMKEEEREKEEKQRKEEKEKEEKKEKERKEKEKERKEKEKEKKQKEKE

SEQ ID NO: 107 - protein, 737 aa, Bos taurus
MLSLLLLLTGLGQLASAGHHSETARLQITVPRKIETNTKDGSISETHVAYSIQIDKKTYTLPLEKQSFLDSNLLVYSYNKSRVLYPDSSFIKGHCFYQGYATEIPNSVVTLSTCSGLRGLLQFENVSYGIEPLESSTTYEHVIYPFRDKKSDFSPISADQSYKILVKSNKDSGVLLKRVLKIHVIIDKALYHYMGSEVALASEKIIYVFSLINTMYSQLQVSVILTSLEIWSDLNKISTDGDAHDVLQRFVSWREKSLLQSSHDLAYLLIYRDHPNYVGATYHGMACDPKFAAGLALYSKMITLEAFSVIFTQLLGIMLGLKYNNNIYECSCPGIACIMNPEAIRSHGIKFFSSCSIDEFKHTTSQPKFECLQNRTVPKVVPQGRQSICGNGVLEPPEQCDCGSIENCEHTKCCNPLNCVLIGNADCGSGPCCGKKTCMILDRGTECRRSTDPCDFPEFCNGISEFCVPDMRAADLQTCNNKSSYCFRGICQDRTKQCVTLFGKYAKESSYQCTEEVNYQDDDFGNCLGRTCFFHQHLCGKLVCHWTHSAIVSRNDIDVQYTYLGGHICMSANLRTRRYPDPTVAANGSMCDENKFCDNGACRAIHQYTQYQDCDPLTKCKGHGICNTRLHCHCDVGYVPPDCEEIALSPGGSIDDGFWNLEIDESKTAYLIKRRRASQKNNLMISFYVLLPFFILTAIIALKWNKMKIFWNKEGTISGGSPSEISNSNSSQSNRNH

SEQ ID NO: 108 - protein, 744 aa, Bos taurus
IGVGEFLVYMKVTLLLVWLKVFLFLPGWPQLGHAQRLGPPEVVIPWRVTPTGRGMKLQGWFSYKLHFGGQRHVVHIKVKKNFLSKNFPVFTYADKGVLLQDQPFVQDDCYYRGYVEGDPDSLVSLSNCFGGFQGMLQTNYIVYEIEPKRFSTAFEHLIYKLDNKDTNFSYFRCGLTDEKIAGQLKIQESINSTLMQSAYTGWWTHHYFLEVAVVIDYSRYLHHQSNASLVEKEVFLVLNGVSDLMKPLDLEVFFKGMEIWTQKSLIAIGGAGKTLDNFCKWKQKGFDKRVPHDVVHIFVKKNYGETLGLAFVGTVCQRQFSCGIETFHDQRIFILSYIVTHEMGHNLGMDHDNPKICKCGASECILFPSVALTTKFSNCSYADYCNLGHRRRCLYTSPNPHTVIRETRCGNRVVEEGEECDCGSLETCNTDPCCQLNCTMTAGINCAFGLCCHNCMFSQSGTVCRKVANECDLPEWCNGTSSQCPDDVYVQDGASCTGGGYCYGKRCNERDEHCRQIFGKEAKDADMSCYTAVNTRGDRFGNCGITETSYIRCSMADSLCGRIQCENVKEIPVMSDHITLHWTKFENNTCWGTDYHLGIRIADIGAVNDGTKCGPENICIRRRCVSYSTVPRNCSPQLCSLRGVCNNRQHCHCNYGWGPPVCQEKGRGGSVDSGPAPERQTEERMDRKNLLTYFLIPFLFLLLCLLLWLWLFTRPKKKDEKEQAVPPKSEKTTQNLPKKKKHRM

SEQ ID NO: 109 - protein, 449 aa, Bos taurus
LVPVRVSCIAFLFIFLWPVAALSTIGCRAQPTKPAKNWRRLAQPTLKFLFQVTFFLAGFLVKVKGKKATRDEARIFVAAPHSSFFDAIACVVAGLPSVVSASQNANIPVAGKFLLSTQPVLVTRDDPNSRKTTREEILKRVTSNRQWPQILIFPEGVCTNRSCLVTFKLGAFSPGVPVQPVLLRYPNPLDTVTWTWQGFTGFQACMLTLSQPFTRVEVEFMPVYIPNVQEKKDPVLFANTVRIIMANALGVPVTDHTYEDCRLMISAGNLQLPMEAGLVEFTKISQNLKLDWDNIHQCLDKYAEIAVASKGGKIGIEEFANYLKLPISKPLQQLFALFDRNNDGTIDFREYVIGLTVLCNPVNTEKILQMSFKLFDLDKDGFITEQELAAILRAAFGVPDLDVSTLFREIAGPNSDRISYRTFKKFALKHPAYDVKVSPENGESNIQVK

SEQ ID NO: 110 - protein, 150 aa, Bos taurus
PRMTSPLLISLASCLVAVNQASLIGRCDLAKVLHQEDLDGFEGYSLTDWLCLAFVESDFNITKVNENTDGSFDYGIFQINSHYWCNDYQSHTENNCQVDCQELLSPNLLAIINCAKKIVSGAGGMKNWVKWRLHCAGRPLSYWMTGCHLA

SEQ ID NO: 111 - protein, 244 aa, Bos taurus
MALLPVVLFLTAVLLPSFPTEGKDPSFTALITTQTQVQREIVNKHNELRKSVSPPASNMLKMEWSREVTANAQKWANKCTLEHSSPNERKTSTKCGENLYMSSDPTAWSDAIQSWFNEHHDFIYGSGPKSTGAIVGHYTQLVWYSSFRVGCGIAYCPNQESLKYYYVCQYCPAGNNVSKKNTPYQQGAPCGSCPGNCDNGLCTNTCEYEDLLSNCDSLKKTAGCEHELLKEKCKATCLCEGKIY

SEQ ID NO: 112 - protein, 327 aa, Bos taurus
MRALLPLLRTSWAWPVGAAAAASARLLRGASAGPGPRRTMALYRTEERGQPHSPDYRLFFKNVAGHYISPFHDIPLKVDSEENVFNMVVEVPRWTNAKMEIATKEPLNPIKQDVKDGKLRYVANIFPHKGYIWNYGALPQTWEDPHRKDKSTDCCGDNDPIDVCEIGSKVLSRGEVVHVKILGVLALIDQGETDWKLIAINVNDPEASKFHDIEDVKKYKPGYLEATLNWFRFYKIPEGKPENQFAFNGEFKNKAFALEVIKSAHECWRALLMDKCDGGAINCTNVQVCDSPFHCSQEEARSLVESVSFSLNKGSNEEDQVWHFLGK

SEQ ID NO: 113 - protein, 1426 aa, Bos taurus
RALSFGLELASWVLSYFSLLFDFLRSLLSSILDMPSSLLGSAMPASTSAAALQEALENAGRLIDRQLQEDRMYPDLSELLMVSAPNNPTVSGMSDMDYPLQGPGLLSVPNLPDISSIRRVPLPPELVEQFGHMQCNCMMGVFPPISRAWLTIDSDIFMWNYEDGGDLAYFDGLSETILAVGLVKPKPGIFQPHVRHLLVLATPVDIVILGLSYTNLQTGSGVLNDSMCGGMQLLPDPLYSLPTDNTYLLTITSTDNGRIFLAGKDGCLYEVAYQAEAGWFSQRCRKINHSKSPLSFLVPSLLQFTFSEDDPIVQIAVDNSRNILYTRSEKGVIQVYDLGHDGQGMNRVASVSQNSIVSAAGNIARTIDRSVFKPIVQIAVIENSESLDCQLLAVTHAGSGVRLYFSTCPFRQPLARPNTLTLVHVRLPPGFSASSTVEKPSKVHKALYSKGILLMAASENEDNDILWCVNHDTFPFQKPMMETQMTTRVDGHSWALSAIDELKVDKIITPLNKDHIPITDSPVVVQQHMLPPKKFVLLSAQGSLMFHKLRPVDQLRHLLVSNVGGDGEEIERFFKLHQEDQACATCLILACSTAACDREVSAWATRAFFRYGGEAQMRFPTTLPTPSNVGPILGSPVYSSSPVPSGTLYPNPSFLGTPSQGVHPPAVSTPVCALGSPATQATSMSCMAGPEIVYSGKHNGICIYFSRIMGNIWDASLVVERVFKSGNREITAIESSVPSQLLESVLLELKGLQEFLDRNSQFTGGPLGNPNTAAKVQQRLIGFMRPENGNTQQMQQELQRKLHEAQLSEKISLQAIQQLVRKSYQALALWKLLCEHQFTVIVGELQKEFQEQLKITTFKDLVIRDKELTGALIASLINCYIRDNAAVDGISLHLQDICPLLYSTDDAICSKANELLQHSRQVQNKIEKERMLRESLKEYQKISNQVDLSSVCAQYRQVRFYEGVVELSLTAAEKKDPQGLGLHFYKHGEPEEDIVGLQAFQERLNSYKCITDTLQELVNQSKAAPQSPSVPKKPGPPVLSSDPNMLSNEEAGHHFEQMLKLSQRSKDELFSIALYNWLIQADLADKLLQIASPFLEPHLVRMAKVDQNKVRYMDLLWRYYEKNRSFSSAARVLSKLADMHSTEISLQQRLEYIARAILSAKSSTAISSTAADGEFLHELEEKMEVARIQLQIQETLQRQYSHHSSVQDAISQLDSELMDITKLYGEFADPFKLAECKLAIIHCAGYSDPILVQTLWQDIIEKELNESVTLSSPDRMHALSLKIVLLGKIYAGTPRFFPLDFIVQFLEQQVCTLNWDVGFVIQTMNEIGVPLPRLLEVYDHLFKSRDPFWNRMKKPLHLLDCIHVLLTRYVENPSQVLNCERRRFTNLCLDAVCGYLVELQSMSSSAAVQAITGNFKSLQAKLERLH

SEQ ID NO: 114 - protein, 431 aa, Bos taurus
MPCPRLLAALCGALFCSSGLFAFSGDFCDSSQCLHGGTCLLNEDRTPPFYCLCPEGFTGLLCNETEHGPCFPNPCHNDAECQVTDDSHRGDVFIQYICKCPLGYVGIHCETTCTSPLGMQTGAIADSQISASSMHLGFMGLQRWAPELARLHQTGIVNAWTSGNYDKNPWIQVNLMRKMWVTGVVTQGASRAGSAEYLKTFKVAYSTDGRQFQFIQVAGRSGDKIFIGNVNNSGLKINLFDTPLETQYVRLVPIICHRGCTLRFELLGCELNGCTEPLGLKDNTIPNKQITASSYYKTWGLSAFSWFPYYARLDNQGKFNAWTAQTNSASEWLQIDLGSQKRVTGIITQGARDFGHIQYVAAYRVAYGDDGVTWTEYKDPGASESKIFPGNMDNNSHKKNIFETPFQARFVRIQPVAWHNRITLRVELLGC

SEQ ID NO: 115 - protein, 239 aa, Bos taurus
MGIFLAYVGFVFFSVLYVQQGLSSQAKFTEFPRNVTATEGQNVEMSCAFQSGSASVYLEIQWWFLRGPEDLEPGADVAGAQAELLPDRDPDGDGTKISTVKVQGNDISHKLQISKVRKKDEGLYECRVTDANYGELQEHKAQAYLKVNANSHARRMQAFEASPMWLQDMKPRKNVSVAAPSSMHSSANQRVPSTSSPQEVAKIPKQSPQSAKSKSPVKSTERTAKLTLNSKHHSAPTVL

SEQ ID NO: 116 - protein, 246 aa, Bos taurus
MKTFIFLALLGAAVAFPVDDDDKIVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN

SEQ ID NO: 117 - protein, 789 aa, Bos taurus
MLGARALRCRGFLLPGAGPVLALSYRGSSARDKDRSVTVSSSVPMPAGGKGSRTNCSQSTPQKVPNRLINEKSPYLLQHAYNPVDWYPWGQEAFDKAKKENKPIFLSVGYSTCHWCHMMEEESFQNEEIGRLLSEDFVSVKVDREERPDVDKVYMTFVQATSSGGGWPMSVWLTPDLQPFVGGTYFPPEDGLTRVGFRTVLMRIRDQWKQNKSTLLENSQRVTTALLARSAISMGDRQLPPSAATMNSRCFQQLDEGYDEEYGGFAEAPKFPTPVILSFLFSYWLSHRLTQDGSRAQQMALHTLKMMANGGIRDHVGQGFHRYSTDRQWHVPHFEKMLYDQAQLTVAYSQAFQISGDEFYSEVAKGILQYVVRNLSHRSGGFYSAEDADSPPERGMRPKEGAFYVWTVKEVQHLLPEPVLGATEPLTSGQLLMKHYGLTEAGNISPSQDPKGELQGQNVLTVRYSLELTAARFGLDVEAVRTLLNSGLEKLFQARKHRPKPHLDSKMLAAWNGLMVSGFAVTGAVLGQERVINYAINGAKFLKRHMFDVASGRLMRTCYAGSGGTVEHSNPPCWGFLEDYAFVVRGLLDLYEASQESAWLEWALRLQDTQDRLFWDSRGGGYFCSEAELGAGLPLRLKDDQDGAEPSANSVSAHNLLRLHGFTGHKDWMDKCVCLLTAFSERMRRVPVALPEMVRALSAHQQTLKQIVICGDPQAKDTKALLQCVHSIYIPNKVLILADGDPSSFLSRQLPFLNTLRRLEDRATAYVCENQACSMPITEPCELRKVLHQ

SEQ ID NO: 118 - protein, 345 aa, Bos taurus
GVTVTPDEEQNLNHYVQVLQNLILSVPTKEPGRQKKSKSPNNANFIGPRVSRVKELKYTHDVGPGDNDVLINPVSEETTTFPTRGFTLEIDKKKRTKSTAFWSIKPSNVSVVLHAKEPFIEKDEPEPEPEPEPEPEPVEHRTGAPTQVPSVTEPSQDVTSLSGSTDLGTATEEEDVPQLSGDNEMDYLESHDMYNEDVLKRIADINSQLHHVPLPESYKPEYRADIRASKEHLKRSLALAIAAEHKLEKMYKSQMLPQGRSSGGVYDIITVINMLYNSRYKLSEYLDIKYVPLEMRGKATVVVHTLKRILCVGHGEETHNLLKQLLNNNIRILHILDTHDKDDSS

SEQ ID NO: 119 - protein, 238 aa, Bos taurus
MHSVWRLLVLLGLLALPSALHKPHQPGLAKTHTDSKSALARIIAQGLMKHNTEGRIQNIHLLDSLNASGKMAPGMVGWLIGSMSLHHHQEGSANITNAQLDYGGIRMSFHKEWFTANISLGFDIDLRLPFNNHIIKTHERMNLSVEFWLEKDEFGRRDLVIGKCHVEPRSVHTTFLTEAIPPKVNPFLRNFRDNLEKVIPHLIESQVCPLIDEILRQLDVKLLKSLMEQDIAHKLNQL

SEQ ID NO: 120 - protein, 869 aa, Bos taurus
MLPLSRWLRSVGVFLLPAPCWVPQDRLLGPLQRPTLGHGCPVLGAWHSARCWRQAWTEEPRAFYSSLRMNGDQKLDAYAQERQDFIQHFSQIVKVLTEEDIGHPEIGDAITRLKEVLEYNAIGGKYNRGLTVVITFRELVEPGKQDPDSLQRALTVGWCVELLQAFFLVSDDIMDSSLTRRGQTCWYQKPGIGLDAINDAFLLESSIYRLLKLYCREQPYYLDLIELFLQSSYQTEIGQTLDLITAPQGNVDLGRFTEKRYKSIVKYKTAFYSFYLPVAAAMYMAGIDGEKEHAHAKKILLEMGEFFQIQENYGQKEAEKVARVKALYEEMNLSAVYMQYEEDSYNHIMGLIEQYAAPLPPAIFLGLAQKIYKRKNSCSDLSPDESPISVYSRDLPGNEDVHPQPSIVPLEQGSPLASAVRSSWSFAGVPGAQRLWMAEAQSGTGQLQEQKKGLLIAVSASVDKIISHFGAARNLVQKAQLGDSRLSPDVGHLVLTTLCPALHALVADGLKPFRKDLITGQRRSSPWSVVEASVKPGSSTRSLGTLYSQVSRLAPLRSSRSRFHAFILGLLNTKQLELWFSSLQEDAGLLSLLYLPTGFFSLARAGCPSLSTELLLLLQPLSVLTFHLDLLFEHHHHLPLGPPQAPPPPGSPPALQQTVQAVLHWGGRLAQSLRGASGEAPPGPSAPSSSPSPCSWWEQLTQASRVYASGGAEGLPLPRWGSRRTQTAAEATQERSPHTEEAAPGRGVWLGRLFGVPGGLAETESGAPKSRRPSSWLPPTVSVLALVKRAAPPEAPSSPKELEVSEPSTVQTHRAVRALCDHTAAGPDQLSFQRGEVLRVIATVDEDWLRCGRDGAEGLVPVGYTSLVL

SEQ ID NO: 121 - protein, 478 aa, Bos taurus
TVGAMWKALFLLVLLVLSPGADGLFRSLYRKGHVSSPRKGDPGQRLFLTPYIETGKLEEVGRQLSLVPPFPGWNLTSYSGYITVNKTYNSNIFFWFFPAKIEPQNAPVVLWLQGGPGGSSMFGLFVEHGPYIVSKNMTLFARDFPWTITFSMLYVDNPVGTGFSFTDHVHGYAIDEDDVARNLYSALIQFFELFSDYRDNDFYVTGESYAGKYVPAIAHYIHILNPVTTMKINLKGIALGDAYFDPKSIIEGYPSFLFQIGLLDEQEKKYFQKQCNDCVKFIRQEKWLQAFELLDRLLDGGLISEPSYFQNVTGCSNYYNLLLCTEPEDQGYYGKFLSLPHVRQAIHVGNQTFSDGAKVEKYLREDTVKSVKPWLTEIMNNYKVLIYNGQLDVIVAASLTERSLMAMDWKGSQKYKKAGKKVWKILKSDPEVAGYVRQVGNFCQVIVRGGGHILPYDQPLRSFDMINRFIFGRGWDPY

SEQ ID NO: 122 - protein, 154 aa, Bos taurus
MGPHLLGRSSLLLLLLGMWWSVRPLCAVPKGLTKARWFEIQHIQPRLLQCNKAMSGVNNYTQHCKPENTFLHNVFQDVTAVCDMPNIICKNGRHNCHQSPKPVNLTQCNFIAGRYPDCRYHDDAQYKFFIVACDPPQKTDPPYHLVPVHLDKVV

SEQ ID NO: 123 - protein, 542 aa, Bos taurus
MLCLLFLLLGLTGLQTDDNSERLRVQFTVPEKIRSTSSGGVETHVSYIILIEGKTYTVNLMQKAFLPHNFRVYGYSGTGSMKPLEHEFQNFCYYQGYIEGYPNSMAIISTCTGLRICRGLLQFENVSYGIEPLEPSIGFEHMVYQIKPRDSSSSVYTEREIELREKPYKIQNVEPLPDFSQYIEMHIVVEKDLYNHMGADTTVVIQKIFQLTGLTNAIFTSLNITVILSSLELWIDENKIPVTGDANELLHRFVKWKRSYLVLRPHDMAFLLVYREKSNYIGATFQGRMCDKHYGGGVALHSSTISLESLAVIIAQLLSLSMGIPYDDINKCHCPGDVCIMNPAAVHSSGVKLFSNCSVEDFLRFISKPKSQCLQNQPRLDPTYKSAVCGNGKVEEGEQCDCGNKKACDALPDTCCVADTCRFQPGSACDTGLCCESCAFIPKGHICRGSTDECDLHEYCNGSSAACQEDVYVQDGHPCGQNQWLCISGICVDGIKQCFDIFGEGTSYAPAECFQRLNSMNDLSGNCGVTPTGFTPCTSEYV

SEQ ID NO: 124 - protein, 148 aa, Bos taurus
MKAAGILALMGCLVTVVEPKVYTRCKLAKIFSRASLDNYRGFSLGNWICMAYYESHYNTTAQTQLEDGSTDYGIFQINSDTWCRSTKLQEKNRCHVACSALMTDDLTDAIICAKKIVKETDGMNYWQGWKKNCEGRDLSEWKKGCEVS

SEQ ID NO: 125 - protein, 421 aa, Bos taurus
MLPLSRWLRSVGVFLLPAPCWVPQDRLLGPLQRPTLGHGCPVLGAWHSARCWRQAWTEEPRAFYSSLRMNGDQKLDAYAQERQDFIQHFSQIVKVLTEEDIGHPEIGDAITRLKEVLEYNAIGGKYNRGLTVVITFRELVEPGKQDPDSLQRALTVGWCVELLQAFFLVSDDIMDSSLTRRGQTCWYQKPGIGLDAINDAFLLESSIYRLLKLYCREQPYYLDLIELFLQSSYQTEIGQTLDLITAPQGNVDLGRFTEKRYKSIVKYKTAFYSFYLPVAAAMYMAGIDGEKEHAHAKKILLEMGEFFQIQDDYLDLFGDPSMTGKIGTDIQDNKCSWLVVQCLQRASPEQRQILQENYGQKEAEKVARVKALYEEMNLSAVYMQYEEDSYNHIMGLIEQYAAPLPPAIFLGLAQKIYKRKK

SEQ ID NO: 126 - protein, 460 aa, Bos taurus
MAPWLQLLLLLGLLPAAAPASSKPPAAGAQAWELASPELREPARFALEMYNRGRAAGTRAALGAVRGRVRRAGRGSLYSLKATLVEPPCNDPTVCQLPVSKKTLLCSFEVLDELGKHMLLRRDCGPVDTKTTDDRNETFSSFLPLLNKDPLPQDFSVKMASIFKDFVTTYNRTYDSQEEASWRMSVFANNMVRAQKIQALDRGTARYGVTKFSDLTEEEFRTIYLNPLLKDAPGRNMRPAQPVTDVPPPQWDWRNKGAVTNVKDQGMCGSCWAFSVTGNVEGQWFLKRGTLLSLSEQELLDCDKTDKACLGGLPSNAYSAIRTLGGLETEDDYSYRGRLQTCSFSAEKAKVYINDSVELSKNEQKLAAWLAKNGPVSIAINAFGMQFYRHGISHPLRPLCSPWLIDHAVLLVGYGNRSAIPFWAIKNSWGTDWGEEGYYYLHRGSGACGVNIMASSAVIN

SEQ ID NO: 127 - protein, 349 aa, Bos taurus
MAGGFLGKTLAAVSLPVALASVTVKSLGSCSVAACRNSLSSCRFHVNSNIMSGFNGTKENSHNKARTSPYPGSKVQRSQVPNDKVDWLVEWHDYNPVEYTAVSVLAGPQWADPQITESNFSPKFNEKDGQVERKSQNGLYEVKNGRPRNPAGRTGLVGRGLLGRWGPNHAADPIITRWKRDRSGNKITHPISGKNILQFVAIKRKDCGEWAIPGGMVDPGEKISATLKREFGEEALNSLQKSSAEKRELQEKLQKLFNQEHLVIYKGYVDDPRNTDNAWMETEAVNYHDETGEIMDNLTLEAGDDAGKVKWMDISDKLKLYASHAQFIKSVAEKRDAHWSKDSETGCPG

SEQ ID NO: 128 - protein, 472 aa, Bos taurus
MKFLLFMFVGVVHLLPLASGKAIYGNGPSQRTFQEIKEEIAHYGDVAKSIINLTVYGKAQNRSYERLALLVDTVGPRLSGSKNLERAIEIMQQNLKGDGLENVHLEPVKIPHWERGEESAVMLEPRIHKMAILGLGSSIGTPPEGITAEVLVVTSFDELQRRGPDAEGKIVVYNQPYTNYSAAVQYRMEGAVEAAKVGALASLIRSVASFSIYSPHTGIQEYQKGVPKIPTACITVEDAEMMSRMASRGNRIVVQLKMGAKSYPDADSFNTVAEITGSKYPEQVVLVSGHLDSWDVGQGAMDDGGGAFISWEALSLIKDLGLRPKRTLRLVLWTAEEQGGVGSSQYYQLHKANSSNYSLVMESDLGTFLPSGLKFTGSDKARVIMEEVMSLLQPINITQVLKAGDGTDINFWIQDGVPGASLLDDLYKYFSFHHSHGDTMTVMDPKQMNVAAAVWAVVSYVVADLEEMLPRS

SEQ ID NO: 129 - protein, 529 aa, Bos taurus
MAGSTLRFSLLLAAAFAGRATALWPWPQYIQTSELRYTIFPQSFQFQYHLSSAAQVGCSVLDEAFQRYRDLLFGSVAFRFPHPIEKRHTSEKNSLVVLVVTPGCDQFPSLGSVENYTLTINDEQSLLLSETVWGALRGLETFSQLIWRSPEGTFYVNKTDIEDFPRFPHRGLLLDTSRHYLPLASILDTLDVMAYNKFNVFHWHLVDDSSFPYESFTFPELTKKGSYNPATHIYTAQDVKEVIEYARLRGIRVLAEFDTPGHTLSWGPGVPGLLTPCYSGSHPSGTFGPVNPALNNTYEFMSTFFLEISTVFPDFYLHLGGDEVDFTCWKSNPDIQAFMKKKGFGDDFKKLESFYIQTLLDIVSAYGKGYVVWQEVFDNKVKVRPDTIIQVWREEIPVKYVKELALVTRAGFRALLSAPWYLNHITYGPDWKEIYLVEPLAFEGSPEQKALVIGGEACMWGEYVDSTNLVPRLWPRAGAVAERLWSNKMVSNLDFAFKRLAHFRCELLRRGVQAQPLSVGYCDMEFEQT

SEQ ID NO: 130 - protein, 784 aa, Bos taurus
RRTLLVCSFILAAALGQMNFTGDQVLRILAKNEKQLSLLRDLEGLKPQKVDFWRGPARPSLPVDMRVPFSELKDIKAYLESHGLAYNIMIKDIQVLLDEEREAMAKSRRLERSTSSFSYSSYHTLEEIYTWIDTFVTEHSDVVSKLQIGHSFENRSILVLKFSTGGSRRPAIWIDSGIHSREWITHATSVWIAKKIVSEYSKDRIVTDVLNAMDIFLEIVSNPDGFAYTHSMNRLWRKNKSTRPGIFCIGVDLNRNWKSGFGGNGSNNNPCSETYHGPSPQSEPEVAAVVDFIMSHGNVKALISIHSYSQMLMYPYGHSLEPVSNQEELYHLAKDAVQALYEVHGIEYIYGSISTTLYVASGITVDWAYDSGIKYSFSFELRDTGRYGFLLPATQIIPTAQETYKVLRITAADEAEVQTVKELEDLEHLQLDFWRGPGQPGSPIDVRVPFPSLQAVKVFLEAHGIRYRIMIEDSRARSTNTFNYATYHTLDEIYDFMDLLVAEHPQLVSKLQIGRSYEGRPIYVLKFSTGGSNRPAIWIDLGIHSREWITQATGVWFAKKFTEDYGQDPSFTAILDSMDIFLEIVTNPDGFAFTHSQNRLWRKTRSVTSSSLCVGVDANRNWDAGFGKAGASSSPCSETYHGKYANSEVEVKSVVDFVKDHGNFKAFLSIHSYSQLLLYPYGYTTQSIPDKTELNQVAKSAVEALKSLYGTSYKYGSIITTIYQASGGSIDWSYNQGIKYSFTFELRDTGRYGFLLPASQIIPTAQETWLGVLTIMEHTLNNLY

SEQ ID NO: 131 - protein, 327 aa, Bos taurus
MAAMRRALLLLLYVSHASASCGIQKSNIAVTSEEGLVEEQEFPWVVSLQDSQYTHLTFGSILSEFWILSIASTFQNRKDAVAIIGIAKMDAKLIAHEEYPINTIIIHEDFDNKTMRNNIALLKTDTAMQFNSLVRPICFLGRKLNRPPVLMNCWVAGWNPTSATGNHMTMSILRKISVKDIDSCPLNIEQKTGCGNHLEMETNAVCLGDPGNPMMCQLKEMSLWVLRGILSQGGEKCPGLFLYIRVEDYSDWITSKTRKTSSPLSSFHHWENPSPLPSYSSRDLVTQRKQAGLSQLVWPRLSFQEQESSQWARGLPGTLYHMKRLRT

SEQ ID NO: 132 - protein, 885 aa, Bos taurus
PLLLTDVMLPRLLAVVRGPGSCRGWREGSPARGSASATVAPPVALPAQAQVVVCGGGIMGTSVAYHLSKMGWKDVVLLEQGRLAAGSTRFCAGILSTARHLAIEQKMADYSNKLYHQLEQETGIQTGYTRTGSIFLAQTQDRLISLKRINSRLNVIGIPCEIISPKKVAELHPLLNVHDLVGAMHVPEDAVVSSADVALALASAASQSGVQIYDRTSILHVMVKKGQVAGVETDKGQIQCQYFVNCAGQWAYELGLCSEEPVSIPLHACEHFYLLTRPWETPLPSSTPTVVDADGRIYIRNWQGGILSGGFEKNPKPIFTEGKNQLEIQNLQEDWDHFEPLLSSLLRRMPQLETLEIVKLVNCPETFTPDMRCIMGESPSVRGYFVLVGMNSAGLSFGGGAGKYLAEWMVYGYPSENVWELDLKRFGALQSSRTFLRHRVMEVMPLLYDLKVPRWDFQTGRQLRTSPLYDRLDAQGARWMEKHGFERPKYFIPPDKDLLALEQSKTFYKPDWFEIVESEVKCCKEAVCVIDMSSFTKFEITSTGDQALEILQYLFSNDLDVPVGHIVHTGMLNERGGYENDCSIARLSKRSFFMISPTDQQVHCWAWLKKYMPEDSNLILEDVTWKYTALNLIGPRAVDVLSELSYAPMTPDHFPSLFCKEMSVGYANGIRVMSMTHTGEPGFMLYIPIEYALHVYNEVMSVGQKYGIRNAGYYALRSLRIEKFFAFWGQDLNTLTTPLECGGESRVKLDKGVDFIGRDALLQQRQNGVYKRLTMFILDDHDTDLDLWPWWGEPIYRNGRYVGKTTSSAYGYTLERHVCLGFVHNFSEDTGEEQVVTADFINRGEYEIDIAGHRFQAKAKLYPVPSLLTHKRRKEDVELSDLHGK

SEQ ID NO: 133 - protein, 100 aa, Bos taurus
MKMSRLCLSVALLVLLGTLAASTPGCDTSNQAKAQRPDFCLEPPYTGPCKARIIRYFYNAKAGLCQTFVYGGCRAKRNNFKSAEDCMRTCGGAIGPWENL

SEQ ID NO: 134 - protein, 295 aa, Bos taurus; identical to SEQ ID NO: 55

SEQ ID NO: 135 - protein, 145 aa, Bos taurus
MKASVVLSLIGYLVVPSDTAVLGRCVVAKKLHEGGLSDFEGYSLENWVCLAYFESKFNPMAVYENSRSDFIGYGLFQIRNHDWCDHGRNRCHMSCSALLNPDLKKTIECAKTIVKGKRGMGAWPSWTLNCQHSDTLARWLDGCKL

SEQ ID NO: 136 - protein, 337 aa, Bos taurus
MEALPFSVEWYARWFPLTQTGIWKMCLCEFWLLLLGSGLKGHLLPDEEDVTFINEYVNLHNELRGNVLPRGANLRFMTWDVALSRTARAWGKKCVFERNTHLEELYMAHPKFNGIGENIWVGPENEFTASIAIRSWFEQRRKYNFEKNSCSGNCSNYLQLVWDRSYKVGCAVTPCSRIGRIKHAALFICNYAPGRGLSRRPYQRGLMCSQCDGYDRCTDFLCSNADRDQAIYYRYWYPRWEVPRPIVCNPLCLLILFLRMMCFSLSATIVLIIQSYFPNILVEEEMVFTPVETGPAKEKGKKEKQVEEEEEKEEEEEEEEEEGEEEKEEEGGEEEEE

SEQ ID NO: 137 - protein, 444 aa, Bos taurus; identical to SEQ ID NO: 58

SEQ ID NO: 138 - protein, 647 aa, Bos taurus
MAPKKPLCLPFLLLPLLTLLLPQADARSFVVDRDHNRFLLDGAPFRYVSGSLHYFRVPRVLWADRLLKMRMSGLNVVQFYVPWNYHEPEPGVYNFNGSRDLFAFLKEATLANLLVILRPGPYICAEWEMGGLPAWLLRKPKIHLRTSDPDFLAAVDSWFKVLLPRIYPWLYHNGGNIISIQVENEYGSYRACDVSYMRHLAGLFRALLGDRILLFTTDGPEGLKCGSLQGLYTTVDFGPADNMTKIFGLLRKYEPRGPLVNSEYYTGWLDYWGQNHSTRSIPAVTKGLEKMLKLGASVNMYMFHGGTNFGYWNGADEKGRFLPITTSYDYDAPISEAGDPTPKLFAIRNVISKFQEVPLGPLPPPSPKMTLGPLNLHLDGNLLDFLDFLCPQGPIHSVLPMTFEAVNQVHGYMLYRTYLPHTVSEPTQLWVPNNGVHDRAYVMVDGVFQGVLERNMKHNLFLMGKIGATLDVLLENMGRLSFGSNSSDFKGLLQPPILGQTILTQWLMFPLKVDNLVKGWFPIQPLKHSHPQTPSGPTFYSTTFPILNSGGDTFLFLPGWTKGQVWINGFNLGRYWTKRGPQQTLYVPRPLLFPRGAHNRITLLELENVPPQPQIQFLDRPILNSTKHRTYVYSLSSASEPMELSGH

SEQ ID NO: 139 - protein, 748 aa, Bos taurus
SLVLSLLCYICRSFTRSSALLTRTHINYGVRGDVAVIRINSPNSKVNTLSQELHSEFMEVMNEVWSSSQIRSAVLISTKPGCFIAGADLNMLNSCTTSQEVTQISQEAQKMFEKLEKSTKPVVAAINGSCLGGGLELAISCQYRIATKDKKTVLGSPEVLLGILPGAGATQRLPKMVGIPAAFDMMLTGRGIRADRAKKMGLVDQLVEPLGPGVKPPEERTIEYLEEVAITFAKGLADKKITPKRDKGLVEKLTSYALSIPFVRQQIYKKVEEKVRKQTKGLYPAPLKIIDVVKTGIEQGSNAGYLSESQTFGELAMTKESKALMGLYRGQTQCKKNKFGAPQKEVKQLAVLGAGLMGAGIAQVSVDKGLQTILKDTTLPALGRGQQQVFKGLNDKVKKKTLTSFERDAMFSNLIGQLDYRGFEKADMVIEAVFEDLNLKHRVLKEAEAVIPDHCVFASNTSALPIGEIATVSKRPEKVIGMHYFSPVDKMQLLEIITTEKTSKDTTASAVDVGLRQGKVIIVVKDGPGFYTTRCLTPMMSEVIRILQEGVGPKKLDSLTTSFGFPVGAATLVDEVGVDVAKHVAENLGKEFGERFAGGSLEMLQQMISKGFLGRKSGKGFYIYQEGVKNKNLNSDMDSILASLKMPSQPDVSSDEDIQYRLVTRFVNEAILCLQEGVLATPTEGDIGAVFGLGFPPCLGGPFHFVDLYGAQKIVDRLRKYEAAYGKQFTPCQLLVDHANNPNKKFYQ

SEQ ID NO: 140 - protein, 248 aa, Bos taurus
ATMKTFIFLALLGAAVAFPVDDDDKIVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN

SEQ ID NO: 141 - protein, 213 aa, Bos taurus
MALLLCLGLTAALARGCLHCHGNFSDKFSFYRHHVNLKSWWVGDIPVSGSLLSDWSQDTMKELHLAIPAEITREKLNQVANAVYQRMDQLYQGKMYFPGYFPNELRAIFREQVHLIQNAIIESRINCQRHCGIFQYETISCTNCTDSHVVCFGYNCESSAQWEKAVQGLLQYINKWHKMDTNTSLISPSFTCLEPPHLANLTLENASECLTQH

SEQ ID NO: 142 - protein, 191 aa, Bos taurus
MADLRILVSIILMTNAVLEKGGCTGPPGPPGHPGPPGIRGPPGIRGIPGLPGPPGTPGPSVKCPCHRQSAFTVKLSGQLPSPSKPVPFTEVLYNAQRDLQEDTGVFTCRVPGNYHFLFDVDLHHCKVTVQLMRDKSSVLEKHQVSTKEPRSLSGMLTLPLHVGEKVWLEAKVETEKPEQARVTIYFSGFLT

SEQ ID NO: 143 - protein, 4575 aa, Bos taurus
MGAPRPAVLLLLLLLFLDAANAQARDEVLENVSPSCPKDAVRFKHLRKYVYNYEAESSSGVPGTADSRSATKINCKVELEVPQLCNFILKTSDCTLKEVDGLNPEGKALLKKTKNSEEFAAAMSKYDLKLAVPEGKQVLLYPEKEEPKHILNIKRGIISALLLPPETEEAKQVLFLDTVYGNCSTDFTVKTKKGNVATEISIERNLEKCDHFQPISTGVSPLALIRGMTRPLSTLIGSSQSCRYTLDPRKKHVSEAICNEQHLFLPFSYKNKYGMMAQVTQTLKLENTPKINSRFFEEGTEAVGLAFESTRSTSPPNHAEAIVKTLQELQKLHVSEQNAQRANLFHKLVTELRGLSSEAVTSLLPKLIEVSSPITLQALIQCGQPQCYTHILQWLKNERANPLLIDAVTYLVALIPEPSAERLREVFNTARVQQSRLTFYALSHMINNYHKTNPTWTQDLLEIADYLSEQIGNDCTGNEDLTYLILRVIGNIGKTMEQLTPKLTSSVLKCIRSEQPSLVIQKAAIQALRKMELSDEVREVLLQTFLDDTSPGDKRLAAYLMLMGSPSQSDISKITQLLSGEQNEQVKNFVASHIANILNSEDLYVQDLKSLVEEALKKSQLPTIMDFKKFSRSYQFSKSISLPSLDLVSAKIEGNLIFDPNNYLPKESMLKTTLKVFGFASADLFEIGLEGKGFEPTLEALFGKEGFFPDSVNKALYWVNGRVPDRVSKVLVDHFGYTKDDKHEQDMVNGVMFNVEKLVKDLKSKDFPEARAYLRILGEELGFVKLHDLQLLGKLLLKGVRTLQGIPQMIKELIRKGSTSDLFLHYIFMDNAFELPTGLGLQLQVSSSGIITPGIKAGVKLEVADVRICSLSSRAGVLCNLKSILGRRIPDIFRSSSDSKVLIWPLEKGVEWRQEGARRAGKGCGVCVHSGKRDLSMFSAGGENNTLHLVSTTKTEVIPPLIENRQSWSTCKPFFIGLNYCITGAYSNASSTDSSSYYPLTGDTRFELELKPTGEVEQYSAKAFYELRRDGEALVDMLKLVTQTEGVKQTEATLLFRYNRQSKTLSSELHIPDFDIDLGTVLKVNYEHSKEKKSYKLTLDFQNKKITEVTLTGHISYDRKEEGKIKGVISIPRLQAEARSEILSLWSPSKLLLQMDSSATAYGSTISKKVAWRYDGDKIEFEWNTGTNVDTKKATSNFPVDLSAFPKSLHTYANILLDRRVPQTDLTFRHMGSKLVAAASTWLQETSRSLPYAQNIQDQLSGLQELCLQKMELPNFHIPDNLFLKRLTVGIKYTLNKNSMEIEIPLPFGGKSSDLKMFKTIQTPALNFQPLGFHLPSQEFQIPTFTIPNLYPLRLPLLGVLDLSTSIYSNLYNWSASYTGGNTSTDHWSLQAQYHVKADSVVNLLSYHVQGSGKTAYDHRNTLTFSGDGSLHHKFLDSNVKFSLVEKRGNSPASEGLLTFDASSAWGPQISASVHLDSKKKEHLYVKEVKIDGQFRASAFYAKGAYDLSYQRDPATGQLTGESNLRFNSSYVQGTNQITGRYDDGTVSITSTSDLQDGLVKNTASLKYENYELTLKSDTNGKYEDFATSNKVDLTFSKQHALLRSEYQADYKALRFFTLLSGSLNSHGLELNADILGTDKINSGAHKATLRIARDGMSTSATTSLKYNPLVLENELNAAVSLSGASLKLTSNGRFREHHAKFSLDGKAAPTEVSLGSAYQAMILGVDSKNIFHFKINQEGLKLSNDIMGSYDEMKLDYTNNLNIVGFSLDFSSKLDNIYSSDKFYKQNFNLQLEPYSLVTTLNSDLKFSALDMTNNGKLRLEPLKLNVGGTIKGAYQNDEIKHIYTLSYADLSASYKADTVAKVQGTEFSHRLNTNIAGLASTVDIRTNYNSDSLHFSNAFHSAVAPFTMTIDTHTNGNGELVLWGQHTGQLYSKFLLKAEPLAFTFSHDYRGSTSHRLMSKRSITTALDHKVSALLTLAEQTGDWKLRTQLNQNEYSQDFSAYNTKDKAGVELSGRALADLTVLDFPIAVPLVLNEPINVIDTFEMRDSVGQPQEFTLVASVKYDKNQNAHTIHLPFFKFLPKYFEKTRVVVIGALETVQKELKRTHIDKHMRKYVEALDRLSQQVSDYLSTFNWERQVLSAKEKLTAFTENDLQIVLDNATINLNEKLSQLQTYVIQFDQYFEDNYDLHDFKTTIAKIIDQIIEKLKILDEHYHIRVNLVKKIRDLYFFIENFDFNKTESSTASWIQNMDTKYQIRIQIQEKLQQLKVQIQTVDIHHLAEMFKQQVEAVDVRVLLDQLRTTIPFQRIKEIIEHVKYRVTSLSEGFKVTEEINAFRVMVHEFIKMYKIDQHIQALMDKSVQLAQQYKLQETVQKLSSVLQQVKMAEIVEKLIGLTDNALKQLEALSFNQFLEEVNRFLDMLVKKLRSFDYHQFVDETNNKIHEVTQRINDEIQALEFPQKAKALRLLVEDARIVVSTYLENLKATKITLVLDWLQEALSSTSVTYMKAKFQETLEDFRDRVYQMDVQQELQRYLFLVGQVYNTLVTYISDWWALAAKNLTDFAEQYSIQDWAENLKTLVEQGFTVPEIQTTFGTIPAFKIGLRALQEATYQTPDFIVPLTDLRIPSFQINFKTLKDVKIPSSFSTPEFTILNTLHIPSFTIDFVEIKIKIVRTIDQMLNSELQWPVPEVYLGDLSGVDTILAGITVPDFHLPEITIPEFVIPNLNLTDFQVSDLHIPEFQLPHISHTIEVPAFGKLYSILKIQSPFFTLDANADIQNATALANQAEMEASITAKGASRLEVLNFDFQAKAQLSDPTLNPLVLKEFVRFSSKYLKTEHESEVLWVGNAIEGKSNTVAGIHTEKNTLKLSSSVLVNISNQLTLDSNTKYFHKLNIPQLDFSSQADLHSDLKTLLEAGHIAWASSGRASWKLACHEFSDEGTHESQSSFTVEESITSFRLSNKINSKHLRVNQKILYESRFLNFSKFEIQSQAECQHVGRSVLTAKGLALLQEGKAEITGNHDAHLNGKVVGTLRNSLFFSAQPYEMTASTNNEGNLRVSFPLKLTGKIDFLNNYALFLSPSTQQASWQASGRFNQYKYHQNFSAGNNEKSIEAHIGINGEANLDFLNIPLKIPEMTLPYTKLTTPQIKEISLWKKTGLKDFLKTTKQSFDLSVRAQYKKNKDKHSIPVPLDAFYALIHRNINSFSRLFETVRNQALDFFIKSYNEAKTTFDKYKVEKSLNQQPKIYKIPGYTIPVANIEVSPFTVKMLTFEYMIPKEISTPSITVLGSSISVPSYTLVLPFLELPALPVPKELSELSLPEFKVLSTINKILIPALGNITYDFSFKSSVITLNTNVGLYNQSDIVAHFLTSSSSVMDTLQYKLEGTSSLTRKRGLKLATALSLSNRFVEGNHDSTVSFTKKNVDASVTTTARVQIPILKMSFKQELHGNTKSKPTVSSAIELKYNFNSPKLLSAATGAVAHKLTLESLTSYFSIESSTKGGVEGSFGSWGYSGSLASEANTYLNSKGTRSSVTLQGASKADGLWDLKVKENFAGEATLQRIYIIWEQNMKNHLELGGLILTSGEHTSKATLELSLWKISALVQVRAHQPNSLLKINFLGQEVSLNVNTENQKISWNSEVQVPSGSLQNNIQLSNDQEVARFDMAVSLEGYLRFLKDTVLPVYDKSLWDLLKLNVTTSLNRKQYLRAATALIYTKNPNGYLVSIPMQELDDKFIIPGLKRNNQNSIHVTPTFQVPFTDLQIPSYTLDLSEIKIYKNLSTLPLTFNVSTLSQVKFPRVDVLIQYSKPEASSVPFFEITVPASQLTVSQFTLPKSISVGSTVLDLNTVASNIADSELPTITVPEQTLEIPSMKFSVPAGILVPSFGTLTARFEVASPLYNTTWSAGLKNKGDHVETFLDSTCSSTIQFLEYDLNVVGTHKIEGGMLVYKTNASFAHRDFSAEYEEDGKYQGLGYWNGKVDLNITSPTLTDVCLHYQEDENHFSSSVAFPAIGTVVIDVKNISDTVLGWNLYYRPQASPDKILNIFKIELRVLELDDEVQIKANWEEGAASELLSSLKDSVPRATGALYDYVNKYHQEYMGLDLKDATLKLRRGLQDSADQVYQGAMRQIDEVDMELQRMARGTTETYQQWKDKAQSVYQELLAQEDRSGFQRLQKVSDSLIGVTQGYSVTVKHATDLFIDLLNVTRFQLPGRARNYTADELCTVVIQEIAKRLSQVHSNIHKGLHILFSYLLYLMEESELYKELNNKFSFTSMCNQLIDMALECMKTFLFFSQEIQKALSYLQSTMMTEMLSDLQRSLQDIFQEIESELKRFKEKKLTDFINQTLQNINTISDIYISFVFRFLKENLNYNFVEFNKLIQNKLQVASQELQQLLQYVQALHQEYFDPTTVGWTVKYYEFEEKVINLIKNLVDVLKDFHSKYIVGATDFASQLSSQIEPLIQKSLQEYLSILANAEGRGKEKITELSTSAQEVIKSWAVAVKEIISDYHQQFTYKLQEFSDQLSDYCEKFISESKRLIDLSIQNYHMFLNYIMELLKELQSATINDMSPYIKVAPGEFTITF

SEQ ID NO: 144 - protein, 152 aa, Bos taurus
MRAWVLLSAVLWYLTGVGWQRNSERTEKGFIYGKPGHPVKVYVKLHHNSPVLVCMDFKRAEKETVDPTYLWVGPNEKQLSGSNRINITNTGKLTVKDFLEPLSGLYTCSFSYKTIKAETQEEKIVKQRYDFMIFGKKSNRRIFPEPSIYFQP

SEQ ID NO: 145 - protein, 243 aa, Bos taurus
MKCCVFFMLMSTAGVILAADSNTEKRNEVDFTIPYMVYLQSFPEPCVGTLIHPQWVLTAAHCPSPVKIRLGIYQPNIKNKHEQTQSYSMTVPYPDFNSEYLSNDLMMIKLSEAAKVTTQVGTIAIAMEPLSLNESCFIPTWTWSKYKNLSDPDILTWINQYTRPYDECQNMQDERMAANVLCVGQPLKTLSDAKEVSAAPAICGGRVHGILSWAKGSVTLGNEGFFTEISSYSRWIMSIIESY

SEQ ID NO: 146 - protein, 168 aa, Bos taurus
MQSLKWCLFLPLCLSCGYAFMFSSLREKPKETQGKVPCGGHFRIRQNLPEHTQSWLGSKWLWLTFIVVLYMILKFRGDGEKNKVQTPPTLRGCTFRPPGRKNASPNKNYAFSTLTQLEMDLVEFVSRVRNLKVSMTTSGHFRLQNLGNADPQHKVTIYEVWGEEDSVD

SEQ ID NO: 147 - protein, 307 aa, Bos taurus
MVPAAGALLWALLLSLGPRAAGAEGLTSTAMPRDSFRFGGPMTRSYRTTSRTAPGVVSPRMRVTMEDEDDVLAAADRLAGPAAAELLASTVATGSRSRLSQEEDGSLEEGVVIYARKNDTESETDSTTRITPGRPSPYLTANDQDSDIKMSSSLPHSSWKANVDSQPSDSTTNPWSSAGSTLNQWPPPSPTAMPAPEDLRLVLLPWGPWHCHCKSGTMSRTRAGRLHGLSGRLRVGALSQLRTEHRPCTYHLCPCNREREECPLDADKEKSEVMATSTTRGGVSISTSPPPRIPVPTGYIFSTEKQN

SEQ ID NO: 148 - protein, 128 aa, Bos taurus
VFRLLLMSMFMVLFVNERDRVLGANKWFQLCRRCEFHNGYSCNHVIGSCWKFKIANLKRACVTDNFYYYDRVADRYNYRYSVLSCRTCEEGSFMLFHDLYRETFCCTEDNCNDPEKNMDISKIAMSYP

SEQ ID NO: 149 - protein, 273 aa, Bos taurus
MKNFLLLVSLCFLGSARAASAQSDEFLGSMDHQASVQQLSSDYFSLANPLDEVLYETSSIDNTLSERGSSETAAAQHASGEPLAVEHASGPTPVDHATAVEHAPGEAAAGEHTLNEHTPGEQLSVEQPIGEQPLGEQSVGEQAVGEQPTNEQVASDKPAGEQSAVEPISAEQVSAESSAEQPSELPPSSTFSGPPLSCHTCSYMNDQGKCLRGEGVCSTQNSQQCMLKKIFEGGKLQFMVQGCENMCPSMNLFSHGTRMQIICCRNQSFCNKI

SEQ ID NO: 150 - protein, 286 aa, Bos taurus
RMPVLDFPSCPRLPSLLLALLLKLTGTSVEDFQVNQPESSVSAKVGDVITLGCNIPAPSPVGPVLWFKDARLERKLIYSFNGRRFPRVSPVVNPMANQTDYSIRIRDVSPKDAGMYYCVKLTIGHPDMAYIAGPGTYVSVSGVTHEMFKVQQAEISQTVSPGETLTLSCSIPDSFPNGPVLWFKGTGPNRELIYNFQRGLFPRVNQIGNMAKAGNKDFSIRISEISLKDAGTYFCVKFKEGNPDIEYQSGRGTKVTVTGTRNNFVPDTPGRHFLATRGTNLNKNLL

SEQ ID NO: 151 - protein, 276 aa, Bos taurus
MRIAVICFCLLGIASALPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDCGLRTEVKIKFRRSNVQSPDATEEDFTSHIESEEMHDAPKKTSQLTDHSKETNSSELSKELTPKAKDKNKHSNLIESQENSKLSQEFHSLEDKLDLDHKSEEDKHLKIRISHELDSASSEVN

SEQ ID NO: 152 - protein, 255 aa, Bos taurus
KSGLSGLAALRGLLCGARALGPSLLARSCSGGPSWTRERTLVAVKPDGVQRRLVGDVIRRFERRGFKLVGMKMLQAPERILAEHYHDLQRKPFYPALISYMSSGPVVAMVWEGPNVVCTSRAMIGHTNSAKAAPGTIRETSAPTSAGMSSMPVTPWRGPRERSSCGSRAVSWWTGQMKATSAASTQPEHTGTRGHPGIHSGPATSVDKNLSSHPRSAHLSQTTSLSTFCPTPAPEWLSHKLYVPFSKPATGPSPF

SEQ ID NO: 153 - protein, 152 aa, Bos taurus
MAAVLTFLRFLGRGGAVTRGLPGGARCFGVRTSPTGEKVTHTGQVYDDGDYRKVRFVGRQKEVNENFAIDLIAEQPVSQVGSRVISCDGGGGALGHPRVYINLVMCCPAPSPSPGSCPVLSPLAAHARLGPAHESSARAEQRAGTHRASRTG

SEQ ID NO: 154 - protein, 473 aa, Bos taurus
MLRWLRVFVLPAATCHTDEEDYLHYKNLFQDLDHNGDGVVDILELQEGLKNWNPSFAREKDIYKTVDTNADSGLNFEDFMRYVKDHERKMTLAFNSLDKNNDGIIETSEIIAVLKSLGINISETQAKKIIQSIDRDGTMTVDWDEWKNYFLLHPAKNIDEIAHFWKRSTMIDIGESIAIPDDITEQEKRSGNWWKRLVAGGIAGGVARTCTAPFDRLKVMMQIHSLQSGKMRLLDGFKQMVKEGGILSLWRGNGVNVLKIAPETALKVGTYEQYKKWLSSDGAKIGIIERFISGSLAGATAQTCIYPMEVIKTRLAVGKTGQYSGIIDCGKQLLKQEGARAFFKGYIPNLLGIIPYAGIDLCVYEHLKNHWLEHHARGSLDPGIAILLGCSTLSNACGQMASFPLNLIRTRMQAQALEEKGTTSMIQLIQDIYNKEGKRGFFRGVTPNIIKVLPSVCISCVTFEKVKGHVGFT

SEQ ID NO: 155 - protein, 247 aa, Bos taurus
MKCCVFFMLMSTAAPKSGVILAADSNTEKRNEVDFTIPYMVYLQSFPEPCVGTLIHPQWVLTAAHCPSPVKIRLGIYQPNIKNKHEQTQSYSMTVPYPDFNSEYLSNDLMMIKLSEAAKVTTQVGTIAIAMEPLSLNESCFIPTWTWSKYKNLSDPDILTWINQYTRPYDECQNMQDERMAANVLCVGQPLKTLSDAKEVSAAPAICGGRVHGILSWAKGSVTLGNEGFFTEISSYSRWIMSIIESY

SEQ ID NO: 156 - protein, 183 aa, Bos taurus
MAPLVGLFLIWAGASVFQQLHPVNGGDTPLPSISSAPAFLLDELPTETYDLPPEIYTTTFLPRTIYPQEEMPYDDKPFPSLLSKANDLNAVFEGPACAFPFTYKGKKYYMCTRKNSVLLWCSLDTEYQGNWKFCTERDEPECVFPFIYRKKSYESCTRVHSFFWRRWCSLTSNYDRDKAWKYC

SEQ ID NO: 157 - protein, 553 aa, Bos taurus; identical to SEQ ID NO: 4

SEQ ID NO: 158 - protein, 499 aa, Bos taurus
MGRCSLLLLLLLIAFLTPGAANPVSPSLRAPSSLPWSTSFRSRPTITLKYSIRYIQQKVDHFGFNIDRTFKQRYLIADNYWKEDGGSILFYTGNEGDIIWFCNNTGFMWDIAEEMKAMLVFAEHRYYGESLPFGADSFSDSRHLNFLTTEQALADFAKLIRYLKRTIPGARNQPVIALGGSYGGMLAAWFRMKYPHLVVGALASSAPIWQFNDLVPCDIFMKIVTTDFSQSGPNCSESIRRSWDAINRLAKKGPGLRWLSEALHLCTPLTKSQDVQRLKDWISETWVNVAMVDYPYESNFLQPLPAWPVKVVCQYFKYSNVPDTVMVQNIFQALNVYYNYSGQAKCLNVSETATSSLGVLGWSYQACTEMVMPTCSDGVDDMFEPHSWNMKEYSDDCFKQWGVRPRPSWIPTMYGGKNISSHTNIIFSNGELDPWSGGGVTKDITDTLLAIVIPNGAHHLDLRASNALDPVSVQLTRSLEVKYMKQWISDFYVRLRKMN

SEQ ID NO: 159 - protein, 147 aa, Bos taurus
MASFRLFLLCLAGLVFVSEAGSVGAGEPKCPLMVKVLDAVRGSPAANVGVKVFKKAADETWEPFASGKTSESGELHGLTTEDKFVEGLYKVELDTKSYWKSLGISPFHEFAEVVFTANDSGPRHYTIAALLSPYSYSTTALVSSPKA

SEQ ID NO: 160 - protein, 348 aa, Bos taurus
AAMAWKPGLLLPLLLLLLGPAPARSFGQLPLVLNTWPFRNATVAAWKTLAAGGSALDAVESGCATCEQQQCDGSVGFGGSPDESGETTLDAMIMDGTTMNVGAVGDLRRIKNAIGVARKVLEHTTHTLLAGEAATKFAESMGFINEDLSTNVSQALHSDWRARNCQPNYWKNVIPDSSKYCGPYKPPTVLKRDGITYEDTAQSYGHDTIGMVVIHKTGNIAAGTSTNGIKFKIPGRIGDSPIPGSGAYADDMVGAAAATGDGDILMRFVPSYQAVEYMRRGENPTTACEKVISRIQKYFPKFFGAVICANVTGSYGAACNKLSTFTQFPFMVYNPLKSAPTEEKVDCI

SEQ ID NO: 161 - protein, 163 aa, Bos taurus
MAAAVVLAAGLCVARRAVAVAGPRGVQVRGAAGVTDGDEVAKAQQAAPGGAAPTIFSRILDRSLPADILYEDQQCLAFRDVAPQAPVHFLVIPKKPIPRISQAEEEDQQLLGHLLLVAKETAKAEGLGDGYRLVINDGKLGAQSVYHLHIHVLGGRQLQWPPG

SEQ ID NO: 162 - protein, 223 aa, Bos taurus
MNGWPSYLLGWMAFLLYSYGTTGNKKDSCIFPFTYKGSTYFSCTRANSLSPWCSTRAIYDGRWKHCLTEDYPRCVFPFIYRGKPHNGCITDGSLFGRQWCSVTSSFDEKQQWKYCETNEYGGNSFSKPCIFPATYRNHVVSECLEDENNKLWCPTTENMDKDGKWSLCADTRISSLVPGFPCHFPFNYKNKNYFNCTNKGSKENLLWCATSYNYDRDHTWVYC

SEQ ID NO: 163 - protein, 121 aa, Bos taurus; identical to SEQ ID NO: 2

(19) United States
(12) Patent Application Publication
(10) Pub. No.: US 2014/0315185 A1
(43) Pub. Date: Oct. 23, 2014

(54) METHODS AND COMPOSITIONS FOR IMPROVING SPERM FUNCTIONALITY

(71) Applicants: Bridget Lee Dicker, Orakei (NZ); Jiwon Hong, Auckland Central (NZ); Keith Hudson, Titirangi (NZ); Shakeela Nathalia Jayasinghe, Manukan City (NZ)

(72) Inventors: Bridget Lee Dicker, Orakei (NZ); Jiwon Hong, Auckland Central (NZ); Keith Hudson, Titirangi (NZ); Shakeela Nathalia Jayasinghe, Manukan City (NZ)

(73) Assignee: ANDROGENIX LTD., Auckland (NZ)

(21) Appl. No.: 14/237,549

(22) PCT Filed: Aug. 8, 2012
(86) PCT No.: PCT/NZ2012/000140

§ 371 Date: May 1, 2014

Related U.S. Application Data
(60) Provisional application No. 61/522,609, filed on Aug. 11, 2011.

(51) Int. Cl. C12N 5/076; A01N 1/02; C12N 9/96

(52) U.S. Cl. CPC C12N 5/061; C12N 9/96; A01N 1/0221
USPC 435/2; 530/331; 530/350; 530/395; 435/188; 435/375

(57) ABSTRACT

Methods for improving the functionality and/or fertility of sperm, for example, by enhancing motility and extending the lifespan of sperm in the FRT, by adding functional molecules of interest attached to a membrane anchoring agent-PEG conjugate to the surface of the sperm are provided. Such methods may be used in AI to reduce the number of sperm needed for insemination and to improve conception rates.

### FIELD OF THE INVENTION

This application relates to methods for enhancing the functionality of sperm. More particularly, this application relates to methods for reducing the number of sperm required in livestock artificial insemination (AI) in particular for application with flow cytometry semen sexing. The methods may also be employed to increase the fertility of sperm in some human male individuals with sub-optimal fertility.

### BACKGROUND

The ability to identify and select male and female sperm has great value in the livestock industries, where there is an established market in AI of over US$2 billion per annum in the Organization for Economic Cooperation and Development (OECD) countries. This is particularly true in the dairy industry where over 80% of dairy farmers in key OECD markets impregnate their cows through AI. Sexed semen provides the opportunity to increase farmer productivity and income. For example, the availability of sexed semen has significant impact in reducing and/or eliminating the minimal returns of male dairy calves as compared to female calves.

Currently, the only commercial technique for semen sexing uses flow cytometry to sort sperm on the basis of DNA content. Bovine sperm bearing the Y chromosome have approximately 4% less DNA than sperm bearing the X chromosome. This difference, in combination with a fluorescent DNA binding dye (for example Hoechst 33342) and flow cytometry, permits purification of X chromosome bearing sperm to greater than 90% (Johnson et al., 1989). However, the ability to sort bovine sperm is currently limited to a rate of 8000 s⁻¹ which, when each straw, or dose, contains 2×10⁶ sperm, translates to 14 straws/hour (Sharpe and Evans, 2009). As a result, sexed semen straws generally incorporate ten-fold less sperm than unsexed straws. In addition, the sorting process itself has a negative effect on the fertility of the sperm. The reduction in the number of sperm per straw, together with reduction in sperm fertility due to the sorting process, causes a significant reduction of 14% in the conception rate for sexed sperm compared to unsexed sperm (Frijters et al., 2009). The sexed semen also has a significant price premium over unsexed sperm due to the high cost of sorting even the sub-optimal number of sperm used in the sexed semen straws. A valuable addition to the semen sexing technology would be a method to enhance the fertility of sperm so that a dose of considerably less than the approximately 2×10⁶ sperm per straw currently used for sexed semen would achieve the same conception rate as the normal, unsexed, straws.

Such methods would also have application in swine AI where much higher doses of sperm are used in the standard AI methodology than with bovine, namely approximately 2500×10⁶ sperm per straw. Recent work suggests that more sophisticated techniques involving deep intrauterine insemination can lower this requirement to 50-70 million (Vazquez et al., 2005; Vazquez et al., 2008). However, this reduced dose is still beyond the present commercial capability of flow cytometry sorted sperm.

### The Sperm Journey in the Female Reproductive Tract

Sperm are highly specialized cells that deliver the haploid male genome to the haploid female genome contained in the oocyte. Despite this seemingly simple mission, the path to achieving this goal is highly complex. Extraordinarily large numbers of sperm are inseminated in a natural mating, for example approximately four billion sperm/oocyte in the cow. The inseminated sperm spend a variable period of time, ranging from hours to days in the different regions of the female reproductive tract (FRT). The environments that sperm encounter from ejaculation to fertilization of the oocyte also vary considerably. These environments range from the complex molecular mix added to sperm at ejaculation by the male to the various female secretions and different cell surfaces of the female epithelia (Drobnis and Overstreet, 1992).

Once sperm are deposited in the FRT, a combination of active sperm migration and female uterine muscle contraction propels the sperm to the oocyte. During the journey through the FRT, sperm can be retained in specialized regions, most notably the cervix and oviduct (Drobnis and Overstreet, 1992). This retention may increase the probability that at least some sperm will be present in the oviduct at the same time as ovulation occurs. However, for the cervix it is more likely that the restriction of entry and trapping acts as a negative selection imposed against sperm by the female. In fact, one of the major innovations that launched the modern bovine AI industry was the finding that considerably less sperm were required when sperm were passed through the cervix and deposited into the body of the uterus (Foote, 2002). The final phase of the sperm journey in the oviduct involves release of sperm from the isthmus region (controlled by the female) and travel to the ampulla for fertilization of the oocyte. At this time near unitary numbers of sperm are present (Drobnis and Overstreet, 1992). Fertilization itself is again a complex phenomena involving penetration of the cumulus oophorus and subsequently the zona pellucida (Katz et al., 1989). Although the sperm journey through the FRT is broadly similar between mammalian species, various aspects do differ.

Sperm also undergo a maturational change while resident in the FRT, known as capacitation. When sperm are ejaculated, they are not capable of fertilizing the oocyte. However, during passage through the FRT sperm gain the capacity to fertilize. Changes to sperm during passage through the FRT include alterations in membrane properties, enzyme activities and motility (Salicioni et al., 2007). One such important change is the loss of cholesterol from the outer sperm surface membrane (Flesch et al., 2001; Osheroff et al., 1999; Visconti et al., 1999). Ultimately these changes enable sperm to respond to stimuli that induce the acrosome reaction and penetration of the egg. One of the important changes that occur during capacitation is alterations in the surface properties of sperm. A specialized protein-carbohydrate coating stabilizes the surface membrane (Schroter et al., 1999), regulates capacitation (Topfer-Petersen et al., 1998), facilitates transport through the FRT (Toliner et al., 2008b), and enables attachment at the oviduct (Tollner et al., 2008a). In different species, essentially the same functions are carried out by the surface coatings, however the specific molecular components vary (Calvete and Sanz, 2007; Tollner et al., 2008a; Topfer-Petersen et al., 1998).

### The Attrition of Sperm in the Female Reproductive Tract

In a natural bovine mating, approximately 4 billion sperm are inseminated yet less than 10,000 get to the oviduct and less than 10 get through to the oocyte (Mitchell et al., 1985). Why there are such large losses is poorly understood. Following coitis, greater than 80% of sperm are lost through vaginal discharge (Mitchell et al., 1985). The remaining sperm form a gradient in concentration from the cervix to the oviduct (Hawk, 1983; Hunter, 2003; Mitchell et al., 1985). In bovine, approximately 10,000 sperm arrive at the oviduct 6-8 hours after insemination (Mitchell et al., 1985). By 12 to 24 h after insemination, sperm have either been lost through back flushing, eliminated by phagocytosis or reached the oviduct (Hawk, 1983). In pigs, there is strong evidence for phagocytosis of sperm by polymorphonuclear neutrophils, with a massive infiltration of neutrophils occurring in the uterine lumen shortly after insemination (Matthijs et al., 2003). Recently, similar evidence that neutrophils infiltrate the uterine lumen after insemination in cows has been published (Alghamdi et al., 2009).

### How Sperm are Damaged During Passage Through the Female Reproductive Tract

Experimental evidence suggests that both motile and damaged (immotile and/or dead) sperm are lost by discharge (Lightfoot and Restall, 1971; Oren-Benaroya et al., 2007). In contrast, in vitro evidence indicates that live sperm are preferentially phagocytosed by neutrophils (Woelders and Matthijs, 2001). Several phenomena contribute to sperm damage from the FRT, although the mechanism and significance are poorly understood. Such phenomena include:
Adhesive properties of female epithelia capturing sperm and/or damaging the sperm surface, particularly mucus laden surfaces such as the cervix. This occurs by both biochemical and physical shearing (Katz et al., 1989; Mullins and Saacke, 1989).
Female secretions modulating and/or damaging the sperm surface or functionality such as flagella activity, capacitation and acrosome status. Such secretions include antibodies, complement components, molecular species affecting energy, osmotic and oxidation homeostasis, signaling molecules particularly for capacitation, and also catabolic entities. Microorganisms that are present in the FRT may also secrete agents that affect sperm.

Sperm also cause damage to themselves through generation of reactive oxygen species (ROS) mainly as a by-product of mitochondrial function (de Lamirande and Gagnon, 1995; Koppers et al., 2008; Vernet et al., 2001). ROS cause loss of sperm motility and lipid peroxidation. The latter damage leads to alteration of the membrane properties such as flexibility and fluidity, and can also lead to lack of membrane integrity and/or decreased chromatin quality (Storey, 1997). Sperm are particularly sensitive to ROS-induced damage because of their membrane composition and their limited antioxidant defenses. In particular, the high proportion of polyunsaturated fatty acids (PUFA) in the surface membrane makes this membrane highly susceptible to oxidation (Jones et al., 1979). The nature of the sperm cell, with limited cytoplasmic fluid, also constrains the availability of intracellular antioxidants (Koppers et al., 2008, & ref within). In human sperm at least, there exists a strong relationship between ROS production and antioxidant protection for determining the lifespan of sperm in the absence of external damaging agents (Alvarez and Storey, 1985; Storey, 1997, 2008).

### Sperm Motility, but not Viability, is Sensitive to Specific ROS

Sperm from human (Bell et al., 1992), bovine (Bilodeau et al., 2002), equine (Baumber et al., 2000) and porcine (Awda et al., 2009) all show loss of motility when exposed to H₂O₂ concentrations in the mico molar range, however where examined this loss of motility has not been associated with loss of viability (Awda et al., 2009; Bell et al., 1992). In addition this ROS-induced loss of motility is specific to H₂O₂ and not other ROS like .O₂⁻ (Awda et al., 2009; Baumber et al., 2000; Bilodeau et al., 2002). The mechanism for H₂O₂-induced loss of motility is currently unknown, however it may be related to the observation that, unlike other ROS, H₂O₂ is able to pass through the cell membrane (Bienert et al., 2006). This membrane passage by H₂O₂ has so far been shown to be facilitated by aquaporin membrane proteins. Sperm express surface membrane aquaporin pumps, which are thought to be associated with sperm volume regulation by being able to pump H₂O through the cell membrane (Chen and Duan, 2011; Chen et al., 2011; Yeung, 2010).

Sensitivity of sperm motility to H₂O₂ may also occur in the FRT. It has been shown that dead sperm in combination with aromatic amino acids produce H₂O₂ (Shannon and Curson, 1972; Tosic and Walton, 1950) and dead sperm are abundant in the FRT. H₂O₂ may also result from bacterial organisms present in the FRT. In particular, Lactobacillus acidophilus is known to produce H₂O₂ and is frequently present in both the human (Klebanoff and Smith, 1970) and the cattle vagina (Otero and Nader-Macias, 2006).

In all species where sperm suffer H₂O₂-induced loss of motility, antioxidants such as catalase and glutathione are able to rescue the loss of motility if added in sufficient concentration and simultaneously with the H₂O₂ (Baumber et al., 2000; Bilodeau et al., 2002). In contrast, antioxidants reactive against .O₂⁻ such as superoxide dismutase (SOD) could not rescue motility (Baumber et al., 2000; Bilodeau et al., 2002; Lapointe and Sirard, 1998).

Surface Properties of Sperm May have a Significant Influence on Fertility

The specialized protein-carbohydrate coating that facilitates transport through the FRT (Tollner et al., 2008b), may influence fertility by protecting sperm from mucus capture in the FRT or assisting the motion of sperm through mucus. An example of a protein that coats the surface of sperm and facilitates travel through the FRT is β-defensin 126 in macaque monkeys. This highly sialylated glycoprotein coats macaque sperm and is a major component of the sperm glycocalyx (Yudin et al., 2003; Yudin et al., 2005). Importantly, this glycoprotein facilitates movement of sperm through cervical mucus (Tollner et al., 2008b) as does the human β-defensin 126 on human sperm through the cervical mucus surrogate, hyaluronic acid (Tollner et al., 2011). Men that are homozygous for a deletion mutation of β-defensin 126 exhibit impaired sperm function and subfertility (Tollner et al., 2011). Macaque β-defensin 126 has extensive O-linked-glycosylation in the carboxy-terminal portion of the protein and a significant amount of sialic acid on the carbohydrate-terminal residues (Yudin et al., 2005). The negatively charged sialic acid residues from β-defensin 126 contribute the majority of the charge on the macaque sperm surface (Yudin et al., 2005) and presumably also on human sperm (Tollner et al., 2011). These sperm surface charges may well be responsible for penetration through the negatively charged cervical mucus or substitutes with similar properties of charge and viscosity like hyaluronic acid (Aitken et al., 1992; Tang et al., 1999). If surface charge is important for sperm movement through mucus, changing either the actual surface charge or the distribution of charge may affect sperm motion in uterine mucus and fertility.

In summary, the FRT is hostile to sperm, in particular selecting for motile non-damaged sperm but also removing the vast majority of sperm. While in the FRT, sperm have to deal with a wide variety of physiological environments, mature particularly at the cell surface and respond appropriately to signals at the right time and place. Thus despite the sperm's simple mission and relatively simple construction, successful sperm have the characteristics of at least reaching the upper uterine horn, remaining undamaged (mainly a surface phenomena), not being phagocytosed, remaining motile (a function of mitochondria, glycolytic enzymes and flagella components), avoiding capture by mucus and being able to respond to signals appropriately (a surface phenomena but also involving signal transduction and effector pathways). Treatments to sperm that enhance the ability of sperm to remain undamaged, motile, not phagocytosed and functionally competent could therefore reduce the number of sperm required for insemination.

### Pegylation

Polyethylene glycol (PEG) has the general formula: H(OCH₂CH₂)nOH with typical molecular weights of 500-20,000 daltons. It is non-immunogenic and soluble in aqueous solutions. The polymer is nontoxic and generally does not harm active proteins or cells.

Pegylation of proteins has been shown to improve solubility and vascular longevity, and decrease the immunogenicity of xenogeneic proteins while retaining normal protein function (Abuchowski et al., 1977a; Abuchowski et al., 1977b; Jackson et al., 1987; Senior et al., 1991; Zalipsky et al., 1994). Pegylation has also been used directly on cells to provide immune camouflage, initially for transfusion of red blood cells (Chen and Scott, 2001; Scott et al., 1997) and subsequently for other tissues such as pancreatic beta islet cells (Chen and Scott, 2001; Teramura and Iwata, 2009). For both red blood cells and pancreatic beta islet cells, the respective cell functions were preserved despite the pegylation.

### SUMMARY OF INVENTION

The present disclosure provides methods and conjugates for improving the functionality of cells, such as sperm. More specifically, the present disclosure provides conjugates that can be employed to attach functional molecules of interest, such as proteins or carbohydrates, to cells. The disclosed methods and compositions are effective in improving the functionality and/or fertility of sperm in the FRT, for example by preventing loss of motility, protecting against phagocytosis by neutrophils or other immune attack, facilitating sperm movement through the FRT by aiding movement or avoiding capture by mucus and thus in general extending the lifespan of sperm in the FRT and/or improving functionality. The disclosed methods and compositions can be employed in AI, for example, to reduce the number of sperm needed for insemination and to improve conception rates. Addition of proteins to cells other than sperm using the disclosed conjugates can also be used in other applications, such as transplantation protection.

In one aspect, the present disclosure provides conjugates that can be employed to improve the functionality of cells, such as sperm, by attaching a functional molecule of interest, such as a protein, to the surface of the cells. The disclosed conjugates comprise, or consist of, four components: a membrane anchoring component, such as a lipid; a spacer and/or solubilizing component, such as PEG; an attachment group or linker; and a functional molecule of interest that is attached to the spacer and/or solubilizing component via the attachment group.

Lipids that can be effectively employed in the disclosed conjugates include cholesterol, diacylglycerolipids, dialkylglycerolipids, glycerophospholipids, sphingosine derived diacyl- and dialkyl-lipids, ceramide, phosphatidate, phosphatidyl choline, phosphatidyl ethanolamine, phosphatidyl serine, phosphatidyl inositol and phosphatidyl glycerol. Examples of lipid-PEG-attachment group structures employed in the disclosed conjugates include those provided in FIGS. 1A-1C. In specific embodiments, the membrane anchoring component is cholesterol, the spacer and/or solubilizing component is PEG, the attachment group is an amine reactive group and the functional molecule of interest is catalase or glutathione.

In certain embodiments, functional molecules of interest employed in the disclosed conjugates are able to increase the lifespan of sperm in the FRT by at least 20%, 30%, 40% or 50% compared to untreated sperm. Examples of such molecules include, but are not limited to, amino acids and their derivatives, glutathione, CD55, CD59, CD73, SPAM1, DNaseI, catalase, and variants thereof. The amino acid sequences of CD55, CD59, CD73, SPAM1, DNase1L3 and catalase from bovine are provided in SEQ ID NO: 1-6, respectively. Seminal plasma proteins that bind to the surface of sperm or other sperm surface proteins can also be used as the functional molecules of interest employed in the disclosed conjugates. In certain embodiments, the functional molecules of interest are polypeptides selected from the group consisting of SEQ ID NO: 7-163 and variants thereof.

In another aspect, compositions comprising one or more of the conjugates disclosed herein and a physiologically acceptable carrier are also provided, together with preparations comprising at least one such composition and live sperm. In certain embodiments, the live sperm bear the X chromosome. Such preparations can be effectively employed in AI or in vitro fertilization.

In a further aspect, methods for improving the functionality and/or fertility of sperm are provided, such methods comprising contacting the sperm with an effective amount of a conjugate or composition disclosed herein. Such methods can be effectively employed with sperm from cows, pigs, sheep, goats, humans, camels, horses, deer, alpaca, dogs, cats, rabbits and rodents. In certain embodiments, the sperm are sorted into X and Y chromosome-bearing sperm either prior to or after contact with the conjugate or composition.

In yet another aspect, the present disclosure provides methods for making a preparation for use in AI or in vitro fertilization, such methods comprising obtaining sperm from a mammal, optionally separating the sperm into X chromosome-bearing and Y chromosome-bearing sperm, and contacting the sperm with an effective amount of a composition and/or conjugate disclosed herein. Methods for separating X chromosome-bearing sperm from Y chromosome-bearing sperm are known to those of skill in the art, and include, for example, flow cytometry. Such methods include, but are not limited to, those described in U.S. Pat. Nos. 5,135,759, 5,985,216, 6,149,867 and 6,263,745.

Methods for the cryopreservation of sperm are also provided by the present disclosure. Such methods comprise: (a) contacting the sperm with a cryoprotectant and an effective amount of a composition and/or conjugate disclosed herein, and (b) storing the sperm and the composition/construct at a temperature of about 4° C. to about −196° C., wherein the effective amount of the composition/conjugate is sufficient to increase the functionality and/or fertility of the sperm relative to sperm stored without the composition/conjugate. Examples of cryoprotectants that can be effectively employed in such methods include, but are not limited to, PEG, dimethyl sulfoxide (DMSO), ethylene glycol, propylene glycol, polyvinyl pyrrolidone (PVP), polyethylene oxide, raffinose, lactose, trehalose, melibiose, melezitose, mannotriose, stachyose, dextran, hydroxy-ethyl starch, sucrose, maltitol, lactitol and glycerol. In related aspects, preparations comprising cryogenically preserved sperm and a composition and/or conjugate disclosed herein are provided. Methods for cryopreserving sperm are well known in the art and include those disclosed, for example, in U.S. Pat. No. 7,208,265 and US Patent Application Publication no. US 2007/0092860.

The methods, compositions and constructs disclosed herein are particularly advantageous in the preparation of semen for use in AI of mammals including, but not limited to, cows, pigs, sheep, goats, humans, camels, horses, deer, alpaca, dogs, cats, rabbits and rodents. Semen used in such methods may be either fresh ejaculate or may have been previously frozen and subsequently thawed.

These and additional features of the present invention and the manner of obtaining them will become apparent, and the invention will be best understood, by reference to the following more detailed description.

### BRIEF DESCRIPTION OF THE DRAWINGS

FIGS. 1A-C show the structure of a tripartite molecule disclosed herein, with FIG. 1A showing the general structure, FIG. 1B showing the structure where X=NHS (ester amine reactive group) and FIG. 1C showing the structure where X=ester amine reactive group plus fluorescein.

FIGS. 2A-D illustrate binding of a cholesterol-PEG5000-FITC-catalase conjugate to sperm as determined by flow cytometry, with FIG. 2A showing flow cytometry analysis of freshly washed sperm, FIG. 2B showing flow cytometry analysis of sperm treated with catalase but without the cholesterol-PEG5000-FITC linker, FIG. 2C showing flow cytometry analysis of sperm treated with cholesterol-PEG5000-FITC-catalase, and FIG. 2D showing flow cytometry analysis of sperm treated with PBS alone. Results shown are live cells that were negative for Hoechst 33258.

FIGS. 3A-D illustrate binding of a cholesterol-PEG5000-FITC-catalase conjugate to Jurkat cells as determined by flow cytometry, with FIG. 3A showing flow cytometry analysis of untreated Jurkat cells, FIG. 3B showing flow cytometry analysis of Jurkat cells with catalase but without the cholesterol-PEG5000-FITC linker, FIG. 3C showing flow cytometry analysis of Jurkat cells treated with PBS alone, and FIG. 3D showing flow cytometry analysis of Jurkat cells with cholesterol-PEG5000-FITC-catalase. Results shown are live cells that were negative for Hoechst 33258.

### DETAILED DESCRIPTION

As outlined above, the present disclosure provides methods for improving the functionality and/or fertility of sperm, together with compositions and conjugates for use in such methods. In certain embodiments, the methods and compositions disclosed herein enhance sperm motility, protect sperm from phagocytosis, aid sperm in avoiding capture by mucus, extend the lifespan of sperm in the FRT, and/or enhance the function of a necessary sperm characteristic. The ability of a composition or conjugate to increase the functionality and/or fertility of sperm may be determined by contacting sperm with the composition or conjugate; measuring parameters such as the motility, resistance to neutrophil attack, membrane integrity and/or presence of sperm surface markers indicative of capacitation and acrosome status on the treated sperm and ability to recover from cryopreservation; and comparing the results with those obtained for untreated sperm. The functionality of sperm can also be determined by investigating their interaction with cervical mucus/explants or synthetic analogues, and/or their ability to capacitate, acrosome react and fertilize in vitro. Techniques for measuring the above parameters are well known in the art and include those described below. In certain embodiments, the disclosed methods comprise contacting the compositions and/or conjugates disclosed herein with either sorted or unsorted sperm.

### Conjugates

The present disclosure provides methods for adding a functional molecule of interest to the surface of cells, such as sperm, using a conjugate including a membrane anchoring agent, PEG and the molecule of interest. Such conjugates provide protection or enhancement of sperm functionality while at the same time allowing sperm to maintain the array of molecular and cellular interactions that occur in ascent through the FRT. In certain embodiments, the disclosed conjugates have the following general structure: membrane anchoring agent-PEG-X-functional molecule, where X is any reactive group (referred to herein as an attachment group) that allows conjugation of at least one functional molecule of interest.

As used herein, the term “membrane anchoring agent” or “membrane anchoring component” refers to a molecule that is known to spontaneously and stably incorporate into lipid bilayers, including cell membranes. Examples of such molecules include, but are not limited to, the synthetic molecules described in US Patent Publication no. US 2007/0197466, the disclosure of which is hereby incorporated by reference. In certain embodiments, the membrane anchoring agent is a lipid. Lipids that may be effectively employed in the disclosed methods include, but are not limited to, diacyl- and dialkylglycerolipids, including glycerophospholipids and sphingosine derived diacyl- and dialkyl lipids, including ceramide. In certain embodiments, the lipid is selected from the group consisting of: cholesterol, diacylglycerolipids, phosphatidate, phosphatidyl choline, phosphatidyl ethanolamine, phosphatidyl serine, phosphatidyl inositol and phosphatidyl glycerol. The lipid may be derived from one or more cis-desaturated fatty acids.

Cholesterol is considered to be a desirable lipid membrane anchoring agent as this lipid is the most abundant molecule in sperm surface membranes (Flesch et al., 2001) and is lost upon capacitation in the oviduct. Thus the same mechanism that removes endogenous cholesterol (cholesterol chelating agents such as bovine serum albumin; BSA) may also remove some of the added conjugate before fertilization.

PEGs having a wide range of lengths, or molecular weights, and a varying number of branches can be effectively employed in the disclosed conjugates. For example, in certain embodiments the PEG has a molecular weight in the range of about 200 to about 40,000 daltons. PEGs contemplated for use in the conjugates disclosed herein include, but are not limited to, PEGs having one or more amine reactive groups that allow conjugation to a protein, and include linear and branched chain PEGs. As will be appreciated by those of skill in the art, when a PEG having an amine reactive group is employed in the conjugate, the attachment group (X) is the amine reactive group.

In certain embodiments, the attachment group (X) is an amine reactive group, however the attachment group can be any group that reacts with —COOH, —OH and/or —SH groups as well as disulfide (—S—S—) bonds and oxidized carbohydrates, on proteins or small molecules (Greenwald et al., 2003; Roberts et al., 2002). Examples of reactive groups that have previously been used to attach PEG to proteins or peptides are shown in Table 1 below. Alternatively, the membrane anchoring agent-PEG backbone can be linked to the functional molecule using a biotin-streptavidin linkage or click chemistry (Lutz and Zarafshani, 2008).

TABLE 1
Examples of reactive groups used to attach PEG to specific groups on
proteins

| | Reactive group previously used to |
|---|---|
| | attach PEG to proteins (see Jevsevar |
| | et al., 2010; Roberts et al., 2002 and |
| Protein reactive target | references within) |
| Thiol (SH—R) | maleimide |
| Thiol (SH—R) | vinyl sulfone |
| Thiol (SH—R) | iodoacetamide |
| Thiol (SH—R) | orthopyridyl disulfide |
| Oxidized carbohydrate residue | hydrazide |
| Histidine residue | benzotriazole |
| Histidine residue | succinimidyl-carbonate |
| Amine group (H₂N—R) | succinimidyl active esters based on |
| | propionic and butanoic acids |

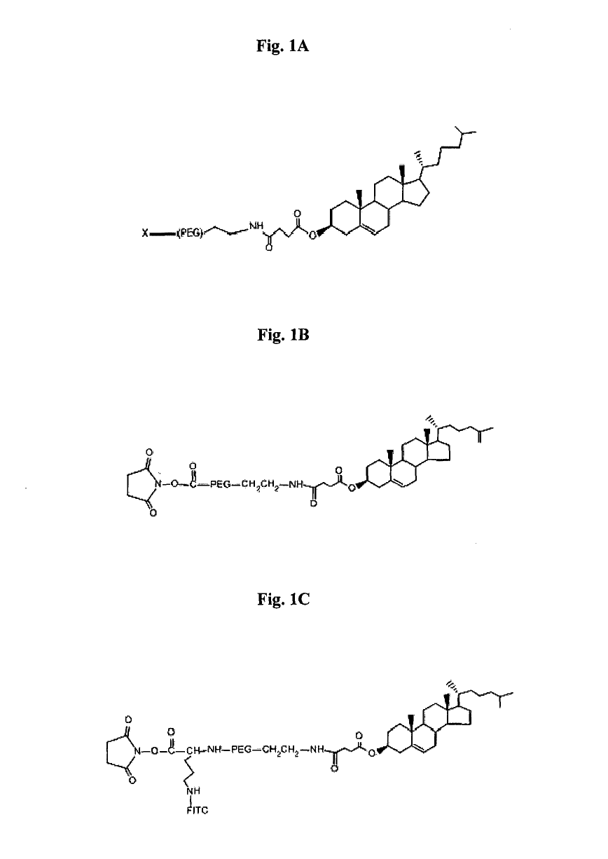

An example of the general structure of a cholesterol-PEG-attachment group starting tripartite molecule is shown in FIG. 1A, where X=any attachment group. Examples of specific starting tripartite molecules wherein X=NHS (ester amine reactive group) and X=ester amine reactive group plus fluorescein are shown in FIGS. 1B and 1C, respectively. Tripartite cholesterol-PEG-X molecules are available commercially and include, for example, those available from Nanocs Inc. The tripartite molecule is initially covalently attached via the attachment group to the functional molecule of interest, such as catalase. After attachment of the functional molecule of interest (e.g. catalase) and purification, the conjugate can be contacted with cells, such as sperm, whereby the conjugate binds to the surface of the sperm.

Functional molecules of interest that can be added to sperm using the disclosed conjugates include amino acids and their derivatives, polyamino acids, peptides, enzymes, adhesion molecules, immune proteins and antigens. Specific examples of functional molecules of interest include antioxidants such as catalase and glutathione. Catalase and glutathione both protect sperm from H₂O₂, and if sperm are exposed to H₂O₂ in the FRT, membrane attached oxidation protection would aid sperm motility.

Other examples of functional molecules that can be effectively employed in the disclosed methods and conjugates include molecules that potentially protect sperm from immune attack, including CD55 (decay factor), CD59 and CD73 (Kirchhoff and Hale, 1996); or entrapment by neutrophils, such as DNase1 (Alghamdi and Foster, 2005). Membrane bound CD55 and CD59 inhibit the formation of complement induced membrane attack complex and could protect cells or sperm from complement attack (Fraser et al., 2003). CD55 and CD59 are glycosylphosphatidylinositol (GPI) linked proteins present on the surface of sperm and have specific roles in sperm function (Donev et al., 2008). Addition of proteins such as CD55 and CD59 to the cell surface also has application in other uses, such as transplantation protection (Hill et al., 2006). DNase1 present in bovine seminal fluid is known to be associated with sperm fertility (Bellin et al., 1998; McCauley et al., 1999).

Other molecules that are important for fertility and that can also be employed in the disclosed methods include SPAM1, which is also GPI linked and present in the epididymis (Kirchhoff et al., 1997). SPAM1 is a potential sperm adhesion molecule and hyaluronidase that enables sperm to penetrate through the hyaluronic acid-rich cumulus cell layer surrounding the oocyte (Lathrop et al., 1990).

If sperm surface charge is important for movement of sperm through the FRT then altering surface charge or charge distribution could improve fertility. Using the conjugate described here and a functional group composed of amino acids, amino acid derivatives, polymeric amino acids or peptides enables sperm surface charge to be manipulated. For example, reacting an amine reactive cholesterol-PEG with glycine would allow addition of one negative charge per conjugate, reaction with glutamic acid adds two negative charges per conjugate, γ-carboxy-glutamic acid adds three negative charges per conjugate, and poly(L-glutamic acid) in defined numbers of residues, such as 20 or 50 (available from Almanda Polymers), allows addition of 21 or 51 negative charges per conjugate, respectively.

In general, apart from sperm charge modification, the seminal fluid (Novak et al., 2010a; Novak et al., 2010b; Rodriguez-Martinez et al., 2011) or epididymal proteins (Belleannee et al., 2011) that bind to sperm, or proteins present on sperm, and are correlated with high fertility (D'Amours et al., 2010; Novak et al., 2010b) represent functional molecules that can be employed in the disclosed methods and conjugates for addition to sperm.

The addition of GPI lipid anchored proteins to sperm during sperm maturation occurs at least partly through a mechanism where epididymosomes transfer such proteins to the sperm surface (Frenette et al., 2006; Kirchhoff and Hale, 1996). Epididymosomes are small membranous vesicles secreted by epithelial cells within the luminal compartment of the epididymis (Girouard et al., 2009). The inventors believe that the addition of a cholesterol-PEG-functional group construct to sperm is analogous to the transfer of such GPI-linked proteins that occurs in the epididymis.

### Polypeptides and Proteins

Proteins and/or polypeptides employed in the disclosed methods, compositions and conjugates can be isolated from seminal fluid (Kelly et al., 2006; Novak et al., 2010a; Novak et al., 2010b), epididymis or accessory sex glands (Moura et al., 2006a, 2007; Moura et al., 2006b) or other sources (e.g. catalase from bovine liver (Summer and Dounce, 1937)), or are commercially available. Alternatively, such proteins and/or polypeptides can be prepared recombinantly by inserting a polynucleotide that encodes the protein into an expression vector and expressing the antigen in an appropriate host. Any of a variety of expression vectors known to those of ordinary skill in the art may be employed. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, mycobacteria, insect, yeast or a mammalian cell line such as COS or CHO.

The proteins and/or polypeptides employed in the methods, compositions and conjugates disclosed herein are isolated and purified, as those terms are commonly used in the art. Preferably, the proteins and/or polypeptides are isolated to a purity of at least 80% by weight, more preferably to a purity of at least 95% by weight, and most preferably to a purity of at least 99% by weight. In general, such purification may be achieved using, for example, the standard techniques of ammonium sulfate fractionation, SDS-PAGE electrophoresis, and affinity chromatography.

The conjugates and compositions disclosed herein encompass variant polypeptide sequences that have been modified by one or more amino acid deletions, additions and/or substitutions. Variant sequences preferably exhibit at least 75%, more preferably at least 80%, more preferably at least 85%, more preferably at least 90%, more preferably yet at least 95%, and most preferably at least 98% identity to a specific polypeptide sequence disclosed herein. The percentage identity is determined by aligning the two sequences to be compared as described below, determining the number of identical residues in the aligned portion, dividing that number by the total number of residues in the inventive (queried) sequence, and multiplying the result by 100. In addition to exhibiting the recited level of sequence identity, variant sequences preferably exhibit a functionality that is substantially similar to the functionality of the specific sequences disclosed herein. Preferably a variant polypeptide sequence will have at least 80%, more preferably at least 85%, more preferably at least 90%, more preferably yet at least 95%, and most preferably 100% of the sperm fertility enhancing activity possessed by the specifically identified polypeptide sequence in one or more sperm fertility assays, such those described below. Such variants may generally be identified by modifying one of the polypeptide sequences disclosed herein, and evaluating the properties of the modified polypeptide using, for example, the representative procedures described herein.

In certain embodiments, variant sequences differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide in the conjugate.

Polypeptide sequences may be aligned, and percentages of identical amino acids in a specified region may be determined against another polypeptide, using computer algorithms that are publicly available, such as the BLASTP algorithm. BLASTX and FASTX algorithms compare nucleotide query sequences translated in all reading frames against polypeptide sequences. The use of the BLAST family of algorithms is described at NCBI's website and in the publications of Altschul et al. (Altschul et al., 1990; Altschul et al., 1997). The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

### Methods

In certain embodiments of the disclosed methods, sperm are purified by a single density layer (Percoll™ PLUS, GE Healthcare, see protocol below). Sperm are then incubated in a suitable media with an effective amount of one or more of the compositions and/or conjugates disclosed herein for a short period of time, followed optionally by the addition of a suitable extender to enable immediate use or freezing. Alternatively, the compositions and/or conjugates are added directly to the ejaculate and, after slight dilution, a short incubation (15-30 minutes) and the addition of extender, the resulting mixture is either cooled or frozen for storage. In another method, the compositions and/or conjugates are added to extended semen. In other embodiments, sperm are sexed by flow cytometry and are collected in media containing an effective amount of one or more of the disclosed compositions and/or conjugates. Alternatively, once sufficient sorted sperm are collected, the composition and/or conjugate is added and the resulting mixture is incubated in a suitable media for a short period of time, followed by the addition of extender and then either immediate use or freezing.

As used herein, the term “effective amount” of a composition and/or conjugates disclosed herein refers to that amount sufficient to enhance sperm motility, protect sperm from phagocytosis, allow sperm to avoid capture by mucus, extend the lifespan of sperm in the FRT, and/or increase sperm functionality by at least 5-50% compared to untreated sperm.

Those of skill in the art will appreciate that for use in the disclosed methods, the compositions and conjugates disclosed herein may be present in compositions including one or more physiologically acceptable carriers or diluents, such as water or saline. Such compositions may additionally contain other components, such as preservatives, stabilizers, buffers and the like. Carriers, diluents and other components suitable for use in the present compositions are well known to those of skill in the art and include those currently used in preparations for AI.

All U.S. patents, U.S. patent application publications, U.S. patent applications, foreign patents, foreign patent applications, non-patent publications, tables, sequences, web pages, or the like referred to in this specification, are incorporated herein by reference, in their entirety. The following examples are intended to illustrate, but not limit, this disclosure.

### EXAMPLES

### Example 1

### Preparation and Analysis of Cholesterol-PEG-Catalase

Equal volumes of 2 mM cholesterol-PEG5000-NHS-FITC (Nanocs) and 20 μM bovine catalase (Sigma; 100:1 ratio of cholesterol-PEG5000-NHS-FITC to bovine catalase) in phosphate buffered saline (PBS) were mixed by rotation for 3 hr at room temperature. The mixed solution was then dialysed into PBS using a 50 kDa molecular weight cut off (MWCO) membrane at 4° C. overnight. The free cholesterol-PEG5000-NHS-FITC that had not reacted with the catalase was removed using an ammonium sulphate precipitation where 200 μl of 4.1 M saturated ammonium sulphate solution was added slowly into 500 μl of the cholesterol-PEG5000-NHS-FITC/catalase mixture. Centrifugation of the sample mixture for 20 min at 20,000×g separated the free cholesterol-PEG5000-NHS-FITC as a pellet, and the reacted cholesterol-PEG5000-FITC-catalase as supernatant. Both supernatant and pellet were then dialysed into PBS using a 10 kDa MWCO membrane at 4° C. overnight. Dialysed samples were analysed by sodium dodecyl sulfate polyacrylamide gel electrophoresis (SDS-PAGE) under reducing conditions. The resulting gel was subjected to fluorescence imaging using an ImageQuant LAS-4000 (GE Healthcare), and stained with the Coomassie Brilliant Blue R-250 to visualize the protein bands. This combined gel analysis indicated that the catalase had been labeled with cholesterol-PEG and that the cholesterol-PEG that had not reacted with catalase was removed by precipitation (cholesterol-PEG-catalase>95% purity). The average number of cholesterol-PEG molecules added per catalase monomer was determined by measuring the fluorescence of known protein amount of cholesterol-PEG5000-NHS-FITC and employing a standard curve of free cholesterol-PEG5000-NHS-FITC concentration versus fluorescence. Depending upon the preparation of cholesterol-PEG-catalase, the range varied from 4.0 to 5.4 molecules of cholesterol-PEG per catalase monomer or 4× this number for the intact tetramer.

Recovery of the cholesterol-PEG5000-FITC-catalase was determined by Coomassie staining and catalase activity assay. For the catalase activity assay, 0.5 μl of test samples and serially diluted bovine catalase (Sigma) were placed in a microtitre plate, and incubated with 50 μl of 10 ng/ml of streptavidin-HRP (Biosource) and 50 μl of TMB substrate (Invitrogen) for 5 min. The plate was read at 450 nm on an EnVision plate reader (PerkinElmer) after stopping the reaction with 50 μl of 2 M sulfuric acid. Catalase activity of the test sample was calculated from the standard curve generated with known concentrations of bovine catalase. The catalase activity assay showed 85% recovery (equivalent to 1.11 mg; 44,406 units) of catalase in the form of cholesterol-PEG5000-FITC-catalase.

### Example 2

### Binding of Cholesterol-PEG-Catalase to Sperm

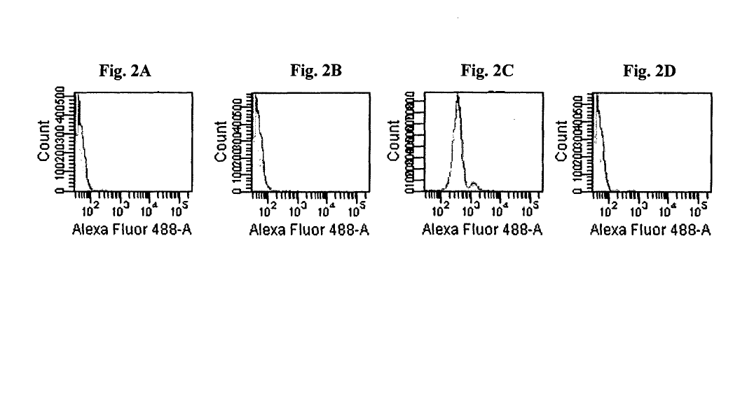

1.5 ml of bovine sperm in liquid extender was carefully placed on top of 4 ml of 60%_Percoll™ PLUS (GE Healthcare) column, and centrifugated for 20 min at 700×g at 20° C. The resulting purified sperm pellet was resuspended in non-capacitating media (NCM; see Table 2) containing 0.1 mg/ml of BSA to a cell concentration of 5×10⁷ ml. 200 μl of the purified sperm was mixed with an equal volume of 3 mg/ml cholesterol-PEG5000-FITC-catalase, and incubated light protected at 37° C. for 30 min. The unbound cholesterol-PEG5000-FITC-catalase was removed by layering 400 μl of sperm/cholesterol-PEG5000-FITC-catalase mixture on the top of 500 μl of 60% Percoll™ PLUS column, followed by centrifugation for 20 min at 700×g at 20° C. The sperm pellet was resuspended in NCM containing 0.1 mg/ml of BSA to a total volume of 400 μl. The binding of cholesterol-PEG5000-FITC-catalase to sperm was analysed by flow cytometry and catalase activity assay. For flow cytometry analysis, the cholesterol-PEG5000-FITC-catalase bound sperm were diluted to 5×10⁶ cells/ml in NCM containing 0.1 mg/ml of BSA, and stained with 0.2 μg/ml of a viability dye (Hoechst 33258). Significant binding (approx. 10 fold over background) of cholesterol-PEG5000-FITC-catalase to live sperm (Hoechst 33258 negative) was observed (see FIG. 2C). The catalase activity of cholesterol-PEG5000-FITC-catalase bound sperm was measured as described in Example 1. 79 units of catalase activity were measured in 2.5×10⁶ sperm cells after binding with cholesterol-PEG5000-FITC-catalase, whereas no catalase activity was detected in control sperm without addition of cholesterol-PEG5000-FITC-catalase.

TABLE 2
1x NCM (non-capacitating media, pH 7.4)

| Component | Concentration | |
|---|---|---|
| NaH₂PO₄ | 0.3 | mM |
| KCl | 3.1 | mM |
| MgCl₂ | 0.4 | mM |
| Sodium pyruvate | 1 | mM |
| HEPES | 40 | mM |
| NaCl | 100 | mM |
| Lactate (85%) | 21.7 | mM |
| Gentamicin | 50 | μg/ml |

### Example 3

### Binding of Cholesterol-PEG-Catalase to Jurkat Cells

Jurkat cells (ATCC, TIB-152; human T lymphocyte cell line) were cultured in RPMI-1640 media with 10% fetal calf serum. For analysis, cells were removed from culture, centrifuged at 700×g for 5 min, the culture media was removed and cells were resuspended at a concentration of 5×10⁷ cells/ml in PBS. 100 μl of the Jurkat cell suspension was mixed with 100 μl of 2 mg/ml cholesterol-PEG5000-FITC-catalase and incubated light protected at 37° C. for 30 min. Cells were centrifuged for 5 min at 700×g, supernatant removed, and resuspended in 400 μl of fresh PBS. The Jurkat cells were analyzed by both flow cytometry and catalase activity assay.

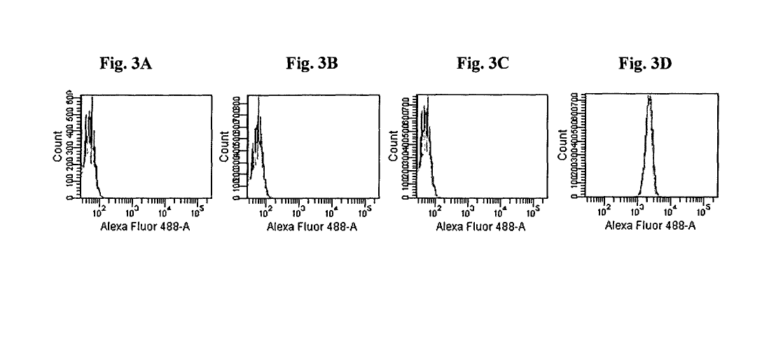

For flow cytometry analysis, the Jurkat cells were diluted in 200 μl of PBS to a concentration of 5×10⁶ cells/ml. 0.2 μg/ml of Hoechst 33258 (Invitrogen, H21491) was then added to each sample (see FIG. 3).

The catalase activity of the treated Jurkat cells was measured as described in Example 1. 50 μl of 5×10⁷ cells/ml of each test sample was analysed in the assay and the catalase activity contained in each of the Jurkat samples was calculated from the standard curve. The catalase activity assay indicated: 0 U of catalase in freshly washed Jurkat cells, 44 U in Jurkat cells with catalase alone without linker, 66 U in Jurkat cells with cholesterol-PEG5000-FITC-catalase (units per 2.5×10⁶ cells).

### Example 4

### Cholesterol-PEG-Catalase Protects Sperm from H₂O₂ Induced Loss of Motility

In this experimental configuration, a concentration of H₂O₂ is chosen that causes sperm to rapidly lose motility (30-60 minutes) unless oxidation protection like catalase is present. Sperm that have had cholesterol-PEG-catalase added and been washed so that no remaining free cholesterol-PEG-catalase remains are compared with sperm that have been exposed to a similar molar amount of catalase as contained in the cholesterol-PEG-catalase and then washed, and also with sperm exposed to no catalase, for their ability to resist H₂O₂ induced motility loss. Sperm motility is determined by a quantification system such as QualiSperm™ (Biophos).

### Example 5

### Identification of Seminal Plasma Proteins in Bovine Seminal Plasma

### a) Preparation of Bovine Seminal Plasma

Bovine semen samples were collected from three bulls using an artificial vagina and pooled. The pooled semen was centrifugated for 15 min at 1500×g to remove sperm cells and the resulting supernatant was further centrifugated for 15 min at 15000×g to remove any particulates. Complete mini protease inhibitor cocktail was added to the cleared seminal plasma before storage at −20° C. Protein concentration was measured by bicinchoninic acid (BCA) protein assay kit (Pierce).

### b) Peptide Preparation

Two methods were used to prepare peptides from bovine seminal plasma for proteomic analysis. The first method used standard in-solution digestion of proteins. Briefly, the seminal plasma was diluted in lysis buffer consisting of 7 M urea, 2 M thiourea, 4% CHAPS and 2 mM DTT, and incubated for 1 hr at 4° C. with constant rotation. Following centrifugation at 14000×g for 5 min at 4° C., an aliquot was removed for protein estimation by EZQ™ protein quantitation kit (Molecular Probes). 30 μl of cleared seminal plasma containing approximately 100 μg of proteins was alkylated for 30 min with 2-fold molar excess of iodoacetamide relative to the DTT. Proteins in the seminal plasma were then precipitated with methanol/chloroform. The resulting protein pellet was reconstituted in 0.5 M TEAB and 1 M urea containing 0.1 mg/ml trypsin, and incubated overnight at 37° C. Filter-aided sample preparation method (FASP II) (Wisniewski et al., 2009) was the second method used for peptide preparation from seminal plasma.

Acetonitrile was added to 5% to all peptide samples, before acidifying the peptides to pH 2 to 3 with formic acid. Peptides were then desalted on Sep-Pak Vac tC18 solid phase extraction cartridge (Waters), and completely dried in vacuum concentrator.

### c) Proteome Analysis

Dried peptide samples were sent to the Australian proteome analysis facility (APAF), provided by the Australian Government through the National Collaborative Research Infrastructure Strategy (NCRIS). At APAF, high sensitivity amino acid analysis was carried out to accurately measure the amount of peptides in each sample. Peptide samples were loaded onto a Capillary LC system coupled to an MS/MS instrument. The peptides were separated using a reverse phase C18 column and directly eluted into a Q-STAR mass spectrometer. 1D-LC-ESI-MS data acquired was analysed by ProteinPilot software 3.0 (ABI) to identify the proteins. A thorough identification search was conducted in ProteinPilot. The International Protein Index (IPI) Bos taurus database (v3.49) was used for all searches. Proteins identified from two samples prepared by two different peptide preparation methods were compared to each other. Ensembl genome browser (www.ensembl.org) was used to check the presence of transmembrane domains and signal peptide sequence in each identified protein.

A list of 73 seminal plasma proteins that may enhance functionality and/or fertility of sperm is provided in Table 3. The sequences for these proteins are provided in SEQ ID NO: 7-79, respectively. These 73 proteins were detected in two independently prepared seminal plasma samples, and also only those predicted to have signal peptide sequences were selected. Three seminal plasma proteins that were detected and that had multiple transmembrane domains were excluded from the list.

TABLE 3
Seminal plasma proteins detected by mass spectrometry and that also have
signal sequences

| Protein description | IPI identifier | Ensembl protein ID |
|---|---|---|
| MSLN MSLN protein | IPI00696375 | ENSBTAP00000000202 |
| GUSB GUSB protein | IPI00691968 | ENSBTAP00000000941 |
| ST3GAL1 Sialyltransferase 4A | IPI00692648 | ENSBTAP00000001538 |
| LTF Lactotransferrin | IPI00710664 | ENSBTAP00000001704 |
| LGALS3BP Galectin-3-binding protein | IPI00715562 | ENSBTAP00000001802 |
| PLOD1 Procollagen-lysine,2-oxoglutarate 5-dioxygenase 1 | IPI00718774 | ENSBTAP00000002658 |
| NUCB1 Nucleobindin-1 | IPI00722271 | ENSBTAP00000003073 |
| EFNA1 Ephrin-A1 | IPI00694962 | ENSBTAP00000004007 |
| PDC-109 Seminal plasma protein PDC-109 | IPI00715057 | ENSBTAP00000005073 |
| SERPINA5 Plasma serine protease inhibitor | IPI00686702 | ENSBTAP00000005307 |
| GLIPR1L1 GLIPR1-like protein 1 | IPI00713992 | ENSBTAP00000006002 |
| SPP1 Osteopontin | IPI00691887 | ENSBTAP00000006923 |
| CLU Clusterin | IPI00694304 | ENSBTAP00000007324 |
| MAN2B2 similar to mannosidase, alpha, class 2B, member 2 | IPI00709348 | ENSBTAP00000008532 |
| NGF Beta-nerve growth factor | IPI00685556 | ENSBTAP00000009796 |
| CFB Complement factor B (Fragment) | IPI00717527 | ENSBTAP00000009800 |
| SPADH2 spermadhesin 2 | IPI00696725 | ENSBTAP00000010565 |
| ENPEP Glutamyl aminopeptidase | IPI00685116 | ENSBTAP00000010972 |
| SERPINE2 Serpin peptidase inhibitor, clade E (Nexin, | IPI00692839 | ENSBTAP00000011485 |
| plasminogen activator inhibitor type 1), member 2 | | |
| CREG1 Cellular repressor of E1A-stimulated genes 1 | IPI00702458 | ENSBTAP00000011757 |
| ST6GAL1 Beta-galactoside alpha-2,6-sialyltransferase | IPI00692543 | ENSBTAP00000012565 |
| CCL2 C-C motif chemokine 2 | IPI00690357 | ENSBTAP00000013146 |
| SPADH1 Spermadhesin-1 | IPI00688936 | ENSBTAP00000014297 |
| TIMP2 Metalloproteinase inhibitor 2 | IPI00710784 | ENSBTAP00000014476 |
| ASAH1 Acid ceramidase | IPI00685320 | ENSBTAP00000014960 |
| NT5E 5~-nucleotidase | IPI00698673 | ENSBTAP00000015059 |
| OGN Mimecan | IPI00716123 | ENSBTAP00000015694 |
| B2M Beta-2-microglobulin | IPI00686769 | ENSBTAP00000016359 |
| STCH Heat shock 70 kDa protein 13 | IPI00685695 | ENSBTAP00000017155 |
| CRISP3 Cysteine-rich secretory protein 3 | IPI00715999 | ENSBTAP00000017167 |
| WFDC2 WAP four-disulfide core domain 2 | IPI00702630 | ENSBTAP00000018498 |
| CST6 Cystatin E/M | IPI00705340 | ENSBTAP00000019583 |
| PTGDS Prostaglandin-H2 D-isomerase | IPI00709683 | ENSBTAP00000020065 |
| VNN1 Pantetheinase | IPI00697935 | ENSBTAP00000020086 |
| TPP1 Tripeptidyl-peptidase 1 | IPI00721428 | ENSBTAP00000020469 |
| C15H11ORF34 Placenta-expressed transcript 1 protein | IPI00696232 | ENSBTAP00000020999 |
| TFPI2 Tissue factor pathway inhibitor 2 | IPI00709321 | ENSBTAP00000021062 |
| SCGB2A2 SCGB2A2 protein | IPI00711254 | ENSBTAP00000021195 |
| GAA Lysosomal alpha-glucosidase | IPI00695601 | ENSBTAP00000021325 |
| ARSA Arylsulfatase A | IPI00713745 | ENSBTAP00000021364 |
| VNN2 vanin 2 | IPI00698407 | ENSBTAP00000021759 |
| TEX101 TEX101 protein | IPI00694179 | ENSBTAP00000022313 |
| PPIB Peptidyl-prolyl cis-trans isomerase B | IPI00702098 | ENSBTAP00000022378 |
| CTSL2 Cathepsin L1 | IPI00687440 | ENSBTAP00000022710 |
| ALB Serum albumin | IPI00708398 | ENSBTAP00000022763 |
| CTSS Cathepsin S | IPI00702008 | ENSBTAP00000022774 |
| C3 Complement C3 (Fragment) | IPI00713505 | ENSBTAP00000022979 |
| NUCB2 Nucleobindin 2 | IPI00696729 | ENSBTAP00000023221 |
| DNASE1L3 deoxyribonuclease I-like 3 | IPI00696577 | ENSBTAP00000024347 |
| BSPH1 Seminal plasma protein BSP-30 kDa | IPI00709234 | ENSBTAP00000025134 |
| PLBD2 Putative phospholipase B-like 2 | IPI00702401 | ENSBTAP00000025343 |
| PLA2G7 Platelet-activating factor acetylhydrolase | IPI00699458 | ENSBTAP00000025719 |
| ANG Angiogenin-1 | IPI00710136 | ENSBTAP00000026126 |
| PIGR Isoform Long of Polymeric immunoglobulin receptor | IPI00696714 | ENSBTAP00000026377 |
| GSN Gelsolin | IPI00694255 | ENSBTAP00000026534 |
| ENPP3 Ectonucleotide pyrophosphatase/phosphodiesterase | IPI00712650 | ENSBTAP00000026900 |
| family member 3 | | |
| NPC2 Epididymal secretory protein E1 | IPI00711862 | ENSBTAP00000029271 |
| NPNT similar to Nephronectin precursor | IPI00826312 | ENSBTAP00000029938 |
| FAM3C FAM3C protein | IPI00714868 | ENSBTAP00000030039 |
| LOC525947 Serotransferrin-like | IPI00705493 | ENSBTAP00000031846 |
| RNASE1 Seminal ribonuclease | IPI00700712 | ENSBTAP00000036091 |
| AZGP1 Zinc-alpha-2-glycoprotein | IPI00698993 | ENSBTAP00000037042 |
| CTSA Lysosomal protective protein | IPI00687092 | ENSBTAP00000039003 |
| C1QTNF5 C1QTNF5 protein | IPI00692789 | ENSBTAP00000039816 |
| FUCA1 Tissue alpha-L-fucosidase | IPI00732644 | ENSBTAP00000040572 |
| NPPC C-type natriuretic peptide | IPI00714128 | ENSBTAP00000040960 |
| GPX5 glutathione peroxidase 5 | IPI00840765 | ENSBTAP00000042594 |
| SCGB1D2 Secretoglobin, family 1D, member 2 | IPI00824879 | ENSBTAP00000044006 |
| B4GALT4 UDP-Gal:betaGlcNAc beta 1,4- | IPI00690138 | ENSBTAP00000044479 |
| galactosyltransferase, polypeptide 4 | | |
| ACRBP similar to sp32 | IPI00716879 | ENSBTAP00000045557 |
| CDH1 CDH1 protein | IPI00711327 | ENSBTAP00000048482 |
| PEBP4 Phosphatidylethanolamine-binding protein 4 | IPI00693100 | ENSBTAP00000050912 |
| ACE 150 kDa protein | IPI00923883 | ENSBTAP00000053314 |

### Example 6

### Identification of Potential Soluble Surface and Single Pass Membrane Proteins on Bovine Sperm

### a) Purification of Bovine Sperm

200 μl of extended bovine semen was loaded onto 2 ml 50% Percoll™ PLUS column, and centrifugated at 1200×g for 20 min at room temperature. 5×10⁶ purified sperm cells resuspended in 1 ml of NCM (Table 2) were incubated with 10 μg/ml of biotinylated WGA (Vector Laboratories) for an hour at 28° C. on a rotating platform. 25 μl of washed streptavidin Dynabeads™ (Invitrogen) were then incubated with the sperm for an hour at 28° C. Dynabeads™/sperm complex was placed on magnet and washed three times with NCM. A minimal number of sperm was found in the supernatant, indicating that most sperm (>95%) formed a complex with the Dynabeads.

b) Peptide Preparation for iTRAQ Labeling

500 μl of lysis buffer containing 7 M urea, 2 M thiourea, 4% CHAPS and 13 mM DTT was added to the Dynabeads™/5×10⁶ sperm complex, vortexed, and incubated for 1 hr at 4° C. on a rotating platform. Dynabeads™/sperm complex was then removed by magnet, and the supernatant was centrifugated at 14000×g for 5 min to remove any remaining insoluble material. Protein estimation was performed using an EZQ™ protein quantitation kit (Molecular Probes). Filter-aided sample preparation method (FASP II) (Wisniewski et al., 2009) was then used to prepare peptides from the sperm lysate. Peptide samples were added to 5% acetonitrile, and acidified with formic acid to pH 2 to 3. Samples were then desalted on Sep-Pak Vac tC18 solid phase extraction cartridge (Waters), and completely dried in a vacuum concentrator.

c) iTRAQ Proteome Analysis

Dried peptide samples were sent to the APAF in Sydney for high sensitivity amino acid analysis and mass spectrometry analysis. Isobaric tags for relative and absolute quantitation (iTRAQ) was used for simultaneous identification and quantification of multiple peptide samples. 4-plex or 8-plex iTRAQ reagents (SCIEX) were used to analyse 4 or 8 different biological samples in a single experiment, respectively. In each iTRAQ experiment, an equal amount of each peptide sample was labeled with a different iTRAQ reagent. Mixed iTRAQ-labeled peptide sample was then loaded onto a strong cation ion exchange (SCX) column and fractionated into 20 fractions. Each fraction was separated by reverse-phase gradient and injected into a Q-STAR Elite mass spectrometer. 2D-nanoLC-ESI-MS/MS data acquired was then analysed by ProteinPilot software 3.0 (ABI), and relative quantitation and protein identification were obtained. Paragon algorithm was used to perform database matching for protein identification, protein grouping to remove abundant hits, and comparative quantitation. A thorough identification search was conducted in ProteinPilot. The IPI Bos taurus database (v3.49) was used for all searches. The data were normalized for loading error by bias correction using ProteinPilot. Proteins identified in multiple iTRAQ experiments were compared to each other, and also to the proteins identified in seminal plasma samples. Ensembl genome browser (www.ensembl.org) was used to check the presence of transmembrane domains and signal peptide sequence in each identified protein.

A list of 84 proteins in bovine sperm that may enhance sperm functionality and are likely to be on the sperm surface is provided in the Table 4. The sequences for these 84 proteins are provided in SEQ ID NO: 80-163, respectively. These proteins were selected from a total of 2206 proteins identified across 19 different sperm lysates by the following criteria: unique proteins with a signal sequence that occurred in at least two experiments and that were not listed in Table 3, and additionally, proteins with known mitochondrial subcellular location or more than one transmembrane domain were omitted.

TABLE 4
Likely sperm surface proteins excluding seminal plasma proteins

| Protein description | IPI identifier | Ensembl protein ID |
|---|---|---|
| RDH11 similar to retinol dehydrogenase 11 | IPI00694814 | ENSBTAP00000002535 |
| isoform 1 | | |
| GGH Gamma-glutamyl hydrolase | IPI00697223 | ENSBTAP00000009917 |
| PLBD1 Putative phospholipase B-like 1 | IPI00712643 | ENSBTAP00000020677 |
| SCGB1D2 LppAB | IPI00842256 | ENSBTAP00000044006 |
| PLBD1 PLBD1 protein | IPI00907129 | ENSBTAP00000050579 |
| TSBP TSBP protein | IPI00840484 | ENSBTAP00000001192 |
| MGC165862 MGC165862 protein | IPI00702545 | ENSBTAP00000005987 |
| NUP210L similar to nucleoporin 210 kDa-like | IPI00705819 | ENSBTAP00000006566 |
| ZPBP Zona pellucida binding protein | IPI00714900 | ENSBTAP00000007229 |
| LOC782909 similar to chromosome 9 open | IPI00702921 | ENSBTAP00000007827 |
| reading frame 79 | | |
| IZUMO1 similar to izumo sperm-egg fusion 1 | IPI00701171 | ENSBTAP00000015434 |
| SPACA1 Sperm acrosome membrane- | IPI00704953 | ENSBTAP00000025934 |
| associated protein 1 | | |
| CYB5R1 NADH-cytochrome b5 reductase 1 | IPI00689803 | ENSBTAP00000026548 |
| TMEM190 similar to Transmembrane protein | IPI00705075 | ENSBTAP00000028044 |
| 190 | | |
| LOC782834 63 kDa protein | IPI00709648 | ENSBTAP00000031240 |
| TSBP 63 kDa protein | IPI00906483 | ENSBTAP00000032083 |
| BSG RPE7 protein | IPI00696325 | ENSBTAP00000039862 |
| -293 kDa protein zonadhesin | IPI00840197 | ENSBTAP00000041534 |
| LOC100141230 similar to chromosome 9 | IPI00843355 | ENSBTAP00000041933 |
| open reading frame 79 | | |
| SPACA1 33 kDa protein | IPI00838870 | ENSBTAP00000044123 |
| LOC615968 similar to Acrosome formation- | IPI00815450 | ENSBTAP00000045709 |
| associated factor | | |
| ZPBP 41 kDa protein | IPI00837828 | ENSBTAP00000047419 |
| PAM Peptidyl-glycine alpha-amidating | IPI00842571 | ENSBTAP00000016466 |
| monooxygenase | | |
| HTATIP2 HIV-1 Tat interactive protein 2, | IPI00760398 | ENSBTAP00000017856 |
| 30 kDa | | |
| ADAM32 ADAM metallopeptidase domain | IPI00707155 | ENSBTAP00000031442 |
| 32 | | |
| LOC786878 Uncharacterized protein | IPI00717926 | ENSBTAP00000031847 |
| C9orf134 homolog | | |
| CD46 hypothetical LOC616002 | IPI00720452 | ENSBTAP00000041290 |
| ADAM3A 83 kDa protein | IPI00907064 | ENSBTAP00000044565 |
| LOC786599 similar to ADAM | IPI00823949 | ENSBTAP00000046987 |
| metallopeptidase domain 20 preproprotein | | |
| LOC530756 similar to acyltransferase like 1B | IPI00904029 | ENSBTAP00000048539 |
| LYZL6 Lysozyme-like protein 6 | IPI00715267 | ENSBTAP00000000032 |
| CRISP2 Cysteine-rich secretory protein 2 | IPI00699728 | ENSBTAP00000002805 |
| PPA2 Pyrophosphatase (Inorganic) 2 | IPI00714601 | ENSBTAP00000003165 |
| NUP155 similar to nucleoporin 155 kDa | IPI00710810 | ENSBTAP00000003193 |
| MFGE8 MFGE8 protein | IPI00689638 | ENSBTAP00000004272 |
| VSTM2A MGC142894 protein | IPI00694628 | ENSBTAP00000004727 |
| LOC780933; LOC615026 Cationic trypsin | IPI00706427 | ENSBTAP00000004737 |
| SPATA20 SPATA20 protein | IPI00689839 | ENSBTAP00000005441 |
| SPESP1 Sperm equatorial segment protein 1 | IPI00697380 | ENSBTAP00000007865 |
| C13H20ORF71 Short palate, lung and nasal | IPI00760496 | ENSBTAP00000008942 |
| epithelium carcinoma associated 3 protein | | |
| RUSC1 96 kDa protein | IPI00904174 | ENSBTAP00000009272 |
| CPVL similar to Carboxypeptidase, | IPI00706544 | ENSBTAP00000009404 |
| vitellogenic-like | | |
| RNASE6 Ribonuclease K6 | IPI00702961 | ENSBTAP00000011585 |
| ADAM2 Disintegrin and metalloproteinase | IPI00696982 | ENSBTAP00000012384 |
| domain-containing protein 2 | | |
| LYZL1 Lysozyme-like protein 1 | IPI00696700 | ENSBTAP00000013640 |
| FDPS Farnesyl pyrophosphate synthetase | IPI00839514 | ENSBTAP00000014472 |
| CTSF Cathepsin F | IPI00717812 | ENSBTAP00000014587 |
| NUDT9 NUDT9 protein | IPI00690450 | ENSBTAP00000015090 |
| PGCP Plasma glutamate carboxypeptidase | IPI00691920 | ENSBTAP00000015799 |
| HEXA Beta-hexosaminidase subunit alpha | IPI00702413 | ENSBTAP00000017261 |
| CPA1 89 kDa protein | IPI00843617 | ENSBTAP00000017727 |
| KLKBL4 KLKBL4 protein | IPI00702428 | ENSBTAP00000018804 |
| -Pyruvate dehydrogenase phosphatase | IPI00867405 | ENSBTAP00000021895 |
| regulatory subunit (Fragment) | | |
| PTI Pancreatic trypsin inhibitor | IPI00708836 | ENSBTAP00000023042 |
| LOC784519 similar to LOC512512 protein, | IPI00913657 | ENSBTAP00000024347 |
| partial | | |
| LYZL4 Lysozyme-like protein 4 | IPI00713792 | ENSBTAP00000024756 |
| -39 kDa protein GLI pathogenesis-related 1 | IPI00687877 | ENSBTAP00000025642 |
| like 2 | | |
| PLA2G7 Phospholipase A2, group VII | IPI00760435 | ENSBTAP00000025719 |
| GLB1L similar to galactosidase, beta 1-like | IPI00715275 | ENSBTAP00000027467 |
| HADHA FGF-2 binding protein | IPI00702650 | ENSBTAP00000032860 |
| -26 kDa protein | IPI00906471 | ENSBTAP00000033392 |
| MGC148336 MGC148336 protein | IPI00717678 | ENSBTAP00000035642 |
| MGC137014 Hibernation-associated plasma | IPI00689304 | ENSBTAP00000037834 |
| protein HP-20-like | | |
| APOB apolipoprotein B | IPI00710056 | ENSBTAP00000038799 |
| ZPBP2 Zona pellucida binding protein 2 | IPI00729769 | ENSBTAP00000040673 |
| LOC780846 Putative uncharacterized protein | IPI00686528 | ENSBTAP00000041742 |
| LOC780846 | | |
| LOC784495 LOC784495 protein | IPI00829561 | ENSBTAP00000042068 |
| LOC614476 Putative uncharacterized protein | IPI00694952 | ENSBTAP00000043576 |
| LOC614476 | | |
| -15 kDa protein | IPI00839329 | ENSBTAP00000044687 |
| ACRV1 Acrosomal vesicle protein 1 | IPI00712714 | ENSBTAP00000044695 |
| LOC786289 similar to signal-regulatory | IPI00904540 | ENSBTAP00000044718 |
| protein delta | | |
| SPP1 31 kDa protein | IPI00840962 | ENSBTAP00000044782 |
| NME4 Non-metastatic cells 4, protein | IPI00693558 | ENSBTAP00000045110 |
| expressed in | | |
| NDUFS6 NDUFS6 protein | IPI00883392 | ENSBTAP00000047840 |
| LOC615258 similar to mCG4550 isoform 2 | IPI00906659 | ENSBTAP00000050416 |
| LOC780846 28 kDa protein | IPI00904088 | ENSBTAP00000051552 |
| BSPH1 21 kDa protein | IPI00908264 | ENSBTAP00000052231 |
| SPAM1 Sperm adhesion molecule 1 | IPI00712321 | ENSBTAP00000006089 |
| PRCP Lysosomal Pro-X carboxypeptidase | IPI00698864 | ENSBTAP00000045060 |
| TTR Transthyretin | IPI00689362 | ENSBTAP00000014585 |
| AGA Aspartylglucosaminidase | IPI00693170 | ENSBTAP00000022716 |
| HINT2 Histidine triad nucleotide-binding | IPI00689717 | ENSBTAP00000015208 |
| protein 2 | | |
| ELSPBP1 similar to epididymal sperm | IPI00700508 | ENSBTAP00000021448 |
| binding protein E12 | | |
| CD59 CD59 molecule, complement regulatory | IPI00711804 | ENSBTAP00000002967 |
| protein | | |

### Example 7

### In Vitro Sperm Testing

A series of experiments are performed in vitro to determine the ability of a cholesterol-PEG-functional group conjugate to improve various measures of sperm functionality. Treated and untreated sperm are compared for changes in the following characteristics: motility; membrane integrity; mitochondrial membrane potential; membrane fluidity; chromatin integrity; lipid peroxidation; capacitation; acrosome reaction; binding of antibodies, heparin and lectins to the sperm surface (or modified sperm surface proteins); ability of sperm to migrate in the FRT; the resistance of sperm to phagocytosis; and the ability of sperm to fertilize in vitro (see Table 5 for details).

TABLE 5
In vitro sperm testing

| CHARACTERISTIC | ASSAY | NOTES | REFERENCES |
|---|---|---|---|
| Motility & | Qualisperm ™, Bright | Enables quick quantitative | (Tejerina et al., |
| morphology | field microscopy and | motility analysis for 1000s of | 2008) |
| | videomicroscopy with | cells. Can also indicate | |
| | image analysis | capacitation (hypermotility) | |
| Viability/ | Flow cytometry (FC)/ | Depending upon the experiment, | See (Gillan et |
| Membrane integrity | Fluorescent microscopy | different vital dyes are used | al., 2005) for a |
| | (FM) using a range of | depending upon their properties | review and |
| | dyes including | (all available from Invitrogen). | references |
| | Propidium iodide, Yo | These dyes are used alone but | within |
| | pro-1, Hoechst 33258 | also in combination with other FC | |
| | (H33258), | assays described below. Overall | |
| | LIVE/DEAD fixable far | allows quantification of cells with | |
| | red and SYBR 14 | permeant membranes | |
| Shape and | FC/FM | Enables quantification of size | (Gillan et al., |
| granularity | | and cellular aggregation changes | 2005) |
| Mitochondrial | FC/FM with DilC₁(5) | The DilC₁(5) dye is a member of | (Garner et al., |
| function/membrane | (Invitrogen), JC-1 | the cationic cyanine dyes that | 1997; Shapiro |
| potential | (Invitrogen), rhodamine | have been shown to accumulate in | et al., 1979) |
| | 123 (Sigma) | cells in response to membrane | |
| | | potential and thus permits | |
| | | quantification of mitochondrial | |
| | | functionality change. JC-1 and | |
| | | rhodamine 123 operates in a | |
| | | similar manner to DilC₁(5) | |
| Capacitation status | FC in combination with | Capacitation induces sperm | (Gadella and |
| | WGA-fluorescein | surface changes. WGA/Annexin | Harrison, 2002; |
| | (Invitrogen)/Annexin | V and merocyanin 540 all enable | Mahmoud and |
| | V-fluorescein/ | quantification of capacitation | Parrish, 1996; |
| | merocyanin 540 | changes | Medeiros and |
| | binding. Also the | | Parrish, 1996; |
| | ability to undergo | | Rathi et al., |
| | acrosome reaction is | | 2001) |
| | used as a measure of | | |
| | capacitation | | |
| Acrosome integrity | FC in combination with | Both PNA and SBTI allow | (Harper et al., |
| | PNA-647 (Invitrogen)/ | quantification of changes on the | 2008; Nagy et |
| | SBTI (Soyabean trypsin | sperm acrosome surface that | al., 2003) |
| | inhibitor)-488 | reflect acrosome reaction, | |
| | (Invitrogen) | although this assay is generally | |
| | | used to monitor spontaneous | |
| | | acrosome reaction. The ability of | |
| | | cells to acrosome react when | |
| | | initiated by calcium ionophore | |
| | | A23187 is also used as a measure | |
| | | of capacitation. | |
| Surface antibody | FC/FM in combination | We have developed polyclonal | |
| binding | with antibodies | antibodies to four seminal plasma | |
| | | proteins on bovine sperm (PDC- | |
| | | 109, BSP-A3, BSP-30 kDa, | |
| | | aSFP). These allow quantification | |
| | | of changes on the surface of | |
| | | sperm | |
| Lectin binding | FC/FM in combination | Enables quantification of changes | |
| | with lectins | to sperm and sperm surface | |
| | | proteins | |
| Heparin binding | FC/FM with fluorescent | Enables quantification of changes | (Dapino et al., |
| | heparin (Invitrogen) | in heparin binding to sperm and | 2006) |
| | | sperm surface proteins | |
| Anandamide and | Capacitation/motility/ | Agonists and antagonists of | (Gervasi et al., |
| related compounds | viability and acrosome | Cannabinoid receptors (CB1R | 2009; |
| interaction with | reaction | and CB2R) modify sperm | Maccarrone, |
| sperm | | characteristics in vitro and may be | 2009; |
| | | involved in the regulation and | Maccarrone et |
| | | activation of capacitation | al., 2005) |
| Membrane fluidity | FC with merocyanine | Enables quantification of changes | (Williamson et |
| | 540 | in membrane fluidity | al., 1983) |
| Assessment of | Acridine orange sperm | Enables quantification of | (Ballachey et |
| chromatin integrity | chromatin structure | chromatin integrity changes | al., 1988; |
| | assay (SCSA; uses FC) | | Evenson et al., |
| | | | 1980) |
| Sperm migration | Measure sperm | Enables quantification of sperm | (Aitken et al., |
| assay in cervical | migration in glass | motility in a media that resembles | 1992; Gillan et |
| mucus | capillary tube with | at least part of the FRT | al., 2008) |
| | fluorescent labeled | | |
| | sperm (H33342) | | |
| Neutrophil | Microscopic | Enables quantification of sperm | (Alghamdi et |
| phagocytosis assay | observation of | phagocytosis by neutrophils | al., 2009; |
| | phagocytes and | | Woelders and |
| | fluorescent labeled | | Matthijs, 2001) |
| | sperm (H33342) | | |
| Oviduct explants | Binding of fluorescent | Assays enable quantification of | (Ignotz et al., |
| binding/Annexin-II | labeled sperm (H33342) | ability of sperm to bind to oviduct | 2007; Teijeiro |
| binding | to oviduct. FC in | or the likely receptor on the | et al., 2009; |
| | combination with | oviduct | Waberski et al., |
| | fluorescent labeled | | 2005) |
| | Annexin-II protein | | |
| Lipid peroxidation | FC/FM with C11- | Enables quantification of | (Brouwers and |
| | BODIPY(581/591) | membrane lipid peroxidation | Gadella, 2003) |
| | (Invitrogen) | | |
| Oxidative stress to | FC with specific | Enables quantification of DNA | (De Iuliis et al., |
| DNA | antibody to 8-hydroxy- | damage caused by oxidative | 2009) |
| | 2′-deoxyguanosine | stress | |
| | (Biotrin International | | |
| | ltd) | | |
| ROS generation | FC with | Enables quantification of ROS | (Bass et al., |
| | Dihydroethidium (a | species | 1983; |
| | probe to detect | | Fridovich, |
| | superoxide) and 22,72- | | 1997; Guthrie |
| | dichlorodihydro- | | and Welch, |
| | fluorescein | | 2006) |
| | diacetate (a probe to | | |
| | detect hydrogen | | |
| | peroxide) | | |
| In vitro fertilization | Do with titration of | Enables overall quantification of | (Amann and |
| | sperm | the ability of sperm to bind zona | Hammerstedt, |
| | | pellucida, penetrate zona | 2002; Lu and |
| | | pellucida and fertilize the oocyte | Seidel, 2004; |
| | | | Saeki et al., |
| | | | 1995) |

### Example 8

### Sperm Maturation Model

In this model, as detailed below, bovine sperm are incubated overnight in NCM under non-capacitating conditions (simulating the conditions sperm experience for the majority of the journey in the FRT, starting cell viability approximately 90%). Following overnight incubation, sperm are diluted in capacitating media (CM; Table 6), triggering capacitation with high efficiency and minor loss of viability (cell viability in the 75-85% range). In typical experiments, when bovine sperm are capacitated with caffeine, db-cAMP and IBMX (3-isobutyl-1-methylxanthine), greater than 95% of viable cells capacitate as assessed by WGA-fluorescein/Annexin V or merocyanine 540 binding (see Table 5; WGA staining is the most sensitive, with approximately 10-fold shift in the staining upon capacitation). When cells are capacitated in vitro they also gain the capacity to acrosome react (Table 5). Although the combination of caffeine, db-cAMP and IBMX is an efficient inducer of capacitation, when more in vivo like capacitation induction is required, heparin is used. Sperm treated with cholesterol-PEG-functional molecule are compared with untreated sperm for their ability to capacitate, in particular using heparin induction method.

TABLE 6
1x CM (capacitating media, pH 7.4)

| Component | Concentration | |
|---|---|---|
| NaH₂PO₄ | 0.3 | mM |
| KCl | 3.1 | mM |
| MgCl₂ | 0.4 | mM |
| Sodium pyruvate | 1 | mM |
| HEPES | 20 | mM |
| NaCl | 100 | mM |
| Lactate (85%) | 21.7 | mM |
| NaHCO₃ | 60 | mM |
| CaCl₂ | 3.9 | mM |
| Gentamicin | 50 | μg/ml |
| Fatty acid-poor BSA | 2 | mg/ml |

### a) Day 1—Bovine Sperm Purification Using Percoll™ PLUS

A 90% Percoll™ PLUS solution is made by adding 10×NCM to Percoll™ PLUS. A 60% single layer gradient is then made by dilution with 1×NCM. In the standard method, 4 ml of 60% Percoll™ PLUS/NCM is added in a 15 ml tube, 1.5 ml of ejaculate in liquid extender (standard tris-egg yolk, extension 1:4 egg yolk-citrate-glycerol) is then gently loaded on top, and centrifuged at 700×g for 20 min at room temperature. The pellet is removed and washed once in 8 ml of NCM by centrifugation for 5 min at 700×g. The supernatant is then removed and the pellet resuspended in 1 ml of NCM. Capacitation treatment tubes are set up at a sperm concentration of 5×10⁷ cells/ml.

### b) Day 1—Flow Cytometry Analysis

Samples are prepared for flow cytometry analysis as follows. The components shown in Table 7 below are incubated with 5×10⁵ Percoll™ PLUS-purified bovine sperm in a final volume of 200 μl at room temperature for 10 min, while propidium iodide (PI) is added just before analyzing by flow cytometry.

TABLE 7

| FLUORESCENT | FINAL | | |
|---|---|---|---|
| COMPONENT | CONCENTRATION | | SUPPLIER |
| Propidium iodide | 0.3 | μM | Invitrogen |
| PNA-alexa fluor | 0.25 | μg/ml | Invitrogen |
| 647 | | | |
| SBTI-alexa fluor | 0.01 | μg/ml | Invitrogen |
| 488 | | | |
| WGA-fluorescein | 0.00625 | μg/ml | Invitrogen |
| or WGA-alexa | | | |
| fluor 647 | | | |

### c) Day 1—Incubation of Bovine Sperm Sample Overnight

Percoll™ PLUS-purified bovine sperm at 5×10⁷ cells/ml concentration are incubated in NCM overnight in a 28° C. water bath. The sperm are then visually assessed under inverted bright field microscope and/or using QualiSperm prior to inducing capacitation.

d) Day 2—Transition of Cells from Non-Capacitating Media to Capacitating Media

After overnight incubation, the cells are diluted in to CM (Table 2). Specifically, 1 ml of overnight incubated sperm is diluted 1:1 with 1 ml of CM media. Activators for capacitation, specifically caffeine and db-cAMP are added at a final concentration of 1 mM (˜16 hours after incubation started), and IBMX is added at a final concentration of 100 mM. Alternatively, bovine sperm capacitation is induced using heparin or methylbeta cyclodextrin (cholesterol acceptor). Samples are then incubated for an hour at 37° C.

### e) Day 2—Flow Cytometry Analysis of Capacitated Sperm

Similar to day 1, bovine sperm samples are then incubated with fluorescently labeled SBTI, PNA and WGA for 10 min and PI added just prior to flow analysis.

### Example 9

### In Vivo Field Artificial Insemination Trials

Achieving pregnancy is dependent upon both the male and female fertility, and also upon other factors (such as management of animals, parity, age, environment, insemination procedure etc.) and thus analysis of male fertility usually requires large numbers of animals in trials (Amann and Hammerstedt, 2002). At least for the bovine, the large number of sperm/ejaculate and also careful study design mean that many sources of variation can be controlled. In cattle, AI trials have been conducted to look at number of sperm required for insemination either alone (Den Daas et al., 1998) or in conjunction with other variables such as flow cytometry sorting (Bodmer et al., 2005), extender composition or other modification (Amann et al., 1999). The basic design is a sperm dose response using several bulls and a large number of cows (Den Daas et al., 1998).

In alternative studies, heterospermic inseminations with mixtures of treated and non-treated (control) sperm are employed to quickly determine functionality and/or fertility of the treated sperm. In this experimental design, two distinguishable types of sperm are inseminated simultaneously, with the aim being to compare the different types of sperm and thus remove female fertility as an experimental variable. Previous reports have described heterospermic insemination using sperm from multiple bulls (Dziuk, 1996; Flint et al., 2003), and a few methods have been developed together with various techniques to assess the success of the sperm (Flint et al., 2003; Parrish and Foote, 1985).

In specific studies, semen is collected from a single bull and sperm are either treated with a cholesterol-PEG-functional molecule or left untreated. Treated and untreated (control) sperm are labelled with two different fluorescent dyes (such as Hoechst 33342 and Vybrant DyeCycle stains) to enable the control and treated sperm to be distinguished. Equal amounts of the treated and control sperm are then simultaneously inseminated into the same cow, and reciprocal studies are also carried out to ensure effects on sperm transport are not due to the marker fluorescent dye. Twelve to sixteen hours after heterospermic insemination the cow is slaughtered, the uterus and oviduct removed, and the ratio of treated and control sperm in the upper uterine horn and oviduct is determined. Significantly increased number of treated sperm are present in the upper uterine horn and oviduct compared to untreated (control) sperm when the treatment successfully improves sperm functionality.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims appended hereto.

SEQ ID NO: 1-163 are set out in the attached Sequence Listing. The codes for nucleotide sequences used in the attached Sequence Listing, including the symbol “n,” conform to WIPO Standard ST.25 (1998), Appendix 2, Table 1.

### BIBLIOGRAPHY

Abuchowski, A., McCoy, J. R., Palczuk, N. C., van Es, T., and Davis, F. F. (1977a). Effect of covalent attachment of polyethylene glycol on immunogenicity and circulating life of bovine liver catalase. J Biol Chem 252, 3582-3586.
Abuchowski, A., van Es, T., Palczuk, N. C., and Davis, F. F. (1977b). Alteration of immunological properties of bovine serum albumin by covalent attachment of polyethylene glycol. J Biol Chem 252, 3578-3581.

Aitken, R. J., Bowie, H., Buckingham, D., Harkiss, D., Richardson, D. W., and West, K. M. (1992). Sperm penetration into a hyaluronic acid polymer as a means of monitoring functional competence. J Androl 13, 44-54.
Alghamdi, A. S., and Foster, D. N. (2005). Seminal DNase frees spermatozoa entangled in neutrophil extracellular traps. Biol Reprod 73, 1174-1181.
Alghamdi, A. S., Lovaas, B. J., Bird, S. L., Lamb, G. C., Rendahl, A. K., Taube, P. C., and Foster, D. N. (2009). Species-specific interaction of seminal plasma on sperm-neutrophil binding. Anim Reprod Sci 114, 331-344.
Altschul, S. F., Gish, W., Miller, W., Myers, E. W., and Lipman, D. J. (1990). Basic local alignment search tool. J Mol Biol 215, 403-410.
Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res 25, 3389-3402.
Alvarez, J. G., and Storey, B. T. (1985). Spontaneous lipid peroxidation in rabbit and mouse epididymal spermatozoa: dependence of rate on temperature and oxygen concentration. Biol Reprod 32, 342-351.
Amann, R. P., and Hammerstedt, R. H. (2002). Detection of differences in fertility. J Androl 23, 317-325.
Amann, R. P., Seidel, G. E., Jr., and Brink, Z. A. (1999). Exposure of thawed frozen bull sperm to a synthetic peptide before artificial insemination increases fertility. J Androl 20, 42-46.
Awda, B. J., Mackenzie-Bell, M., and Buhr, M. M. (2009). Reactive Oxygen Species and Boar Sperm Function. Biol Reprod.
Ballachey, B. E., Evenson, D. P., and Saacke, R. G. (1988). The sperm chromatin structure assay. Relationship with alternate tests of semen quality and heterospermic performance of bulls. J Androl 9, 109-115.
Bass, D. A., Parce, J. W., Dechatelet, L. R., Szejda, P., Seeds, M. C., and Thomas, M. (1983). Flow cytometric studies of oxidative product formation by neutrophils: a graded response to membrane stimulation. J Immunol 130, 1910-1917.
Baumber, J., Ball, B. A., Gravance, C. G., Medina, V., and Davies-Morel, M. C. (2000). The effect of reactive oxygen species on equine sperm motility, viability, acrosomal integrity, mitochondrial membrane potential, and membrane lipid peroxidation. J Androl 21, 895-902.
Bell, M., Sikka, S., Rajasekaran, M., and Hellstrom, W. (1992). Time course of hydrogen peroxide induced changes in the lipid peroxidation of human sperm membranes. Adv Contracept Deliv Syst 8, 144-150.
Belleannee, C., Belghazi, M., Labas, V., Teixeira-Gomes, A. P., Gatti, J. L., Dacheux, J. L., and Dacheux, F. (2011). Purification and identification of sperm surface proteins and changes during epididymal maturation. Proteomics 11, 1952-1964.
Bellin, M. E., Oyarzo, J. N., Hawkins, H. E., Zhang, H., Smith, R. G., Forrest, D. W., Sprott, L. R., and Ax, R. L. (1998). Fertility-associated antigen on bull sperm indicates fertility potential. J Anim Sci 76, 2032-2039.
Bienert, G. P., Schjoerring, J. K., and Jahn, T. P. (2006). Membrane transport of hydrogen peroxide. Biochim Biophys Acta 1758, 994-1003.
Bilodeau, J. F., Blanchette, S., Cormier, N., and Sirard, M. A. (2002). Reactive oxygen species-mediated loss of bovine sperm motility in egg yolk Tris extender: protection by pyruvate, metal chelators and bovine liver or oviductal fluid catalase. Theriogenology 57, 1105-1122.
Bodmer, M., Janett, F., Hassig, M., den Daas, N., Reichert, P., and Thun, R. (2005). Fertility in heifers and cows after low dose insemination with sex-sorted and non-sorted sperm under field conditions. Theriogenology 64, 1647-1655.
Brouwers, J. F., and Gadella, B. M. (2003). In situ detection and localization of lipid peroxidation in individual bovine sperm cells. Free Radic Biol Med 35, 1382-1391.
Calvete, J. J., and Sanz, L. (2007). Insights into structure-function correlations of ungulate seminal plasma proteins. Soc Reprod Fertil Suppl 65, 201-215.
Chen, A. M., and Scott, M. D. (2001). Current and future applications of immunological attenuation via pegylation of cells and tissue. BioDrugs 15, 833-847.
Chen, Q., and Duan, E. K. (2011). Aquaporins in sperm osmoadaptation: an emerging role for volume regulation. Acta Pharmacol Sin 32, 721-724.
Chen, Q., Peng, H., Lei, L., Zhang, Y., Kuang, H., Cao, Y., Shi, Q. X., Ma, T., and Duan, E. (2011). Aquaporin3 is a sperm water channel essential for postcopulatory sperm osmoadaptation and migration. Cell Res 21, 922-933.
D'Amours, O., Frenette, G., Fortier, M., Leclerc, P., and Sullivan, R. (2010). Proteomic comparison of detergent-extracted sperm proteins from bulls with different fertility indexes. Reproduction 139, 545-556.
Dapino, D. G., Marini, P. E., and Cabada, M. O. (2006). Effect of heparin on in vitro capacitation of boar sperm. Biol Res 39, 631-639.
De luliis, G. N., Thomson, L. K., Mitchell, L. A., Finnie, J. M., Koppers, A. J., Hedges, A., Nixon, B., and Aitken, R. J. (2009). DNA damage in human spermatozoa is highly correlated with the efficiency of chromatin remodeling and the formation of 8-hydroxy-2′-deoxyguanosine, a marker of oxidative stress. Biol Reprod 81, 517-524.
de Lamirande, E., and Gagnon, C. (1995). Impact of reactive oxygen species on spermatozoa: a balancing act between beneficial and detrimental effects. Hum Reprod 10 Suppl 1, 15-21.
Den Daas, J. H., De Jong, G., Lansbergen, L. M., and Van Wagtendonk-De Leeuw, A. M. (1998). The relationship between the number of spermatozoa inseminated and the reproductive efficiency of individual dairy bulls. J Dairy Sci 81, 1714-1723. Donev, R. M., Sivasankar, B., Mizuno, M., and Morgan, B. P. (2008). The mouse complement regulator CD59b is significantly expressed only in testis and plays roles in sperm acrosome activation and motility. Mol Immunol 45, 534-542.
Drobnis, E. Z., and Overstreet, J. W. (1992). Natural history of mammalian spermatozoa in the female reproductive tract. Oxf Rev Reprod Biol 14, 1-45.
Dziuk, P. J. (1996). Factors that influence the proportion of offspring sired by a male following heterospermic insemination. Animal Reproduction Science 43, 65-88.
Evenson, D. P., Darzynkiewicz, Z., and Melamed, M. R. (1980). Relation of mammalian sperm chromatin heterogeneity to fertility. Science 210, 1131-1133.
Flesch, F. M., Brouwers, J. F., Nievelstein, P. F., Verkleij, A. J., van Golde, L. M., Colenbrander, B., and Gadella, B. M. (2001). Bicarbonate stimulated phospholipid scrambling induces cholesterol redistribution and enables cholesterol depletion in the sperm plasma membrane. J Cell Sci 114, 3543-3555.
Flint, A. F., Chapman, P. L., and Seidel, G. E., Jr. (2003). Fertility assessment through heterospermic insemination of flow-sorted sperm in cattle. J Anim Sci 81, 1814-1822.
Foote, R. H. (2002). The history of artificial insemination: Selected notes and notables. Journal of Animal Science 80, 1-10
Fraser, D. A., Harris, C. L., Williams, A. S., Mizuno, M., Gallagher, S., Smith, R. A., and Morgan, B. P. (2003). Generation of a recombinant, membrane-targeted form of the complement regulator CD59: activity in vitro and in vivo. J Biol Chem 278, 48921-48927.
Frenette, G., Girouard, J., and Sullivan, R. (2006). Comparison between epididymosomes collected in the intraluminal compartment of the bovine caput and cauda epididymidis. Biol Reprod 75, 885-890.
Fridovich, I. (1997). Superoxide anion radical (O2−.), superoxide dismutases, and related matters. J Biol Chem 272, 18515-18517.
Frijters, A. C., Mullaart, E., Roelofs, R. M., van Hoorne, R. P., Moreno, J. F., Moreno, O., and Merton, J. S. (2009). What affects fertility of sexed bull semen more, low sperm dosage or the sorting process? Theriogenology 71, 64-67.
Gadella, B. M., and Harrison, R. A. (2002). Capacitation induces cyclic adenosine 3′,5′-monophosphate-dependent, but apoptosis-unrelated, exposure of aminophospholipids at the apical head plasma membrane of boar sperm cells. Biol Reprod 67, 340-350.
Garner, D. L., Thomas, C. A., Joerg, H. W., DeJarnette, J. M., and Marshall, C. E. (1997). Fluorometric assessments of mitochondrial function and viability in cryopreserved bovine spermatozoa. Biol Reprod 57, 1401-1406.
Gervasi, M. G., Rapanelli, M., Ribeiro, M. L., Farina, M., Billi, S., Franchi, A. M., and Perez Martinez, S. (2009). The endocannabinoid system in bull sperm and bovine oviductal epithelium: role of anandamide in sperm-oviduct interaction. Reproduction 137, 403-414.
Gillan, L., Evans, G., and Maxwell, W. M. (2005). Flow cytometric evaluation of sperm parameters in relation to fertility potential. Theriogenology 63, 445-457.
Gillan, L., Kroetsch, T., Maxwell, W. M., and Evans, G. (2008). Assessment of in vitro sperm characteristics in relation to fertility in dairy bulls. Anim Reprod Sci 103, 201-214.
Girouard, J., Frenette, G., and Sullivan, R. (2009). Compartmentalization of proteins in epididymosomes coordinates the association of epididymal proteins with the different functional structures of bovine spermatozoa. Biol Reprod 80, 965-972.
Greenwald, R. B., Choe, Y. H., McGuire, J., and Conover, C. D. (2003). Effective drug delivery by PEGylated drug conjugates. Adv Drug Deliv Rev 55, 217-250.
Guthrie, H. D., and Welch, G. R. (2006). Determination of intracellular reactive oxygen species and high mitochondrial membrane potential in Percoll-treated viable boar sperm using fluorescence-activated flow cytometry. J Anim Sci 84, 2089-2100.
Harper, C. V., Cummerson, J. A., White, M. R., Publicover, S. J., and Johnson, P. M. (2008). Dynamic resolution of acrosomal exocytosis in human sperm. J Cell Sci 121, 2130-2135.
Hawk, H. W. (1983). Sperm survival and transport in the female reproductive tract. J Dairy Sci 66, 2645-2660.
Hill, A., Ridley, S. H., Esser, D., Oldroyd, R. G., Cullen, M. J., Kareclas, P., Gallagher, S.,
Smith, G. P., Richards, S. J., White, J., et al. (2006). Protection of erythrocytes from human complement-mediated lysis by membrane-targeted recombinant soluble CD59: a new approach to PNH therapy. Blood 107, 2131-2137.
Hunter, R. H. (2003). Advances in deep uterine insemination: a fruitful way forward to exploit new sperm technologies in cattle. Anim Reprod Sci 79, 157-170.
Ignotz, G. G., Cho, M. Y., and Suarez, S. S. (2007). Annexins are candidate oviductal receptors for bovine sperm surface proteins and thus may serve to hold bovine sperm in the oviductal reservoir. Biol Reprod 77, 906-913.
Jackson, C. J., Charlton, J. L., Kuzminski, K., Lang, G. M., and Sehon, A. H. (1987). Synthesis, isolation, and characterization of conjugates of ovalbumin with monomethoxypolyethylene glycol using cyanuric chloride as the coupling agent. Anal Biochem 165, 114-127.
Jevsevar, S., Kunstelj, M., and Porekar, V. G. (2010). PEGylation of therapeutic proteins. Biotechnol J 5, 113-128.
Johnson, L. A., Flook, J. P., and Hawk, H. W. (1989). Sex preselection in rabbits: live births from X and Y sperm separated by DNA and cell sorting. Biol Reprod 41, 199-203.
Jones, R., Mann, T., and Sherins, R. (1979). Peroxidative breakdown of phospholipids in human spermatozoa, spermicidal properties of fatty acid peroxides, and protective action of seminal plasma. Fertil Steril 31, 531-537.
Katz, D. F., Drobnis, E. Z., and Overstreet, J. W. (1989). Factors regulating mammalian sperm migration through the female reproductive tract and oocyte vestments. Gamete Res 22, 443-469.
Kelly, V. C., Kuy, S., Palmer, D. J., Xu, Z., Davis, S. R., and Cooper, G. J. (2006). Characterization of bovine seminal plasma by proteomics. Proteomics 6, 5826-5833.
Kirchhoff, C., and Hale, G. (1996). Cell-to-cell transfer of glycosylphosphatidylinositol-anchored membrane proteins during sperm maturation. Mol Hum Reprod 2, 177-184. Kirchhoff, C., Pera, I., Derr, P., Yeung, C. H., and Cooper, T. (1997). The molecular biology of the sperm surface. Post-testicular membrane remodelling. Adv Exp Med Biol 424, 221-232.
Klebanoff, S. J., and Smith, D. C. (1970). The source of H2O2 for the uterine fluid-mediated sperm-inhibitory system. Biol Reprod 3, 236-242.
Koppers, A. J., De luliis, G. N., Finnie, J. M., McLaughlin, E. A., and Aitken, R. J. (2008). Significance of mitochondrial reactive oxygen species in the generation of oxidative stress in spermatozoa. J Clin Endocrinol Metab 93, 3199-3207.
Lapointe, S., and Sirard, M. A. (1998). Catalase and oviductal fluid reverse the decreased motility of bovine sperm in culture medium containing specific amino acids. J Androl 19, 31-36.
Lathrop, W. F., Carmichael, E. P., Myles, D. G., and Primakoff, P. (1990). cDNA cloning reveals the molecular structure of a sperm surface protein, PH-20, involved in sperm-egg adhesion and the wide distribution of its gene among mammals. J Cell Biol 111, 2939-2949.
Lightfoot, R. J., and Restall, B. J. (1971). Effects of site of insemination, sperm motility and genital tract contractions on transport of spermatozoa in the ewe. J Reprod Fertil 26, 1-13.
Lu, K. H., and Seidel, G. E., Jr. (2004). Effects of heparin and sperm concentration on cleavage and blastocyst development rates of bovine oocytes inseminated with flow cytometrically-sorted sperm. Theriogenology 62, 819-830.
Lutz, J. F., and Zarafshani, Z. (2008). Efficient construction of therapeutics, bioconjugates, biomaterials and bioactive surfaces using azide-alkyne “click” chemistry. Adv Drug Deliv Rev 60, 958-970.
Maccarrone, M. (2009). Endocannabinoids: friends and foes of reproduction. Prog Lipid Res 48, 344-354.
Maccarrone, M., Barboni, B., Paradisi, A., Bernabo, N., Gasperi, V., Pistilli, M. G., Fezza, F., Lucidi, P., and Mattioli, M. (2005). Characterization of the endocannabinoid system in boar spermatozoa and implications for sperm capacitation and acrosome reaction. J Cell Sci 118, 4393-4404.
Mahmoud, A. I., and Parrish, J. J. (1996). Oviduct fluid and heparin induce similar surface changes in bovine sperm during capacitation: a flow cytometric study using lectins. Mol Reprod Dev 43, 554-560.
Matthijs, A., Engel, B., and Woelders, H. (2003). Neutrophil recruitment and phagocytosis of boar spermatozoa after artificial insemination of sows, and the effects of inseminate volume, sperm dose and specific additives in the extender. Reproduction 125, 357-367.
McCauley, T. C., Zhang, H., Bellin, M. E., and Ax, R. L. (1999). Purification and characterization of fertility-associated antigen (FAA) in bovine seminal fluid. Mol Reprod Dev 54, 145-153.
Medeiros, C. M., and Parrish, J. J. (1996). Changes in lectin binding to bovine sperm during heparin-induced capacitation. Mol Reprod Dev 44, 525-532.
Mitchell, J. R., Senger, P. L., and Rosenberger, J. L. (1985). Distribution and retention of spermatozoa with acrosomal and nuclear abnormalities in the cow genital tract. J Anim Sci 61, 956-967.
Moura, A. A., Chapman, D. A., Koc, H., and Killian, G. J. (2006a). Proteins of the cauda epididymal fluid associated with fertility of mature dairy bulls. J Androl 27, 534-541.
Moura, A. A., Chapman, D. A., Koc, H., and Killian, G. J. (2007). A comprehensive proteomic analysis of the accessory sex gland fluid from mature Holstein bulls. Anim Reprod Sci 98, 169-188.
Moura, A. A., Koc, H., Chapman, D. A., and Killian, G. J. (2006b). Identification of proteins in the accessory sex gland fluid associated with fertility indexes of dairy bulls: a proteomic approach. J Androl 27, 201-211.
Mullins, K. J., and Saacke, R. G. (1989). Study of the functional anatomy of bovine cervical mucosa with special reference to mucus secretion and sperm transport. Anat Rec 225, 106-117.
Nagy, S., Jansen, J., Topper, E. K., and Gadella, B. M. (2003). A triple-stain flow cytometric method to assess plasma- and acrosome-membrane integrity of cryopreserved bovine sperm immediately after thawing in presence of egg-yolk particles. Biol Reprod 68, 1828-1835.
Novak, S., Ruiz-Sanchez, A., Dixon, W. T., Foxcroft, G. R., and Dyck, M. K. (2010a). Seminal plasma proteins as potential markers of relative fertility in boars. J Androl 31, 188-200.
Novak, S., Smith, T. A., Paradis, F., Burwash, L., Dyck, M. K., Foxcroft, G. R., and Dixon, W. T. (2010b). Biomarkers of in vivo fertility in sperm and seminal plasma of fertile stallions. Theriogenology 74, 956-967.
Oren-Benaroya, R., Kipnis, J., and Eisenbach, M. (2007). Phagocytosis of human post-capacitated spermatozoa by macrophages. Hum Reprod 22, 2947-2955.
Osheroff, J. E., Visconti, P. E., Valenzuela, J. P., Travis, A. J., Alvarez, J., and Kopf, G. S. (1999). Regulation of human sperm capacitation by a cholesterol efflux-stimulated signal transduction pathway leading to protein kinase A-mediated up-regulation of protein tyrosine phosphorylation. Mol Hum Reprod 5, 1017-1026.
Otero, M. C., and Nader-Macias, M. E. (2006). Inhibition of Staphylococcus aureus by H₂O₂-producing Lactobacillus gasseri isolated from the vaginal tract of cattle. Anim Reprod Sci 96, 35-46.
Parrish, J. J., and Foote, R. H. (1985). Fertility differences among male rabbits determined by heterospermic insemination of fluorochrome-labeled spermatozoa. Biol Reprod 33, 940-949.
Rathi, R., Colenbrander, B., Bevers, M. M., and Gadella, B. M. (2001). Evaluation of in vitro capacitation of stallion spermatozoa. Biol Reprod 65, 462-470.
Roberts, M. J., Bentley, M. D., and Harris, J. M. (2002). Chemistry for peptide and protein PEGylation. Adv Drug Deliv Rev 54, 459-476.
Rodriguez-Martinez, H., Kvist, U., Ernerudh, J., Sanz, L., and Calvete, J. J. (2011). Seminal plasma proteins: what role do they play? Am J Reprod Immunol 66 Suppl 1, 11-22.
Saeki, K., Nagao, Y., Hoshi, M., and Nagai, M. (1995). Effects of heparin, sperm concentration and bull variation on in vitro fertilization of bovine oocytes in a protein-free medium. Theriogenology 43, 751-759.
Salicioni, A. M., Platt, M. D., Wertheimer, E. V., Arcelay, E., Allaire, A., Sosnik, J., and Visconti, P. E. (2007). Signalling pathways involved in sperm capacitation. Soc Reprod Fertil Suppl 65, 245-259.
Schroter, S., Osterhoff, C., McArdle, W., and Iyell, R. (1999). The glycocalyx of the sperm surface. Hum Reprod Update 5, 302-313.
Scott, M. D., Murad, K. L., Koumpouras, F., Talbot, M., and Eaton, J. W. (1997). Chemical camouflage of antigenic determinants: stealth erythrocytes. Proc Natl Acad Sci USA 94, 7566-7571.
Senior, J., Delgado, C., Fisher, D., Tilcock, C., and Gregoriadis, G. (1991). Influence of surface hydrophilicity of liposomes on their interaction with plasma protein and clearance from the circulation: studies with poly(ethylene glycol)-coated vesicles. Biochim Biophys Acta 1062, 77-82.
Shannon, P., and Curson, B. (1972). Toxic effect and action of dead sperm on diluted bovine semen. J Dairy Sci 55, 614-620.
Shapiro, H. M., Natale, P. J., and Kamentsky, L. A. (1979). Estimation of membrane potentials of individual lymphocytes by flow cytometry. Proc Natl Acad Sci USA 76, 5728-5730.
Sharpe, J. C., and Evans, K. M. (2009). Advances in flow cytometry for sperm sexing. Theriogenology 71, 4-10.
Storey, B. T. (1997). Biochemistry of the induction and prevention of lipoperoxidative damage in human spermatozoa. Mol Hum Reprod 3, 203-213.
Storey, B. T. (2008). Mammalian sperm metabolism: oxygen and sugar, friend and foe. Int J Dev Biol 52, 427-437.
Sumner, J. B., and Dounce, A. L. (1937). Crystalline Catalase. Science 85, 366-367.
Tang, S., Garrett, C., and Baker, H. W. (1999). Comparison of human cervical mucus and artificial sperm penetration media. Hum Reprod 14, 2812-2817.
Teijeiro, J. M., Ignotz, G. G., and Marini, P. E. (2009). Annexin A2 is involved in pig (Sus scrofa)sperm-oviduct interaction. Mol Reprod Dev 76, 334-341.
Tejerina, F., Buranaamnuay, K., Saravia, F., Wallgren, M., and Rodriguez-Martinez, H. (2008). Assessment of motility of ejaculated, liquid-stored boar spermatozoa using computerized instruments. Theriogenology 69, 1129-1138.
Teramura, Y., and Iwata, H. (2009). Surface modification of islets with PEG-lipid for improvement of graft survival in intraportal transplantation. Transplantation 88, 624-630.
Tollner, T. L., Venners, S. A., Hollox, E. J., Yudin, A. I., Liu, X., Tang, G., Xing, H., Kays, R. J., Lau, T., Overstreet, J. W., et al. (2011). A Common Mutation in the Defensin DEFB126 Causes Impaired Sperm Function and Subfertility. Sci Transl Med 3, 92ra65.
Tollner, T. L., Yudin, A. I., Tarantal, A. F., Treece, C. A., Overstreet, J. W., and Cherr, G. N. (2008a). Beta-defensin 126 on the surface of macaque sperm mediates attachment of sperm to oviductal epithelia. Biol Reprod 78, 400-412.
Tollner, T. L., Yudin, A. I., Treece, C. A., Overstreet, J. W., and Cherr, G. N. (2008b). Macaque sperm coating protein DEFB126 facilitates sperm penetration of cervical mucus. Hum Reprod 23, 2523-2534.
Topfer-Petersen, E., Romero, A., Varela, P. F., Ekhlasi-Hundrieser, M., Dostalova, Z., Sanz, L., and Calvete, J. J. (1998). Spermadhesins: a new protein family. Facts, hypotheses and perspectives. Andrologia 30, 217-224.
Tosic, J., and Walton, A. (1950). Metabolism of spermatozoa. The formation and elimination of hydrogen peroxide by spermatozoa and effects on motility and survival. Biochem J 47, 199-212.
Vazquez, J. M., Martinez, E. A., Roca, J., Gil, M. A., Parrilla, I., Cuello, C., Carvajal, G., Lucas, X., and Vazquez, J. L. (2005). Improving the efficiency of sperm technologies in pigs: the value of deep intrauterine insemination. Theriogenology 63, 536-547.
Vazquez, J. M., Roca, J., Gil, M. A., Cuello, C., Parrilla, I., Vazquez, J. L., and Martinez, E. A. (2008). New developments in low-dose insemination technology. Theriogenology 70, 1216-1224.
Vernet, P., Fulton, N., Wallace, C., and Aitken, R. J. (2001). Analysis of reactive oxygen species generating systems in rat epididymal spermatozoa. Biol Reprod 65, 1102-1113.
Visconti, P. E., Ning, X., Fornes, M. W., Alvarez, J. G., Stein, P., Connors, S. A., and Kopf, G. S. (1999). Cholesterol efflux-mediated signal transduction in mammalian sperm: cholesterol release signals an increase in protein tyrosine phosphorylation during mouse sperm capacitation. Dev Biol 214, 429-443.
Waberski, D., Magnus, F., Mendonca Ferreira, F., Petrunkina, A. M., Weitze, K. F., and Topfer-Petersen, E. (2005). Importance of sperm-binding assays for fertility prognosis of porcine spermatozoa. Theriogenology 63, 470-484.
Williamson, P., Mattocks, K., and Schlegel, R. A. (1983). Merocyanine 540, a fluorescent probe sensitive to lipid packing. Biochim Biophys Acta 732, 387-393.
Wisniewski, J. R., Zougman, A., Nagaraj, N., and Mann, M. (2009). Universal sample preparation method for proteome analysis. Nat Methods 6, 359-362.
Woelders, H., and Matthijs, A. (2001). Phagocytosis of boar spermatozoa in vitro and in vivo. Reprod Suppl 58, 113-127.
Yeung, C. H. (2010). Aquaporins in spermatozoa and testicular germ cells: identification and potential role. Asian J Androl 12, 490-499.
Yudin, A. I., Tollner, T. L., Li, M. W., Treece, C. A., Overstreet, J. W., and Cherr, G. N. (2003). ESP13.2, a member of the beta-defensin family, is a macaque sperm surface-coating protein involved in the capacitation process. Biol Reprod 69, 1118-1128.
Yudin, A. I., Treece, C. A., Toliner, T. L., Overstreet, J. W., and Cherr, G. N. (2005). The carbohydrate structure of DEFB126, the major component of the cynomolgus Macaque sperm plasma membrane glycocalyx. J Membr Biol 207, 119-129.
Zalipsky, S., Brandeis, E., Newman, M. S., and Woodle, M. G. (1994). Long circulating, cationic liposomes containing amino-PEG-phosphatidylethanolamine. FEBS Lett 353, 71-74.

1. A conjugate comprising a membrane anchoring agent, polyethylene glycol and a functional molecule that is effective in increasing the lifespan of sperm in the female reproductive tract, wherein the functional molecule is attached to the polyethylene glycol by an attachment group.

2. The conjugate of claim 1, wherein the functional molecule is selected from the group consisting of: catalase; glutathione; CD55; CD59; CD73; DNaseI; SPAM1; polypeptides comprising an amino acid sequence selected from the group consisting of: SEQ ID NO: 1-163; and variants thereof.

3. (canceled)

4. The conjugate of claim 1, wherein the attachment group is selected from the group consisting of: ester amine reactive groups; maleimide; vinyl sulfone; iodoacetamide; orthopyridyl disulfide; hydrazide; benzotriazole; succinimidyl carbonate; and succinimidyl active esters based on priopionic butanoic acid.

5. The conjugate of claim 1, wherein the membrane anchoring agent is a lipid.

6. The conjugate of claim 5, wherein the lipid is selected from the group consisting of: cholesterol, diacylglycerolipids, dialkylglycerolipids, glycerophospholipids, sphingosine derived diacyl- and dialkyl-lipids, ceramide, phosphatidate, phosphatidyl choline, phosphatidyl ethanolamine, phosphatidyl serine, phosphatidyl inositol and phosphatidyl glycerol.

7. The conjugate of claim 1, wherein the conjugate comprises a structure of any one of FIGS. 1A-C.

8. A composition comprising a conjugate of claim 1 and a physiologically acceptable carrier.

9. A preparation comprising live sperm and a composition of claim 8.

10. (canceled)

11. A method for improving functionality and/or fertility of sperm, comprising contacting the sperm with an effective amount of a conjugate of claim 1.

12. The method of claim 11, wherein the functional molecule is selected from the group consisting of: catalase; glutathione; CD55; CD59; CD73; DNaseI; SPAM1; polypeptides comprising an amino acid sequence selected from the group consisting of: SEQ ID NO: 1-163; and variants thereof.

13. (canceled)

14. The method of claim 11, wherein the functional molecule is attached to the conjugate by means of an attachment group.

15. The method of claim 14, wherein the attachment group is selected from the group consisting of: ester amine reactive groups; maleimide; vinyl sulfone; iodoacetamide; orthopyridyl disulfide; hydrazide; benzotriazole; succinimidyl carbonate; and succinimidyl active esters based on priopionic and butanoic acids.

16. The method of claim 11, wherein the membrane anchoring agent is a lipid.

17. The method of claim 16, wherein the lipid is selected from the group consisting of: cholesterol, diacylglycerolipids, dialkylglycerolipids, glycerophospholipids, sphingosine derived diacyl- and dialkyl-lipids, ceramide, phosphatidate, phosphatidyl choline, phosphatidyl ethanolamine phosphatidyl serine, phosphatidyl inositol and phosphatidyl glycerol.

18. The method of claim 11, wherein the conjugate comprises a structure of any one of FIGS. 1A-C.

19. A method for preparing a composition for use in artificial insemination or in vitro fertilization, comprising:
(a) obtaining sperm from a mammal; and
(b) contacting the sperm with an effective amount of a composition of claim 8.

20-21. (canceled)

22. A method for cryopreserving sperm comprising:
(a) contacting the sperm with a cryoprotectant and a composition of claim 8; and
(b) storing the sperm at a temperature of about 4° C. to about −196° C.

23. (canceled)

24. A conjugate comprising a functional molecule of interest attached to a molecule of any one of FIGS. 1A-C, wherein the functional molecule of interest is selected from the group consisting of: proteins, carbohydrates and biotin.

25. The conjugate of claim 24, wherein the conjugate comprises a molecule of FIG. 1A and the attachment group (X) is selected from the group consisting of: ester amine reactive groups; maleimide; vinyl sulfone; iodoacetamide; orthopyridyl disulfide; hydrazide; benzotriazole; succinimidyl carbonate; and succinimidyl active esters based on priopionic and butanoic acids.

26. The conjugate of claim 24, wherein the further comprises a fluorescent group.

27. A composition comprising a conjugate of claim 24 and a physiologically acceptable carrier.

28. A method for attaching a functional molecule of interest to the surface of a cell, comprising contacting the cell with a conjugate of claim 24.

29. The method of claim 28, wherein the conjugate comprises the functional molecule of interest attached to a molecule of any one of FIGS. 1A-C.

30. The method of claim 28, wherein the attachment group is selected from the group consisting of: ester amine reactive groups; maleimide; vinyl sulfone; iodoacetamide; orthopyridyl disulfide; hydrazide; benzotriazole; succinimidyl carbonate; and succinimidyl active esters based on priopionic and beta acids.

31. The method of claim 28, wherein the conjugate further comprises a fluorescent group.
